(19) United States
(12) Patent Application Publication
(10) Pub. No.: US 2025/0237758 A1
(43) Pub. Date: Jul. 24, 2025

(54) CHILD SIZE ASSESSMENT IN CHILD SEAT

(71) Applicant: Volvo Car Corporation, Goteborg (SE)

(72) Inventors: Johan Rosenberg, Goteborg (SE); Sebastian Rutelin, Goteborg (SE)

(73) Assignee: Volvo Car Corporation, Goteborg (SE)

(21) Appl. No.: 18/417,328

(22) Filed: Jan. 19, 2024

(51) Int. Cl. G01S 13/86; A61B 5/00; A61B 5/05; A61B 5/107; G01S 13/89; G06V 20/40; G06V 20/59; G06V 40/20

(52) U.S. Cl. CPC G01S 13/867; A61B 5/0035; A61B 5/05; A61B 5/1079; A61B 5/6893; G01S 13/89; G06V 20/44; G06V 20/593; G06V 40/23; A61B 2503/06; A61B 2560/0443

(57) ABSTRACT

According to an embodiment, it is a system comprising a sensor module and a processor, wherein the processor is operable to: sense and acquire at least a portion of an interior of a vehicle via the sensor module, determine using the information of the interior of the vehicle via a machine learning module a first characteristic of a child safety seat positioned in the interior of the vehicle, retrieve using the first characteristic of the child safety seat positioned in the interior of the vehicle a specification information of the child safety seat, determine using the information of the interior of the vehicle via the machine learning module a second characteristic of a child positioned in the child safety seat, compare the second characteristic of the child with the specification information of the child safety seat, and recommend a suitability of the child safety seat.

### FIELD OF THE INVENTION

The present disclosure relates generally to the vehicle safety field. More specifically, the present disclosure pertains to techniques and approaches for child size assessment in a child safety seat.

### BACKGROUND

Today there is a big risk of children who are seated in vehicles and are not seated in child seats designed to their size and age range. A child seated in a child seat could be too big or small and the child seat will not give the full protection performance, or the child could be seated in the normal seats even if it is too small. By letting the vehicle or the system inform the parents on the potential issue, there is a greater chance that the child seated is better protected. There is a known problem with children and their seating in vehicles. This is normally targeted by information campaigns and legal requirements. Still, it is hard for parents to know and follow all recommendations.

Therefore, there is a need for a system and method for assessment of a child in a child seat in a vehicle and a need to inform the parents, driver, or the responsible party of the potential issue regarding non-suitability, incorrect installation, etc., so that there is a greater chance that the child seated in the child seat is better protected.

### SUMMARY

The following presents a summary to provide a basic understanding of one or more embodiments described herein. This summary is not intended to identify key or critical elements or delineate any scope of the different embodiments and/or any scope of the claims. The sole purpose of the summary is to present some concepts in a simplified form as a prelude to the more detailed description presented herein.

According to an embodiment, it is a system comprising a sensor module comprising a radar sensor and an image sensor; a processor; and a memory communicably coupled to the processor comprising computer-readable instructions that when executed by the processor cause the to: sense, at least a portion of an interior of a vehicle, via the sensor module; acquire, information of the interior of the vehicle, via the sensor module; determine, using the information of the interior of the vehicle via a machine learning module, a first characteristic of a child safety seat positioned in the interior of the vehicle; retrieve, using the first characteristic of the child safety seat positioned in the interior of the vehicle, a specification information of the child safety seat; determine, using the information of the interior of the vehicle via the machine learning module, a second characteristic of a child positioned in the child safety seat; compare, the second characteristic of the child with the specification information of the child safety seat; and recommend, a suitability of the child safety seat.

According to an embodiment, it is a method comprising sensing, at least a portion of an interior of a vehicle, via a sensor module, wherein the sensor module comprises a radar sensor and an image sensor; acquiring information of the interior of the vehicle, via the sensor module; determining, using the information of the interior of the vehicle via a machine learning module, a first characteristic of a child safety seat positioned in the interior of the vehicle; retrieving, using the first characteristic of the child safety seat positioned in the interior of the vehicle, a specification information of the child safety seat; determining, using the information of the interior of the vehicle via the machine learning module, a second characteristic of a child positioned in the child safety seat; comparing the second characteristic of the child with the specification information of the child safety seat; and recommending a suitability of the child safety seat.

According to an embodiment, it is a non-transitory computer-readable medium having stored thereon instructions executable by a computer system to perform operations comprising, sensing, at least a portion of an interior of a vehicle, via a sensor module, wherein the sensor module comprises a radar sensor and an image sensor; acquiring, information of the interior of the vehicle, via the sensor module; determining, using the information of the interior of the vehicle via a machine learning module, a first characteristic of a child safety seat positioned in the interior of the vehicle; retrieving, using the first characteristic of the child safety seat positioned in the interior of the vehicle, a specification information of the child safety seat; determining, using the information of the interior of the vehicle via the machine learning module, a second characteristic of a child positioned in the child safety seat; comparing, the second characteristic of the child with the specification information of the child safety seat; and recommending, a suitability of the child safety seat.

### BRIEF DESCRIPTION OF THE FIGURES

Aspects of the present invention will now be described in more detail, with reference to the appended drawings showing exemplary embodiments of the present invention, in which:

FIG. 1 shows a child safety seat detection and installation guidance system according to an embodiment.

FIG. 2A shows an example of Child Safety Seat Identification according to an embodiment.

FIG. 2B shows an example for accessing Installation Requirements and Usage Limits according to an embodiment.

FIG. 2C shows a block diagram of a system executing a method for child safety seat detection and installation guidance according to an embodiment.

FIG. 2D shows an implementation of a connector for child safety seat detection according to an embodiment.

FIG. 2E shows a diagram of an embodiment showing a child safety seat, having an electronic unit, including a memory loaded with child safety seat data, and a child seat interface for communication with the vehicle.

FIG. 2F shows a guidance system for placing and harnessing a child according to an embodiment.

FIG. 2G shows a guidance system for adjusting a part of child safety seat according to a child according to an embodiment.

FIG. 2H shows a block diagram for the method depicting the steps involved in child size assessment in a child seat according to an embodiment.

FIG. 2I shows a block diagram for the system for child size assessment in a child seat according to an embodiment.

FIG. 2J shows a block diagram for the non-transitory computer medium for storing the steps involved in child size assessment in a child seat according to an embodiment.

FIG. 2K shows a block diagram showing the steps involved in child seat suitability assessment according to an embodiment.

FIG. 3 shows a child safety seat/child restraint system with various sensors according to an embodiment.

FIG. 4 shows a block diagram for a method depicting the steps involved in a computer vision system for detecting child safety seat installation.

FIG. 5 shows a checklist for child safety seat installation according to an embodiment.

FIG. 6 shows a block diagram of electronic components of a vehicle according to an embodiment.

FIG. 7 shows a sample representation of the status of installation on the infotainment system according to an embodiment.

FIG. 8 shows an alert about a child safety seat on a display when the status of the seat installation is changed at any time during a ride in the vehicle.

FIG. 9A shows a structure of the neural network/machine learning model with a feedback loop according to an embodiment.

FIG. 9B shows a structure of the neural network/machine learning model with reinforcement learning according to an embodiment.

FIG. 9C shows an example block diagram for detecting an improper installation of a child safety seat and/or placing and harnessing the child in the child safety seat using a machine learning model.

FIG. 10A shows an example flow chart for detecting an improper installation of a child safety seat and/or placing and harnessing the child in the child safety seat using a machine learning model.

FIG. 10B shows example steps for determining suitability of a child safety seat based on child's size, weight, age, fit, and behavior according to an embodiment.

FIG. 11 shows a block diagram of the cyber security module in view of the system and server according to an embodiment.

### DETAILED DESCRIPTION

For simplicity and clarity of illustration, the figures illustrate the general manner of construction. The description and figures may omit the descriptions and details of well-known features and techniques to avoid unnecessarily obscuring the present disclosure. The figures exaggerate the dimensions of some of the elements relative to other elements to help improve understanding of embodiments of the present disclosure. The same reference numeral in different figures denotes the same element.

Although the detailed description herein contains many specifics for the purpose of illustration, a person of ordinary skill in the art will appreciate that many variations and alterations to the details are considered to be included herein.

Accordingly, the embodiments herein are without any loss of generality to, and without imposing limitations upon, any claims set forth. The terminology used herein is for the purpose of describing particular embodiments only and is not limiting. Unless defined otherwise, all technical and scientific terms used herein have the same meaning as commonly understood by one with ordinary skill in the art to which this disclosure belongs.

As used herein, the articles “a” and “an” used herein refer to one or to more than one (i.e., to at least one) of the grammatical object of the article. By way of example, “an element” means one element or more than one element. Moreover, usage of articles “a” and “an” in the subject specification and annexed drawings construe to mean “one or more” unless specified otherwise or clear from context to mean a singular form.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

As used herein, the terms “first,” “second,” “third,” and the like in the description and in the claims, if any, distinguish between similar elements and do not necessarily describe a particular sequence or chronological order. The terms are interchangeable under appropriate circumstances such that the embodiments herein are, for example, capable of operation in sequences other than those illustrated or otherwise described herein. Furthermore, the terms “include,” “have,” and any variations thereof, cover a non-exclusive inclusion such that a process, method, system, article, device, or apparatus that comprises a list of elements is not necessarily limiting to those elements, but may include other elements not expressly listed or inherent to such process, method, system, article, device, or apparatus.

As used herein, the terms “left,” “right,” “front,” “back,” “top,” “bottom,” “over,” “under” and the like in the description and in the claims, if any, are for descriptive purposes and not necessarily for describing permanent relative positions. The terms so used are interchangeable under appropriate circumstances such that the embodiments of the apparatus, methods, and/or articles of manufacture described herein are, for example, capable of operation in other orientations than those illustrated or otherwise described herein.

No element act, or instruction used herein is critical or essential unless explicitly described as such. Furthermore, the term “set” includes items (e.g., related items, unrelated items, a combination of related items and unrelated items, etc.) and may be interchangeable with “one or more”. Where only one item is intended, the term “one” or similar language is used. Also, the terms “has,” “have,” “having,” or the like are open-ended terms. Further, the phrase “based on” means “based, at least in part, on” unless explicitly stated otherwise.

As used herein, the terms “system,” “device,” “unit,” and/or “module” refer to a different component, component portion, or component of the various levels of the order. However, other expressions that achieve the same purpose may replace the terms.

As used herein, the terms “couple,” “coupled,” “couples,” “coupling,” and the like refer to connecting two or more elements mechanically, electrically, and/or otherwise. Two or more electrical elements may be electrically coupled together, but not mechanically or otherwise coupled together. Coupling may be for any length of time, e.g., permanent, or semi-permanent or only for an instant. “Electrical coupling” includes electrical coupling of all types. The absence of the word “removably,” “removable,” and the like, near the word “coupled”, and the like, does not mean that the coupling, etc., in question is or is not removable.

As used herein, the term “or” means an inclusive “or” rather than an exclusive “or.” That is, unless specified otherwise, or clear from context, “X employs A or B” means any of the natural inclusive permutations. That is, if X employs A; X employs B; or X employs both A and B, then “X employs A or B” is satisfied under any of the foregoing instances.

As used herein, the term “real-time” refers to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Other specific forms may embody the present invention without departing from its spirit or characteristics. The described embodiments are in all respects illustrative and not restrictive. Therefore, the appended claims rather than the description herein indicate the scope of the invention. All variations which come within the meaning and range of equivalency of the claims are within their scope.

As used herein, the term “component” broadly construes hardware, firmware, and/or a combination of hardware, firmware, and software.

Digital electronic circuitry, or in computer software, firmware, or hardware, including the structures disclosed in this specification and their structural equivalents, or in combinations of one or more of them may realize the implementations and all of the functional operations described in this specification. Implementations may be as one or more computer program products, i.e., one or more modules of computer program instructions encoded on a computer-readable medium for execution by, or to control the operation of, data processing apparatus. The computer-readable medium may be a machine-readable storage device, a machine-readable storage substrate, a memory device, a composition of matter affecting a machine-readable propagated signal, or a combination of one or more of them.

The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that encodes information for transmission to a suitable receiver apparatus.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a standalone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as, special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of a digital computer. A processor will receive instructions and data from a read-only memory or a random-access memory or both. Elements of a computer can include a processor for performing instructions and one or more memory devices for storing instructions and data. A computer will also include, or is operatively coupled to receive data, transfer data or both, to/from one or more mass storage devices for storing data e.g., magnetic disks, magneto optical disks, optical disks, or solid-state disks. However, a computer need not have such devices. Moreover, another device, e.g., a mobile telephone, a personal digital assistant (PDA), a mobile audio player, a Global Positioning System (GPS) receiver, etc. may embed a computer. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disc Read-Only Memory (CD ROM) disks, Digital Versatile Disk-Read-Only Memory (DVD-ROM) disks) and solid-state disks. Special purpose logic circuitry may supplement or incorporate the processor and the memory.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

A computing system that includes a back-end component, e.g., a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components, may realize implementations described herein. Any appropriate form or medium of digital data communication, e.g., a communication network may interconnect the components of the system. Examples of communication networks include a Local Area Network (LAN) and a Wide Area Network (WAN), e.g., Intranet and Internet.

The computing system may include clients and servers. A client and server are remote from each other and typically interact through a communication network. The relationship of the client and server arises by virtue of computer programs running on the respective computers and having a client-server relationship with each other.

Embodiments of the present invention may comprise or utilize a special purpose or general purpose computer including computer hardware. Embodiments within the scope of the present invention may also include physical and other computer-readable media for carrying or storing computer-executable instructions and/or data structures. Such computer-readable media can be any media accessible by a general purpose or special purpose computer system. Computer-readable media that store computer-executable instructions are physical storage media. Computer-readable media that carry computer-executable instructions are transmission media. Thus, by way of example and not limitation, embodiments of the invention can comprise at least two distinct kinds of computer-readable media: physical computer-readable storage media and transmission computer-readable media.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in a non-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specification and drawings are illustrative rather than restrictive. Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Module (NIC), and then eventually transferred to computer system RAM and/or to less volatile computer-readable physical storage media at a computer system. Thus, computer system components that also (or even primarily) utilize transmission media may include computer-readable physical storage media.

Computer-executable instructions comprise, for example, instructions and data which cause a general purpose computer, special purpose computer, or special purpose processing device to perform a certain function or group of functions. The computer-executable instructions may be, for example, binary, intermediate format instructions such as assembly language, or even source code. Although the subject matter herein described is in a language specific to structural features and/or methodological acts, the described features or acts described do not limit the subject matter defined in the claims. Rather, the herein described features and acts are example forms of implementing the claims.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the invention may be practiced in network computing environments with many types of computer system configurations including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the invention. In a distributed system environment, program modules may be located in both local and remote memory storage devices.

As used herein, the term “cryptographic protocol” is also known as security protocol or encryption protocol. It is an abstract or concrete protocol that performs a security-related function and applies cryptographic methods often as sequences of cryptographic primitives.

As used herein, the term “IoT” stands for Internet of Things which describes the network of physical objects “things” or objects embedded with sensors, software, and other technologies for the purpose of connecting and exchanging data with other devices and systems over the internet.

As used herein “machine learning” refers to algorithms that give a computer the ability to learn without explicit programming, including algorithms that learn from and make predictions about data. Machine learning techniques include, but are not limited to, support vector machine, artificial neural network (ANN) (also referred to herein as a “neural net”), deep learning neural network, logistic regression, discriminant analysis, random forest, linear regression, rules-based machine learning, Naive Bayes, nearest neighbor, decision tree, decision tree learning, and hidden Markov, etc. For the purposes of clarity, part of a machine learning process can use algorithms such as linear regression or logistic regression. However, using linear regression or another algorithm as part of a machine learning process is distinct from performing a statistical analysis such as regression with a spreadsheet program. The machine learning process can continually learn and adjust the classifier as new data becomes available and does not rely on explicit or rules-based programming. The ANN may be featured with a feedback loop to adjust the system output dynamically as it learns from the new data as it becomes available. In machine learning, backpropagation and feedback loops are used to train the Artificial Intelligence/Machine Learning (AI/ML) model improving the model's accuracy and performance over time.

As used herein, the term “data mining” is a process used to turn raw data into useful information.

As used herein, the term “data acquisition” is the process of sampling signals that measure real world physical conditions and converting the resulting samples into digital numeric values that a computer manipulates. Data acquisition systems typically convert analog waveforms into digital values for processing. The components of data acquisition systems include sensors to convert physical parameters to electrical signals, signal conditioning circuitry to convert sensor signals into a form that can be converted to digital values, and analog-to-digital converters to convert conditioned sensor signals to digital values. Stand-alone data acquisition systems are often called data loggers.

As used herein, the term “dashboard” is a type of interface that visualizes particular Key Performance Indicators (KPIs) for a specific goal or process. It is based on data visualization and infographics.

As used herein, a “database” is a collection of organized information so that it can be easily accessed, managed, and updated. Computer databases typically contain aggregations of data records or files.

As used herein, the term “data set” (or “dataset”) is a collection of data. In the case of tabular data, a data set corresponds to one or more database tables, where every column of a table represents a particular variable, and each row corresponds to a given record of the data set in question.

As used herein, a “sensor” is a device that measures physical input from its environment and converts it into data that is interpretable by either a human or a machine. Most sensors are electronic, which presents electronic data, but some are simpler, such as a glass thermometer, which presents visual data.

The term “infotainment system” or “in-vehicle infotainment system” (IVI) as used herein refers to a combination of vehicle systems which are used to deliver entertainment and information. In an example, the information may be delivered to the driver and the passengers of a vehicle/occupants through audio/video interfaces, control elements like touch screen displays, button panel, voice commands, and more. Some of the main components of an in-vehicle infotainment systems are integrated head-unit, heads-up display, high-end Digital Signal Processors (DSPs), and Graphics Processing Units (GPUs) to support multiple displays, operating systems, Controller Area Network (CAN), Low-Voltage Differential Signaling (LVDS), and other network protocol support (as per the requirement), connectivity modules, automotive sensors integration, digital instrument cluster, etc.

The term “environment” or “surrounding” as used herein refers to surroundings and the space in which a vehicle is navigating. It refers to dynamic surroundings in which a vehicle is navigating which includes other vehicles, obstacles, pedestrians, lane boundaries, traffic signs and signals, speed limits, potholes, snow, water logging etc.

The term “autonomous mode” as used herein refers to an operating mode which is independent and unsupervised.

The term “vehicle” as used herein refers to a thing used for transporting people or goods. Automobiles, cars, trucks, buses etc., are examples of vehicles.

The term “autonomous vehicle” also referred to as self-driving vehicle, driverless vehicle, robotic vehicle as used herein refers to a vehicle incorporating vehicular automation, that is, a ground vehicle that can sense its environment and move safely with little or no human input. Self-driving vehicles combine a variety of sensors to perceive their surroundings, such as thermographic cameras, Radio Detection and Ranging (RADAR), Light Detection and Ranging (LIDAR), Sound Navigation and Ranging (SONAR), Global Positioning System (GPS), odometry and inertial measurement unit. Control systems, designed for the purpose, interpret sensor information to identify appropriate navigation paths, as well as obstacles and relevant signage.

The term “communication module” or “communication system” as used herein refers to a system which enables the information exchange between two points. The process of transmission and reception of information is called communication. The elements of communication include but are not limited to a transmitter of information, channel or medium of communication and a receiver of information.

The term “autonomous communication” as used herein comprises communication over a period with minimal supervision under different scenarios and is not solely or completely based on pre-coded scenarios or pre-coded rules or a predefined protocol. Autonomous communication, in general, happens in an independent and an unsupervised manner.

The term “connection” as used herein refers to a communication link. It refers to a communication channel that connects two or more devices for the purpose of data transmission. It may refer to a physical transmission medium such as a wire, or to a logical connection over a multiplexed medium such as a radio channel in telecommunications and computer networks. A channel is used for the information transfer of, for example, a digital bit stream, from one or several senders to one or several receivers. A channel has a certain capacity for transmitting information, often measured by its bandwidth in Hertz (Hz) or its data rate in bits per second. For example, a Vehicle-to-Vehicle (V2V) communication may wirelessly exchange information about the speed, location and heading of surrounding vehicles.

The term “communication” as used herein refers to the transmission of information and/or data from one point to another. Communication may be by means of electromagnetic waves. Communication is also a flow of information from one point, known as the source, to another, the receiver. Communication comprises one of the following: transmitting data, instructions, information or a combination of data, instructions, and information.

Further, the communication apparatus is configured on a computer with the communication function and is connected for bidirectional communication with the on-vehicle emergency report apparatus by a communication line through a radio station and a communication network such as a public telephone network or by satellite communication through a communication satellite.

The term “Vehicle-to-Vehicle (V2V) communication” refers to the technology that allows vehicles to broadcast and receive messages. The messages may be omni-directional messages, creating a 360-degree “awareness” of other vehicles in proximity. Vehicles may be equipped with appropriate software (or safety applications) that can use the messages from surrounding vehicles to determine potential crash threats as they develop.

The term “V2X communication” as used herein refers to transmission of information from a vehicle to any entity that may affect the vehicle, and vice versa. Depending on the underlying technology employed, there are two types of V2X communication technologies: cellular networks and other technologies that support direct device-to-device communication (such as Dedicated Short-Range Communication (DSRC), Port Community System (PCS), Bluetooth®, Wi-Fi®, etc.).

The term “protocol” as used herein refers to a procedure required to initiate and maintain communication; a formal set of conventions governing the format and relative timing of message exchange between two communications terminals; a set of conventions that govern the interactions of processes, devices, and other components within a system; a set of signaling rules used to convey information or commands between boards connected to the bus; a set of signaling rules used to convey information between agents; a set of semantic and syntactic rules that determine the behavior of entities that interact; a set of rules and formats (semantic and syntactic) that determines the communication behavior of simulation applications; a set of conventions or rules that govern the interactions of processes or applications between communications terminals; a formal set of conventions governing the format and relative timing of message exchange between communications terminals; a set of semantic and syntactic rules that determine the behavior of functional units in achieving meaningful communication; a set of semantic and syntactic rules for exchanging information.

The term “communication protocol” as used herein refers to standardized communication between any two systems. An example communication protocol is a DSRC protocol. The DSRC protocol uses a specific frequency band (e.g., 5.9 GHZ) and specific message formats (such as the Basic Safety Message, Signal Phase and Timing, and Roadside Alert) to enable communications between vehicles and infrastructure components, such as traffic signals and roadside sensors. DSRC is a standardized protocol, and its specifications are maintained by various organizations, including the IEEE and SAE International.

The term “bidirectional communication” as used herein refers to an exchange of data between two components. In an example, the first component can be a vehicle and the second component can be an infrastructure that is enabled by a system of hardware, software, and firmware.

The term “alert” or “alert signal” refers to a communication to attract attention. An alert may include visual, tactile, audible alert, and a combination of these alerts to warn drivers or occupants. These alerts allow receivers, such as drivers or occupants, the ability to react and respond quickly.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer access transmission media that can include a network and/or data links which carry desired program code in the form of computer-executable instructions or data structures. The scope of computer-readable media includes combinations of the above, that enable the transport of electronic data between computer systems and/or modules and/or other electronic devices. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes. The computers use common communication protocols over digital interconnections to communicate with each other. For example, subsystems may comprise the cloud. Cloud refers to servers that are accessed over the Internet, and the software and databases that run on those servers.

The term “electronic control unit” (ECU), also known as an “electronic control module” (ECM), is usually a module that controls one or more subsystems. Herein, an ECU may be installed in a car or other motor vehicle. It may refer to many ECUs, and can include but not limited to, Engine Control Module (ECM), Powertrain Control Module (PCM), Transmission Control Module (TCM), Brake Control Module (BCM) or Electronic Brake Control Module (EBCM), Central Control Module (CCM), Central Timing Module (CTM), General Electronic Module (GEM), Body Control Module (BCM), and Suspension Control Module (SCM). ECUs together are sometimes referred to collectively as the vehicles' computer or vehicles' central computer and may include separate computers. In an example, the electronic control unit can be an embedded system in automotive electronics. In another example, the electronic control unit is wirelessly coupled with automotive electronics.

The terms “non-transitory computer-readable medium” and “computer-readable medium” include a single medium or multiple media such as a centralized or distributed database, and/or associated caches and servers that store one or more sets of instructions. Further, the terms “non-transitory computer-readable medium” and “computer-readable medium” include any tangible medium that is capable of storing, encoding, or carrying a set of instructions for execution by a processor that, for example, when executed, cause a system to perform any one or more of the methods or operations disclosed herein. As used herein, the term “computer readable medium” is expressly defined to include any type of computer readable storage device and/or storage disk and to exclude propagating signals.

The term “Vehicle Data bus” as used herein represents the interface to the vehicle data bus (e.g., CAN, LIN, Ethernet/IP, FlexRay, and MOST) that may enable communication between the Vehicle on-board equipment (OBE) and other vehicle systems to support connected vehicle applications.

The term, “handshaking” refers to an exchange of predetermined signals between agents connected by a communications channel to assure each that it is connected to the other (and not to an imposter). This may also include the use of passwords and codes by an operator. Handshaking signals are transmitted back and forth over a communications network to establish a valid connection between two stations. A hardware handshake uses dedicated wires such as the request-to-send (RTS) and clear-to-send (CTS) lines in an Recommended Standard 232 (RS-232) serial transmission. A software handshake sends codes such as “synchronize” (SYN) and “acknowledge” (ACK) in a Transmission Control Protocol/Internet Protocol (TCP/IP) transmission.

The term “child safety seat” refers to a specialized seating device designed to protect infants and children in vehicles. Alternate terms for a child safety seat include, Child restraint system, Infant car seat, Toddler car seat, Baby seat, Car safety seat, Car seat for children, Child car seat, Booster seat (a type of child safety seat used for older children to elevate them for proper seat belt fit), Child harness, Infant carrier. These terms are often used interchangeably, and their usage may vary depending on the region and specific regulations governing child passenger safety. Child safety seats come in various types tailored to children's ages and sizes. These types include rear-facing infant seats, convertible seats, forward-facing seats, booster seats (high-back and backless), all-in-one seats, convertible booster seats, and youth booster seats. An “all-in-one” child safety seat, also known as a “3-in-1” or “4-in-1” car seat, is a versatile and convertible car seat designed to accommodate children as they grow and progress through various stages of development, from infancy to toddlerhood and beyond.

As used herein, the term “driver” refers to such an occupant, even when that occupant is not actually driving the vehicle but is situated in the vehicle so as to be able to take over control and function as the driver of the vehicle when the vehicle control system hands over control to the occupant or driver or when the vehicle control system is not operating in an autonomous or semi-autonomous mode.

As used herein, the term “step-by-step instructions” refers to a set of clear and organized guidelines that break down a process or task into a series of sequential steps. These instructions provide a systematic approach to help users or individuals understand and perform a specific action or complete a task successfully.

As used herein, the term “digital model” refers to a computerized representation or simulation of an object, system, process, or concept. It is created using digital data and algorithms to replicate the characteristics and behavior of the real-world entity in a virtual environment. For example, digital models may be a 3D model representing objects or spaces in a three-dimensional format. They capture the shape, structure, and appearance of the physical entity and can be viewed and manipulated from different angles.

As used herein, the term “information system” refers to a set of interconnected components, including hardware, software, databases, networks, and people that work together to collect, store, process, transmit, and retrieve information in an organized and meaningful way. It involves the use of technology, processes, and resources to manage data and support decision-making, coordination, control, feedback, analysis, and other activities. For example, an infotainment system can be considered a type of information system. An infotainment system combines information and entertainment features within a vehicle or other consumer electronic devices. It typically includes components such as a display screen, audio system, navigation system, and connectivity features.

As used herein, the term “tautness” in relation to a seat belt refers to the state of the seat belt being properly and tightly secured around the occupant. When a seat belt is properly fastened, it should be taut or snug against the occupant's body, without any excessive slack or looseness. Ensuring the tautness of a seat belt is crucial for its effectiveness in providing protection during a vehicle collision or sudden stop. A properly tightened seat belt helps to restrain the occupant and prevent them from being thrown forward or out of their seat in the event of a crash. It is also referred to as tightness of the seat belt.

As used herein, the term “seat belt payout” refers to the process of the seat belt webbing being released from the retractor mechanism, allowing the occupant to pull the seat belt out and put it on. The payout of a seat belt is important because it determines how much slack or length of the belt is allowed to be released for the occupant to use. The payout mechanism is typically designed to be smooth and controlled, allowing the occupant to easily pull the belt out to the desired length and then locking in place when the occupant stops pulling or when the belt is subjected to sudden deceleration, such as in a crash.

As used herein, the term “attachment point” also known as child restraint anchor points or Lower Anchors and Tethers for Children (LATCH) system, refers to designated locations in a vehicle where the child safety seat can be securely installed.

As used herein, the term “routing” refers to a process of correctly threading the vehicle's seat belt through the designated belt path of the child safety seat. Routing is guiding the seat belt through specific channels or guides on the child seat to ensure it is positioned correctly and tightly secured.

As used herein, the term “indicator” refers to a device, instrument, or sign that provides information, signals, or evidence about a specific condition, state, or process. It is used to convey or display information visually, audibly, or through other means to indicate the presence, absence, level, quality, or status of something (example, installation accuracy).

The term “computer vision module” or “computer vision system” allows the vehicle to “see” and interpret the world around it. This system uses a combination of cameras, sensors, and other technologies such as Radio Detection and Ranging (RADAR), Light Detection and Ranging (LIDAR), Sound Navigation and Ranging (SONAR), Global Positioning System (GPS), and Machine learning algorithms, etc., to collect visual data about the vehicle's surroundings and to analyze that data in real-time. The computer vision system is designed to perform a range of tasks, including object detection, lane detection, and pedestrian recognition. It uses deep learning algorithms and other machine learning techniques to analyze the visual data and make decisions about how to control the vehicle.

The term “cyber security” as used herein refers to application of technologies, processes, and controls to protect systems, networks, programs, devices, and data from cyber-attacks.

The term “cyber security module” as used herein refers to a module comprising application of technologies, processes, and controls to protect systems, networks, programs, devices and data from cyber-attacks and threats. It aims to reduce the risk of cyber-attacks and protect against the unauthorized exploitation of systems, networks, and technologies. It includes, but is not limited to, critical infrastructure security, application security, network security, cloud security, Internet of Things (IoT) security.

The term “encrypt” used herein refers to securing digital data using one or more mathematical techniques, along with a password or “key” used to decrypt the information. It refers to converting information or data into a code, especially to prevent unauthorized access. It may also refer to concealing information or data by converting it into a code. It may also be referred to as cipher, code, encipher, encode.

The term “decrypt” used herein refers to the process of converting an encrypted message back to its original format. It is generally a reverse process of encryption. It decodes the encrypted information so that only an authorized user can decrypt the data because decryption requires a secret key or password. This term could be used to describe a method of unencrypting the data manually or unencrypting the data using the proper codes or keys.

The term “cyber security threat” used herein refers to any possible malicious attack that seeks to unlawfully access data, disrupt digital operations, or damage information. A malicious act includes but is not limited to damaging data, stealing data, or disrupting digital life in general. Cyber threats include, but are not limited to, malware, spyware, phishing attacks, ransomware, zero-day exploits, trojans, advanced persistent threats, wiper attacks, data manipulation, data destruction, rogue software, malvertising, unpatched software, computer viruses, man-in-the-middle attacks, data breaches, Denial of Service (DOS) attacks, and other attack vectors.

The term “hash value” used herein can be thought of as fingerprints for files. The contents of a file are processed through a cryptographic algorithm, and a unique numerical value, the hash value, is produced that identifies the contents of the file. If the contents are modified in any way, the value of the hash will also change significantly. Example algorithms used to produce hash values: the Message Digest-5 (MD5) algorithm and Secure Hash Algorithm-1 (SHA1).

The term “integrity check” as used herein refers to the checking for accuracy and consistency of system related files, data, etc. It may be performed using checking tools that can detect whether any critical system files have been changed, thus enabling the system administrator to look for unauthorized alteration of the system. For example, data integrity corresponds to the quality of data in the databases and to the level by which users examine data quality, integrity, and reliability. Data integrity checks verify that the data in the database is accurate, and functions as expected within a given application.

The term “alarm” as used herein refers to a trigger when a component in a system or the system fails or does not perform as expected. The system may enter an alarm state when a certain event occurs. An alarm indication signal is a visual signal to indicate the alarm state. For example, when a cyber security threat is detected, a system administrator may be alerted via sound alarm, a message, a glowing LED, a pop-up window, etc. Alarm indication signal may be reported downstream from a detecting device, to prevent adverse situations or cascading effects.

The term “stature” refers to a person's height, especially when discussing their physical build or appearance. It is a term commonly used to describe how tall or short a person is in relation to others.

The term “behavior” as used herein refers to the behavior of a child in a child seat. It encompasses how a child behaves or acts while seated in a specially designed safety seat or restraint system for children. This behavior can vary widely depending on the child's age, temperament, comfort level, and the circumstances surrounding the use of the child seat. Desirable behaviors in a child seat typically include but are not limited to sitting still, being properly secured, and buckled in, and not engaging in activities that could compromise safety, such as unbuckling the seat belt or harness. Undesirable behaviors may include restlessness, crying, or attempting to escape from the seat. Behavior analysis analyzes images and video of the child in the child safety seat in real-time and the history of the child in the child safety seat and determines desirable and undesirable behaviors of the child for current ride and further used in the decision making on suitability of the child safety seat.

The term “suitability” as used herein in relation to a child safety seat refers to how well a particular child safety seat or restraint system matches the specific needs, size, age, and weight of a child. It assesses whether the seat is appropriate for the child in question in terms of providing effective protection and safety during transportation.

Aspects of the one or more embodiments described herein are described with reference to flowchart illustrations and/or block diagrams of methods, apparatus (systems), and computer program products according to one or more embodiments described herein. Each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer and/or other programmable data processing apparatus to produce a machine, such that the instructions, which execute via the processor of the computer or other programmable data processing apparatus, can create means for implementing the functions/acts specified in the flowchart and/or block diagram block or blocks. These computer readable program instructions can also be stored in a computer readable storage medium that can direct a computer, a programmable data processing apparatus and/or other devices to function in a particular manner, such that the computer readable storage medium having instructions stored therein can comprise an article of manufacture including instructions which can implement aspects of the function/act specified in the flowchart and/or block diagram block or blocks. The computer readable program instructions can also be loaded onto a computer, other programmable data processing apparatus and/or other device to cause a series of operational acts to be performed on the computer, other programmable apparatus and/or other device to produce a computer implemented process, such that the instructions which execute on the computer, other programmable apparatus and/or other device implement the functions/acts specified in the flowchart and/or block diagram block or blocks.

The flowcharts and block diagrams in the figures illustrate the architecture, functionality and/or operation of possible implementations of systems, computer-implementable methods and/or computer program products according to one or more embodiments described herein. In this regard, each block in the flowchart or block diagrams can represent a module, segment and/or portion of instructions, which comprises one or more executable instructions for implementing the specified logical function(s). In one or more alternative implementations, the functions noted in the blocks can occur out of the order noted in the Figures. For example, two blocks shown in succession can be executed substantially concurrently, and/or the blocks can sometimes be executed in the reverse order, depending upon the functionality involved. It will also be noted that each block of the block diagrams and/or flowchart illustration, and/or combinations of blocks in the block diagrams and/or flowchart illustration, can be implemented by special purpose hardware-based systems that can perform the specified functions and/or acts and/or carry out one or more combinations of special purpose hardware and/or computer instructions.

While the subject matter described herein is in the general context of computer-executable instructions of a computer program product that runs on a computer and/or computers, those skilled in the art will recognize that the one or more embodiments herein also can be implemented in combination with one or more other program modules. Program modules include routines, programs, components, data structures, and/or the like that perform particular tasks and/or implement particular abstract data types. Moreover, other computer system configurations, including single-processor and/or multiprocessor computer systems, mini-computing devices, mainframe computers, as well as computers, hand-held computing devices (e.g., PDA, phone), microprocessor-based or programmable consumer and/or industrial electronics and/or the like can practice the herein described computer-implemented methods. Distributed computing environments, in which remote processing devices linked through a communications network perform tasks, can also practice the illustrated aspects. However, stand-alone computers can practice one or more, if not all aspects of the one or more embodiments described herein. In a distributed computing environment, program modules can be located in both local and remote memory storage devices.

As used in this application, the terms “component,” “system,” “platform,” “interface,” and/or the like, can refer to and/or can include a computer-related entity or an entity related to an operational machine with one or more specific functionalities. The entities described herein can be either hardware, a combination of hardware and software, software, or software in execution. For example, a component can be, but is not limited to being, a process running on a processor, a processor, an object, an executable, a thread of execution, a program and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system and/or across a network such as the Internet with other systems via the signal). As another example, a component can be an apparatus with specific functionality provided by mechanical parts operated by electric or electronic circuitry, which is operated by a software and/or firmware application executed by a processor. In such a case, the processor can be internal and/or external to the apparatus and can execute at least a part of the software and/or firmware application. As yet another example, a component can be an apparatus that provides specific functionality through electronic components without mechanical parts, where the electronic components can include a processor and/or other means to execute software and/or firmware that confers at least in part the functionality of the electronic components. In an aspect, a component can emulate an electronic component via a virtual machine, e.g., within a cloud computing system.

As it is employed in the subject specification, the term “processor” can refer to any computing processing unit and/or device comprising, but not limited to, single-core processors; single-processors with software multithread execution capability; multi-core processors; multi-core processors with software multithread execution capability; multi-core processors with hardware multithread technology; parallel platforms; and/or parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, and/or any combination thereof designed to perform the functions described herein. Further, processors can exploit nano-scale architectures such as, but not limited to, molecular based transistors, switches and/or gates, in order to optimize space usage and/or to enhance performance of related equipment. A combination of computing processing units can implement a processor.

Herein, terms such as “store,” “storage,” “data store,” data storage,” “database,” and any other information storage component relevant to operation and functionality of a component refer to “memory components,” entities embodied in a “memory,” or components comprising a memory. Memory and/or memory components described herein can be either volatile memory or nonvolatile memory or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, and/or nonvolatile random access memory (RAM) (e.g., ferroelectric RAM (FeRAM). Volatile memory can include RAM, which can function as external cache memory, for example. By way of illustration and not limitation, RAM can be available in many forms such as synchronous RAM (SRAM), dynamic RAM (DRAM), synchronous DRAM (SDRAM), double data rate SDRAM (DDR SDRAM), enhanced SDRAM (ESDRAM), Synch link DRAM (SLDRAM), direct Rambus RAM (DRRAM), direct Rambus dynamic RAM (DRDRAM) and/or Rambus dynamic RAM (RDRAM). Additionally, the described memory components of systems and/or computer-implemented methods herein include, without being limited to including, these and/or any other suitable types of memory.

The embodiments described herein include mere examples of systems and computer-implemented methods. It is, of course, not possible to describe every conceivable combination of components and/or computer-implemented methods for purposes of describing the one or more embodiments, but one of ordinary skill in the art can recognize that many further combinations and/or permutations of the one or more embodiments are possible. Furthermore, to the extent that the terms “includes,” “has,” “possesses,” and the like are used in the detailed description, claims, appendices and/or drawings such terms are intended to be inclusive in a manner similar to the term “comprising” is interpreted when employed as a transitional word in a claim.

Installing a child safety seat correctly is crucial for ensuring your child's safety while traveling in a vehicle. The specific steps may vary depending on the type and model of the child safety seat, as well as the vehicle you are using. By introducing more advanced vehicle interior sensing capabilities by which a vehicle, via the system, will be able to identify different features in the vehicle compartment such as visual appearance, shape, weight, movement, belt payout and other parameters. By use of one or more of these monitored parameters, the vehicle, with the system, may advise the user on safe configuration of, for example, accessories such as child safety seats. By the use of the vehicle sensing capabilities and learned recognition of a correct installation, the vehicle can advise the user of potential erroneous child seat installations. There are recommendations for how a child safety seat should be installed. The recommendations can vary both in regards to child safety seat brand, as well as car brand.

Several parameters are to be considered when judging whether a child safety seat is installed properly, some examples being: (i) The belt is correctly routed around the child safety seat; (ii) The belt is sufficiently tightened around the child safety seat; (iii) The child safety seat is not installed crooked; (iv) The child safety seat is properly connected via the international standard for attachment points for child safety seats in passenger cars (ISOFIX) connections; (v) There is sufficient distance between child safety seat and instrument panel or front seats; (vi) The front brace is standing on the floor properly.

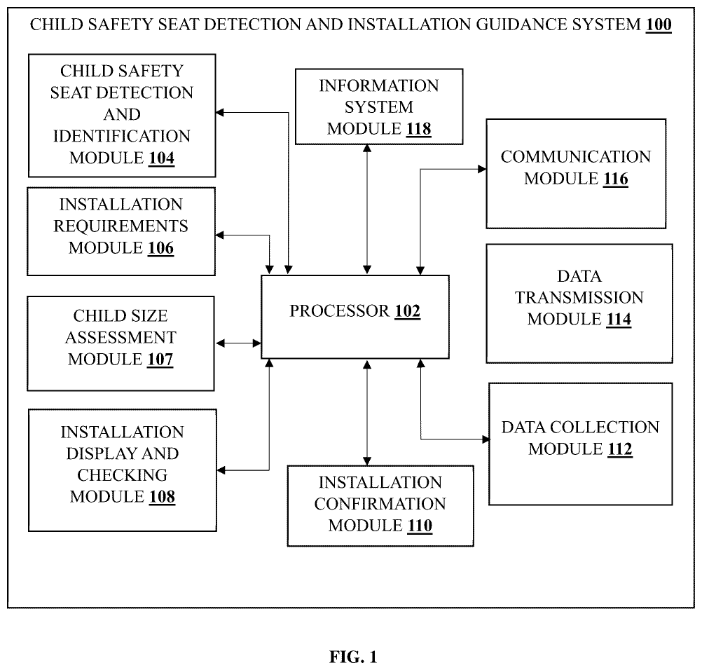

FIG. 1 shows a child safety seat detection and installation guidance system 100 according to an embodiment. The child safety seat detection and installation guidance system 100 comprises, a processor 102, a child safety seat detection and identification module 104, an installation requirements module 106, a child size assessment module 107, an installation display and checking module 108, an installation confirmation module 110, a data collection module 112, a data transmission module 114, a communication module 116, and an information system module 118.

When a user opens the door and places a child safety seat, the child safety seat detection and identification module 104 of the system recognizes the type of child safety seat that user is installing. The child safety seat detection and identification module 104 may be installed within the vehicle that is configured to determine the type of child safety seat. The child safety seat detection and identification module 104 of the system may interface with one or more sensors comprising a camera sensor, LIDAR sensors, radar, infrared sensor, and other sensors that are inside the vehicle, that are configured to recognize the type of a child safety seat that is being attached.

Child safety seats come in various types, each designed to cater to different age groups and developmental stages of children. The child safety seat detection and identification module 104 may recognize whether the child safety seat is an infant car seat, a convertible car seat, a booster seat, a combination seat, a 3-in-1, or all-in-one seat. Further, the child safety seat detection and identification module 104 may recognize a model, make, and retrieve information about a year of manufacture, a size, a shape, and a weight of the car seat from a database or identify directly from the child safety seat.

According to an embodiment of the system, the vehicle comprises a Radio Frequency Identification tag reader operable to recognize an object based on the Radio Frequency Identification tag. The system recognizes the child safety seat via a Radio Frequency Identification tag attached to the child safety seat. The system identifies the child safety seat by detecting a shape of the child safety seat via a camera; and by detecting a weight of the child safety seat via a weight sensor installed in the corresponding seat of the vehicle. According to an embodiment of the system, the sensor comprises one or more of a camera, a LiDAR, a radar, and a Radio Frequency Identification sensor.

Once the system determines the type of child safety seat that is being attached, the system is configured to access an installation manual via an installation requirements module 106. Accessing the manual may be according to the following embodiments:
(i). The vehicle in which the child safety seat is being installed may recognize the make and model of the child safety seat and access the relevant installation manual from a website, for example manufacturer's website.
(ii). Radio Frequency Identification (RFID) technology or a Quick Response code (QR code) technology may be used to access a link via Uniform Resource Locator (URL) from which a manual may be accessed.
(iii). An installation manual may have been embedded in the child safety seat via a chip which can be accessed via a connector, for example, a wired connector, a near field communication, and Bluetooth®.

Installation requirements module 106 may further comprise instructions on child placement and harnessing. Further, the manual may comprise height and weight recommendations. It may further comprise adjustable parts of the child safety seat and adjustment recommendations based on the weight and height of the child safety seat. It may further comprise age recommendations and adjustments based on the age of the child.

In an embodiment, the system recognizes the type of child safety seat that is being installed and extracts all the information about that child safety seat. Information extracted may be through cameras, connectors, RFID, near field communications, Bluetooth®, and a combination thereof. In an embodiment, the child safety seat communicates to the vehicle regarding the type of child safety seat, its shape, size, model, safety features, year of manufacture, installation procedure, usage limits, etc.

According to an embodiment of the system, the system recognizes the child safety seat via a Radio Frequency Identification tag attached to the child safety seat. According to an embodiment of the system, the system identifies the child safety seat, by detecting a shape of the child safety seat via a camera, and by detecting the weight of the child safety seat via weight sensors installed in the corresponding seat of the vehicle.

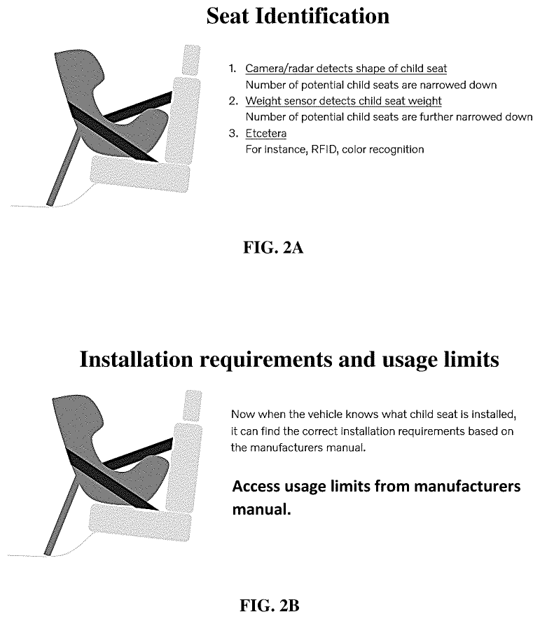

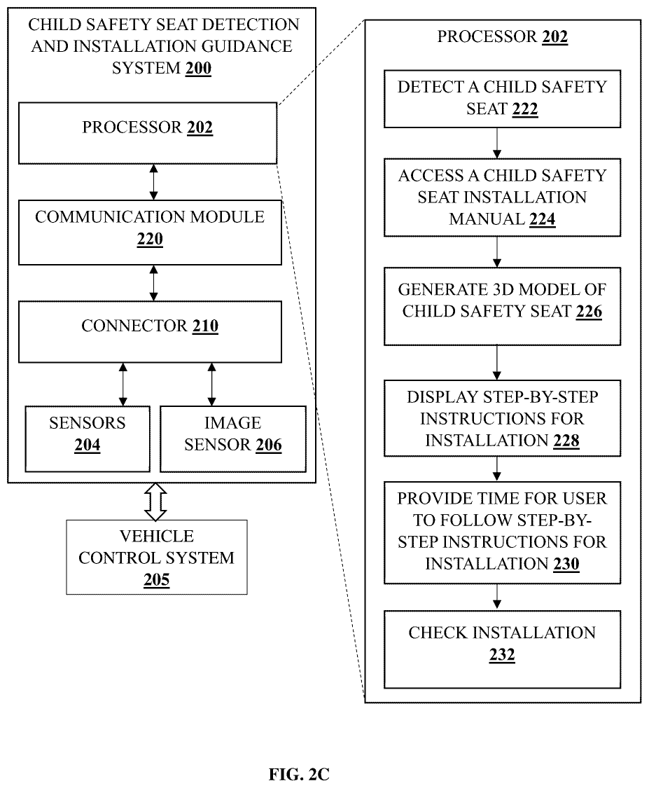

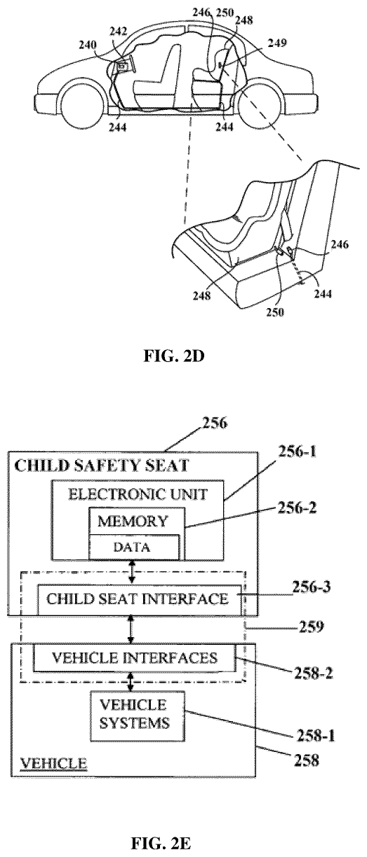

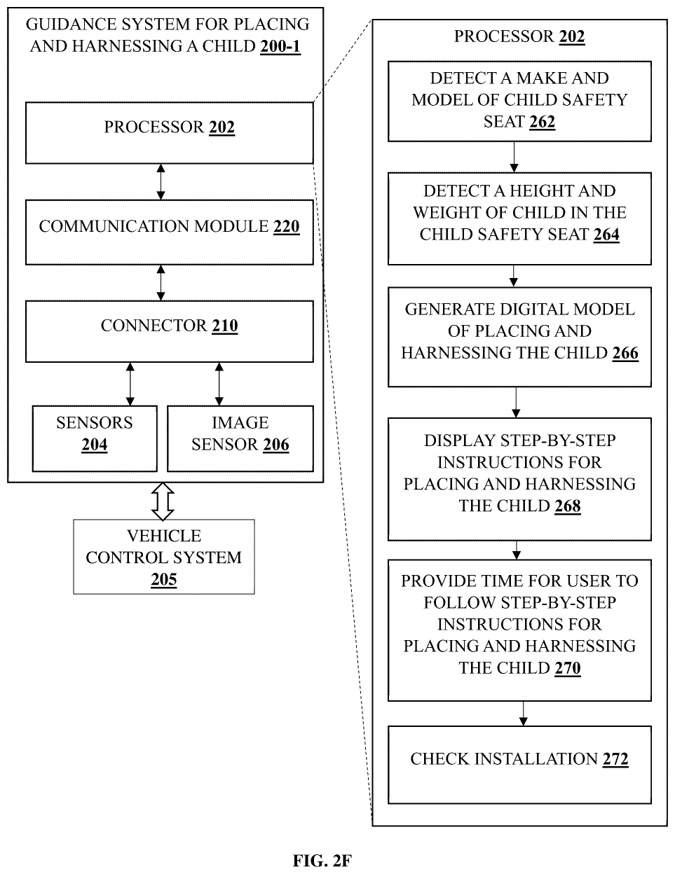

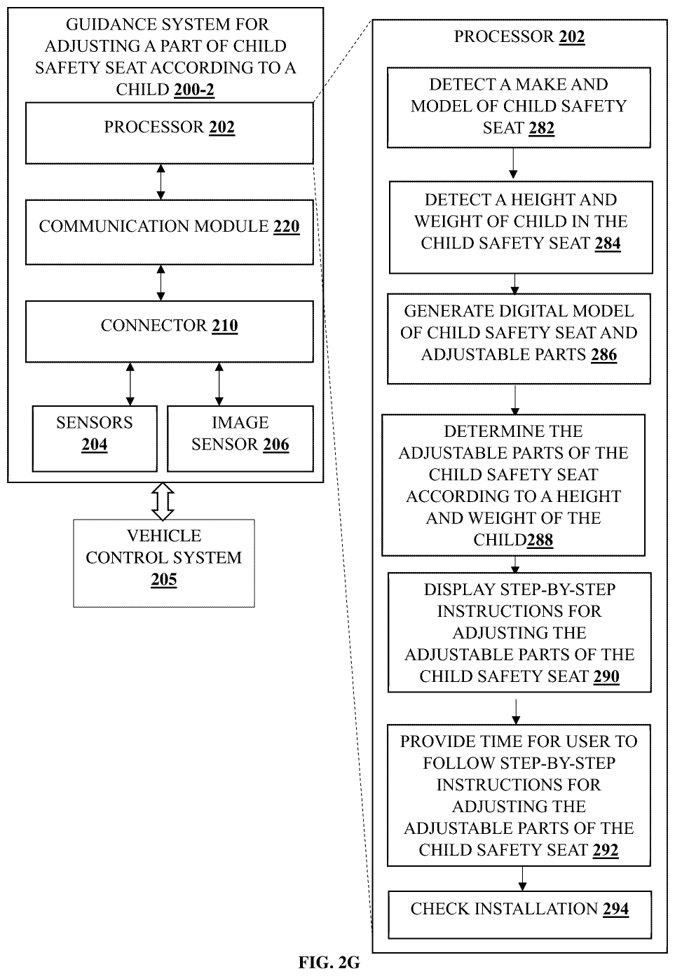

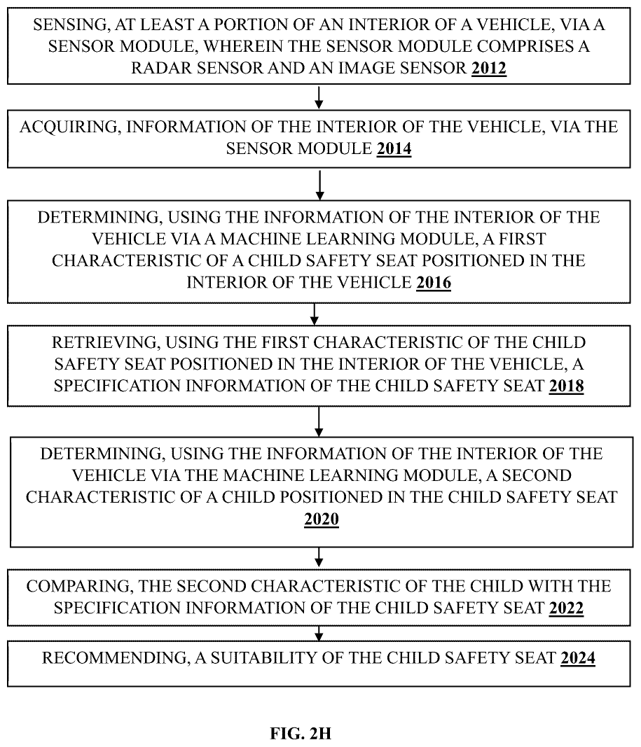

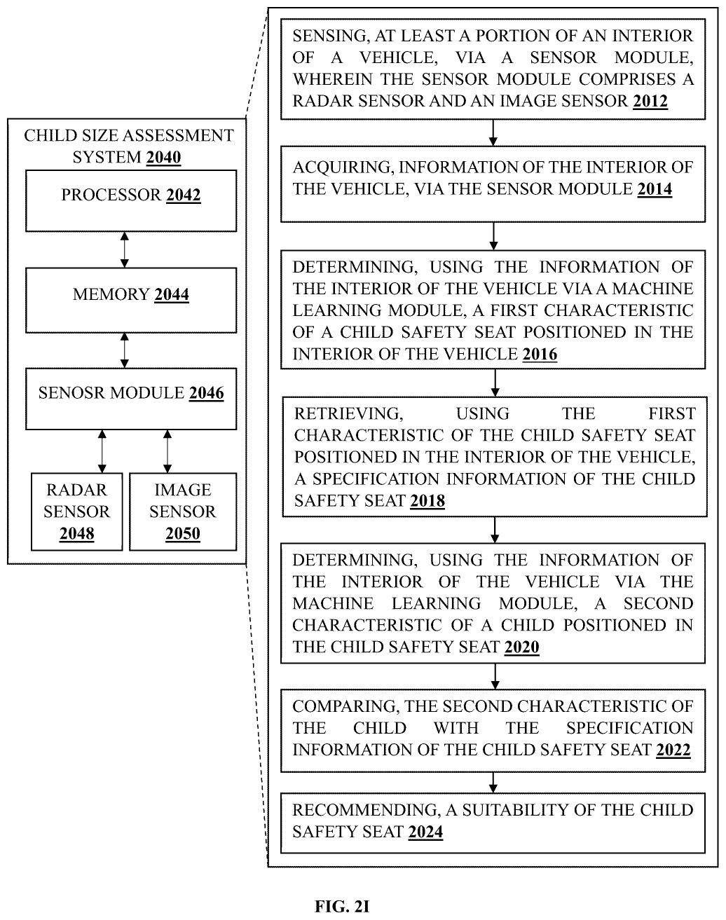

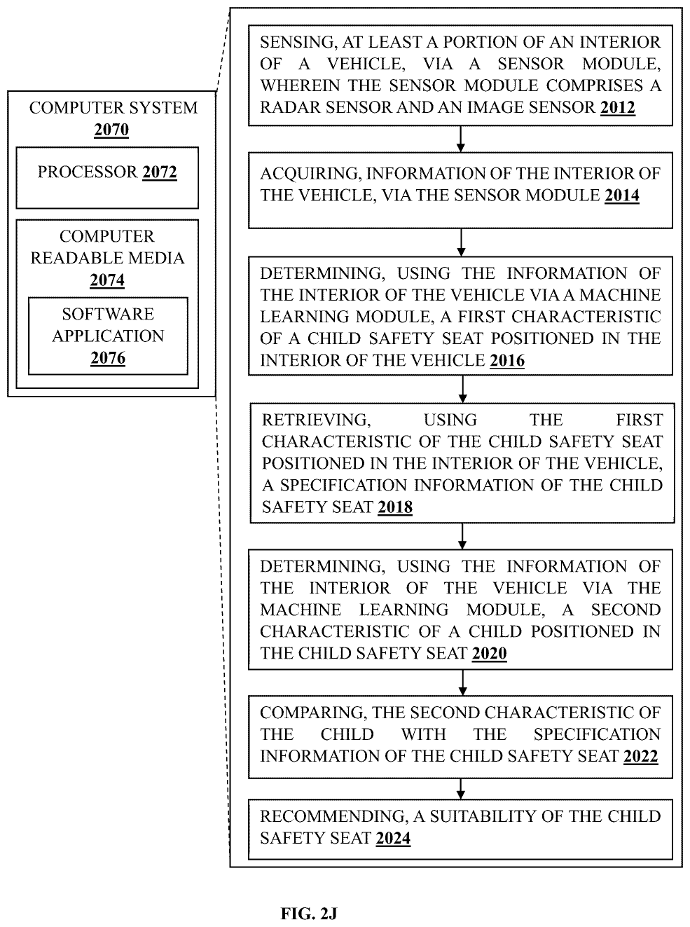

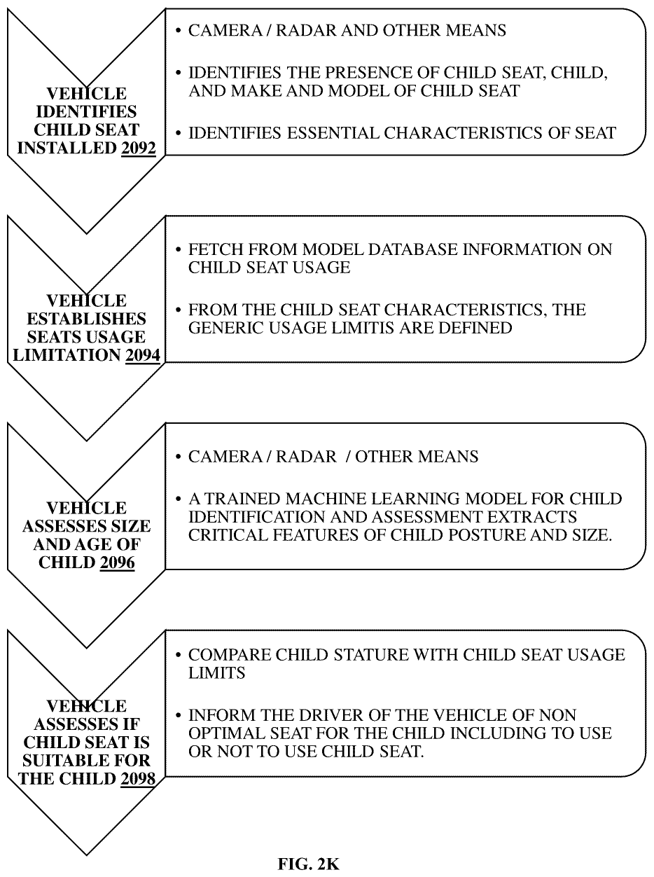

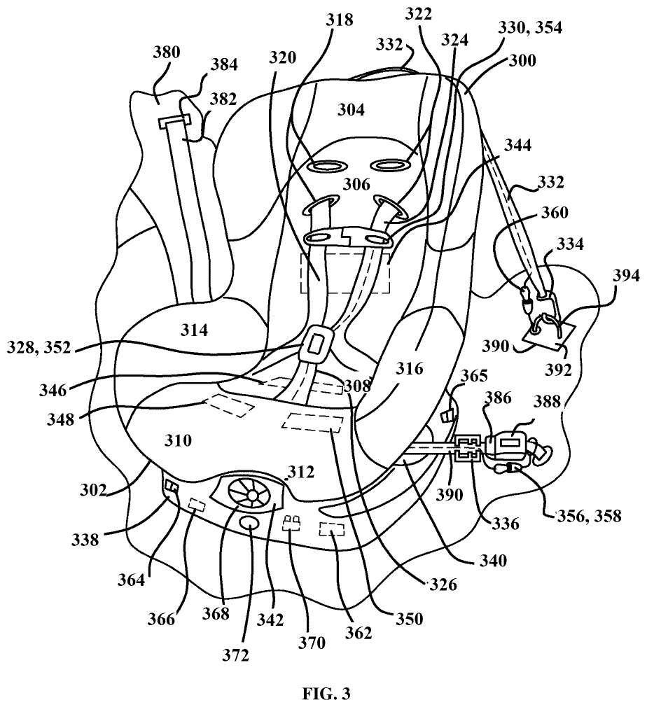

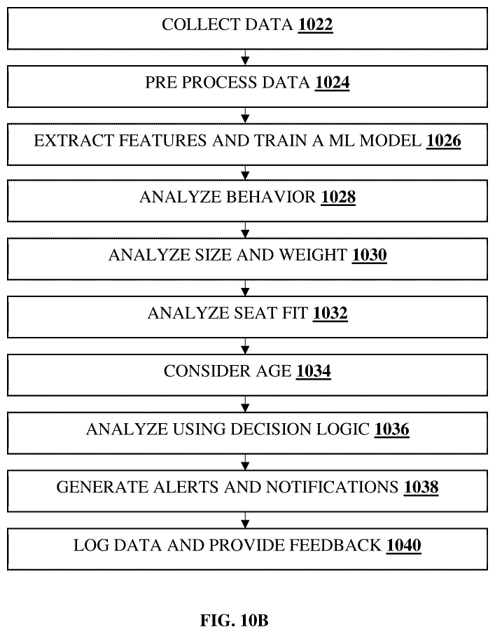

The system comprises a child size assessment module 107. The child size assessment module comprises sensors for interior sensing systems such as camera, radar, and other sensors to identify the type of child seat, child seat installation setup, and child seated. By the introduction of higher sensing capabilities in a vehicle compartment, the vehicle can better understand seated occupants' presence and stature. The child size assessment module 107 comprises a method and a system for child size assessment as shown in FIG. 2H, FIG. 2I, FIG. 2J. Further it comprises steps involved in child seat suitability using AI/ML models as explained in FIG. 2K and FIG. 10B. The components of the module and the method flow charts for FIG. 2H, FIG. 2I, FIG. 2J, FIG. 2K, and FIG. 10B are explained in detail herein somewhere else. By comparing the child seat recommended limits from the child safety seat manual and the child size, weight, age, fit, and behavior of the child in the child safety seats, the system can judge if the child safety seat is suitable for the child. If not, the system can advise the care holder of the child to review the child seat use. The system uses the sensing capabilities in the vehicle interior to identify the child seat and the child, to improve the safety for children traveling in a vehicle.

Once the system determines the type of child safety seat that is being attached, the system assists the user via installation display and checking module 108. The child safety seat may comprise a plurality of sensors which are coupled with the vehicle and provide feedback. The system may be configured to generate a digital model of the child safety seat and display step-by-step installation instructions by using the digital model. The digital model may be either generated by the system itself or may be accessed via the instruction manual. According to an embodiment of the system, the system finds an installation requirement based on a manufacturer's manual. According to an embodiment of the system, the manufacturer's manual is accessed via a local memory. According to an embodiment of the system, the manufacturer's manual is accessed via a network. Instructions may be a video or animation, a text, and an audio or a combination of all. In an embodiment, relevant portions where focus is required may be highlighted. For example, when a belt must pass through a certain portion of the child car seat, then the relevant portion on the child safety seat digital model may be highlighted and/or animated. Further the system may also highlight the relevant portion on the child safety seat itself. In an embodiment, the child safety seat comprises sensors and indicators coupled to the digital model of the child car seat such that when the digital model indicates a certain position on the child safety seat, an indicator may glow that portion on the child safety seat. According to an embodiment of the system, the step-by-step instructions comprise one or more of a text, a figure, an audio, and an animation.

According to an embodiment of the system, the digital model comprises a 3D image generated by capturing a plurality of 2D images of the child safety seat from a plurality of angles. According to an embodiment of the system, the system is operable to capture, via a camera, an interior of the vehicle. According to an embodiment of the system, the information system presents the step-by-step instructions in a user-friendly format. According to an embodiment of the system, the processor is further operable to receive feedback from the user regarding the quality and effectiveness of the step-by-step instructions presented on the information system, and to update the step-by-step instructions based on the feedback received. According to an embodiment of the system, the processor is operable to adjust a presentation of the step-by-step instructions and diagrams based on a proficiency level of the user, wherein the proficiency level is measured based on a time taken and an accuracy of the installation for each step of the installation.

According to an embodiment of the system, the system further comprises a memory module operatively connected to the processor, wherein the memory module stores a plurality of child safety seat installation procedures and protocols, and wherein the processor selects and displays a procedure and protocol based on the make and the model of the child safety seat. According to an embodiment of the system, the processor is operable to display the step-by-step instructions in multiple languages. According to an embodiment of the system, the processor is operable to present instructional videos and animations to enhance understanding of the user for installing the child safety seat.

A system for guiding installation of child safety seats includes sensors mounted on the child safety seat for detecting a progress in a plurality of sequential installation steps. The installation steps are required for installing the car safety seat on a vehicle seat. The car safety seat further includes light emitting elements arranged at various locations, each location corresponding to one of the installation steps. A processor is adapted to execute code instructions for, in response to outputs of the sensors, identifying completion of a current installation step. The processor is further adapted to instruct a suitable light emitting element to indicate a location corresponding to an installation step following the current installation step. The sensors may be coupled with the infotainment system of the vehicle and are correlated with the instructions for installation of the child safety seat. In an embodiment, the child safety seat may be referring to a combination of a child safety seat base and the child safety seat and are addressed independently for installation purposes via various sensors on each of the components.

Installation display and checking module 108, may further comprise displaying of instruction for placing and harnessing the child, checking for proper placing, and harnessing the child, a display for guiding the user for adjusting various parts of the child safety seat.

The system may display a step and provide time for the user to take action to complete the step. The system may provide a signal, for example, an alert and/or a certain message, to indicate whether the step is completed. For example, a one beep means the step is properly executed. Two beeps may mean the step is not complete or not executed properly. For example, the seat may not have been attached the way it is supposed to be. In an embodiment, the signal may be a visual cue, an audio cue, a tactical cue, and a combination thereof. The system may then proceed to provide the next step of the step-by-step instructions. Once the user completes a step successfully and correctly, then the system will proceed to the next step. If the user is not able to finish the step completely and accurately, or if the user is taking more than a predetermined time, the system may assist with additional information. During the installation process, at each step, sensors collect data, and each step and sub-step is time stamped. According to an embodiment of the system, the processor is operable to provide a time-stamped record of the installation for each step, which can be accessed and reviewed at a future time by one or more of the users and a manufacturer of the child safety seat.

An alert is provided whether the seat is attached or not properly attached. Once the seat is properly attached then the system guides the seat for attachment points and a routing of the seat belt. The system then guides the points by highlighting animation on the digital model on the routing of the seat belt and checks whether the seat belt has gone through the designated points correctly. In an embodiment, various indicators are placed at the points where the seat belt must be routed. These points on the child safety seat may glow or provide a light to attract user attention. In an embodiment the indication may be a combination of a light cue, a sound cue, and a tactile cue. In an embodiment, one or more sensors are provided on the vehicle seat belt to provide check points for example on routing, crookedness, straightness, etc.

There are typically two types of attachment points in most vehicles:

Lower Anchors: These are metal bars or loops located in the vehicle's rear seat crease, between the seat cushion and seatback. They are specifically designed to anchor the lower attachments of a child safety seat. The lower attachments of the child seat are usually equipped with connectors that can be easily attached to these lower anchor points.

Tether Anchors: Tether anchors are metal bars, loops, or hooks located either on the vehicle's rear shelf or behind the rear seat. They are used to secure the upper tether strap of the child safety seat. The tether strap is connected to the top portion of the child seat and provides additional stability by limiting the forward movement of the seat in the event of a collision.

ISOFIX is an international standard for the attachment of child safety seats in vehicles. It is also known as LATCH (Lower Anchors and Tethers for Children) in the United States. According to an embodiment of the system, the attachment point comprises a standard belonging to one or more of an International Standard for attachment points for child safety seats (ISOFIX), Lower Anchors and Tethers for Children (LATCH), Lower Universal Anchorage System (LUAS), CANADAFIX (CANFIX), and Universal Child Safety Seat System (UCSSS).

Data from the sensors on the child safety seat, vehicle seat belt, attachment points are collected via a connector which would connect to the vehicle system to confirm that the seat is attached properly, the seat belt is straight and not crooked, its tautness, etc. When the user wraps the vehicle seat belt around the child safety seat, the system provides the user feedback that informs whether the vehicle seat belt is routed properly. In an embodiment, a proper routing is based on the child safety seat model, size, and shape that is recognized by the system. The system measures the tightness (tautness) of the vehicle seat belt. In an embodiment, if the vehicle seat belt is loose, the system may provide feedback to the user to tighten the vehicle seat belt. In an embodiment, the system automatically adjusts the seat belt for required tautness. In an embodiment, if the system fails to adjust the vehicle seat belt to required tautness, the system provides feedback to the user of the same and informs the user to adjust the vehicle seat belt for the required tautness. In an embodiment, the required tautness is based on manufacturer's specifications. In an embodiment, the required tautness may be based on data collected over time, and the seat movement may be based on the vehicle seat belt tautness. The system learns the correlation among vehicle seat belt tautness and the child safety seat movement over time based on safety, comfort, child's weight, height, and the child's behavior. The system may then recommend a correct level of tautness based on the learned data. In an embodiment, the system may learn using artificial intelligence models. In an embodiment, the system checks for a crookedness of the vehicle seat belt. The system determines installation requirements based on safety, comfort, the child's weight and height and the behavior of the child.

In an embodiment, based on the child's height, the system determines a required distance from a seat that is in front of the child safety seat and may recommends an adjustment of the seat. In an embodiment, the system automatically adjusts for a required distance from a seat that is in front of the child safety seat. The system may assume or recommend a height limit of the person that may be sitting in the seat that is in front of the child safety seat. In an embodiment, the system can recommend the child safety seat be installed in another vehicle seat. According to an embodiment of the system, the object in front of the child safety seat comprises an instrument panel. According to an embodiment of the system, the object in front of the child safety seat comprises a seat of the vehicle.

In an embodiment, based on the child's weight, height, and behavior, the system may recommend that a change in the child safety seat is required. In an embodiment, based on the child's weight, height, and behavior, the system may recommend a tautness of the vehicle seat belt. In an embodiment, based on the child's weight, height, and behavior, the system may recommend a required distance from a seat that is in front of the child safety seat. In an embodiment, the recommendation may consider the past data collected over a period, wherein the data may comprise a first data while the vehicle is in motion, a second data while the vehicle is not in motion, a third data when a vehicle encounters a crash. In an embodiment, the system is configured to recommend, even when some data points or data sets are missing.

In an embodiment, the child safety seat is configured for communication. In an embodiment, the child safety seat or the vehicle is configured for performing routing of the seat belt and adjustment of the seat belt. For example, the vehicle, having a physical connector or Bluetooth® connector or near field connector, knows as soon as the user places the child safety seat, the vehicle determines the kind of child safety seat, i.e., make, model, year of manufacture, size, shape, usage limits, etc., and what features the child safety seat has. Further, the system may know whether the child safety seat is properly installed by the user or not. The system will alert until the child safety seat is properly placed. The system continues to monitor the child safety seat via sensors.

The system guides the user on how to place the child safety seat and adjust properly. The systems may check various parameters after the child safety seat is placed to determine whether it is placed straight or crooked. The system may then provide feedback if the placement is proper and correct. In an embodiment, the confirmation is displayed on the infotainment system of the vehicle. A visual effect may be displayed on the infotainment on whether the installation is correct or where it is installed in the wrong manner and how to correct the same. When the installation is performed correctly, the driver or the user, may get feedback that the installation is correct via an audio beep, or if the installation is incorrect, a voice message may state “installation is not done properly, the vehicle seat belt is crooked, please check” and may animate the digital model to highlight the portions providing focus on where the user needs to check.

The system may perform various checks. For example: Is the child safety seat attached properly? Did the vehicle seat belt route through the child safety seat properly? The checks may vary based on the type of seat, for example, a booster seat, a regular seat, forward-installation, backward installation, forward facing seat, rear facing seat, etc.

In an embodiment, the system may check for various parameters when the child gets seated into the child safety seat. In an embodiment the system checks for more than one child safety seat being installed. In an embodiment, the system would check for more than one child using the same child safety seat. In an embodiment, the system may adapt the installation requirements based on the child's weight and height. The system may adapt to it, meaning that a belt tautness or tautness for a 40-pound child may be different from the child who is 60 pounds. Similarly, children of different heights require different tautness. In an embodiment, sensors in the vehicle and the child safety seat are configured to detect the child height and weight. In another embodiment, the user may be able to enter the height and weight via the infotainment system. In an embodiment, the system monitors the child's movement and behavior and may consider the child's movement and behavior in the child safety seat while determining the tautness requirements for the vehicle seat belt. Installation of a child safety seat may be different if a child is attempting to come out of the seat. Some children may have the strength, dexterity, or curiosity to unbuckle or loosen the harness straps, making it necessary to take additional precautions during the installation process. In an embodiment, the system guides the user to properly adjust and snugly secure the harness straps to the necessary position of the child's shoulders and torso. It may help the user to check for proper tension to make sure the straps are in the correct slots according to the child safety seat's instructions to help minimize the child's ability to wiggle out of the child safety seat or loosen the harness. Prior behavior of the child is monitored and factored in to determine an installation requirement or a parameter in the installation, such as tautness of the vehicle seat belt. The system may output graphical, visual, and audio feedback based on accuracy of the installation.

Installation confirmation module 110, confirms whether all the steps of the installation are completed properly. It may generate a summary report with a checklist and time taken for each step. In an embodiment, the system checks if the child safety seat is securely installed by giving it a firm tug at the base and checking for any excessive movement. In an embodiment, the tug or the force is generated by actuators installed within the child safety seat or outside of the child safety seat within the vehicle. In another embodiment, the user may be guided to apply force at different points to check for the movement of the child safety seat. The system may also check and ensure that all straps, buckles, and attachments are properly fastened and adjusted.

According to an embodiment of the system, the processor generates a summary report of the placing and harnessing of the child in the child safety seat, wherein the summary comprises a checklist of the placing and harnessing of the child in the child safety seat and corresponding values for the checklist prepared from a real-time data of the placing and harnessing of the child in the child safety seat sensed via a first plurality of sensors of the child safety seat and a second plurality of sensors of the vehicle. According to an embodiment of the system, the processor generates a summary report of the adjustment of the adjustable parts of the child safety seat according to a height and a weight of the child.

In an embodiment, the child seat detection and installation system are coupled with the ignition system of the vehicle. The system may allow the driver to start the vehicle when it determines the installation is proper. The indication may be via a light in the key chain or near or around the ignition lock.

Data, from all the steps and tests of installation, may be collected and stored for later use by the data collection module 112. In an embodiment, the data may be uploaded to a cloud or stored locally. The system utilizes data transmission module 114 and communication module 116 for transmitting and communicating data from one component to another component or from one system to another system either internally or externally. Such data collected from a plurality of vehicles and child safety seats, by various users, may be collected on a cloud (a network) and analyzed for including but not limited to i) for understanding which steps needed more time ii) which steps were mostly performed wrong and iii) which steps were easy iv) which steps were taken in less time than a predetermined average time/estimated time v) wrong installations, etc. The data may further be used for i) improving the step-by-step instructions ii) sharing with manufacturers' for improving designs and sensor locations iii) classifying users into groups and providing various recommendations.

In an embodiment, the child safety seat may be checked for registration with the manufacturer. The system may connect with the manufacturer's website, check for any recall notices, and update the user about the recent information on recalls, upgrades and any other relevant information on safety related to the child safety seat.

The system further comprises the Information system module 118 comprising interconnected components, including hardware, software, databases, networks, and people that work together to collect, store, process, transmit, and retrieve information in an organized and meaningful way. The information module involves the use of technology, algorithms, processes, and resources to manage data and support decision-making, feedback, coordination, control, and analysis. An infotainment system may be considered a type of information system. An infotainment system combines information and entertainment features within a vehicle or other consumer electronic devices. It typically includes components such as a display screen, audio system, navigation system, and connectivity features. The display screen of the infotainment system may be used to show to the user instructions and how to execute the instructions for installing a child safety seat. It may comprise animations, voice commands, text, pictures, and combinations thereof. In an embodiment the system is enabled to take feedback from the user. In another embodiment, the infotainment system screen may be mirrored to a portable device, such as a mobile phone so that the user may place the device at a point of view and convenience to be able to install the seat and simultaneously check the device's screen.

In an embodiment, it is a method, where the user opens the vehicle door and puts the child safety seat in the vehicle. The vehicle starts monitoring and determining the type of child safety seat. Once the system determines the type of child safety seat, and the specifications about the child safety seat, it starts gathering all that information on where the car seat is to be placed. Once the child safety seat is placed, the system may generate a graphic on the infotainment that is based on the type and model of the child safety seat. The infotainment system provides a 3D model of the child safety seat. According to an embodiment of the system, the processor is operable to display the digital model as a 3D model of the child safety seat to assist the user in performing the installation.

The system then monitors the parameters of installation and provides feedback to the user via a beeping, a light, a vibration or any indicator or combination of indicators to let the user know the installation accuracy. The user may get an indication whether the seat is properly placed, crooked, sideways, etc. The user may be guided to route the vehicle seat belt and click and secure the child safety seat. The system will analyze through and determine whether the user has properly routed the vehicle seat belt and clicked and secured it. Once the system determines the step was properly executed, the user may further be guided to secure the seat belt to a required tautness. The system may determine that the seat belt tautness is proper, based on prior knowledge acquired through the sensors over a period and the manufacturer's specification. When the child safety seat is being installed for the first time, the system may not have any information, and may base the determination of tautness of the seat belt on the weight of the child or might determine as soon as the user puts the child in the child safety seat. In an embodiment, the system may re-assess based on the child's weight, the seat belt tautness, the placement of the seat, did the child safety seat get moved, and then continuously monitor the parameters. In an embodiment, the system may automatically adjust the tautness of the seat belt, based on whether the child keeps moving or not, based on the driving path and behavior, based on whether the child safety seat is slipping.

By the capabilities offered by modern vehicles, the interior sensing of the vehicle will be much more mature. By combining the sensing capabilities from, for instance, shape recognizing camera/radar; weight sensors in seat bolsters; belt payout measurement; force measurement; RFID tags, etc., the vehicle will be able to detect the make and model of a child safety seat, and whether it is correctly installed or not. If it is judged to be installed properly, the car will give the driver a signal that everything is in order. If not, the car will inform the driver that the child safety seat may not be properly installed and tell the driver what part of the seat installation needs attention. According to an embodiment of the system, the system comprises a computer vision module comprising a shape recognizing camera.

In an embodiment a method comprising a sequence of how the interior sensing could help the driver to detect if the child safety seat is properly installed are:

Child Safety Seat Detection and Identification Module: FIG. 2A shows an example of Child Safety Seat Identification steps according to an embodiment. The Child Safety Seat Detection and Identification Module comprises a camera or radar, a weight sensor, wherein the camera and the radar is configured to
(i). Detect a shape of the child safety seat based on comparison and analysis of the shape of the child safety seat in question and the potential child safety seat shapes.
(ii). Allow one or more weight sensors to detect the questioning child seat weight and then narrow down the match with the potential child safety seats.
(iii). Use RFID and color recognition to narrow down the potential matches and correctly identify a child safety seat model, make, size, shape, and year of manufacture.

Installation Requirements Module: FIG. 2B shows an example for accessing Installation Requirements and usage limits according to an embodiment. When the vehicle identifies the child safety seat that is being installed, it can find the correct installation requirements based on the manufacturer's manual. Further it can also access the manufacturers specification on usage limits. Manufacturers of child safety seats specify usage limits that include age, weight, height, harness height, harness tightness, expiration date, proper installation, recommended position (rear-facing, forward-facing, or booster), discouragement of certain accessories, and replacement after a crash. These guidelines are crucial for ensuring the safety and proper use of the car seat, and caregivers should adhere to them diligently to protect children during car journeys. Access can be via vehicle connecting to the manufacturer's website to access the manual related to the model. It may be accessing a database where various manuals are stored, and the systems locate the correct one and access the same from the database. Databases could be either available locally or via cloud. According to an embodiment of the system, the specification information of the child safety seat comprises one or more of type of the child safety seat, a positioning of the child safety seat, an orientation of the child safety seat, a feature of the child safety seat, a height limit, a weight limit, an age limit; and wherein the orientation of the child safety seat comprises one of a rear facing child safety seat, a front facing child safety seat, a convertible child safety seat, and an all-in-one child safety seat.

Installation Checking Module: This module depicts checking various aspects of the child safety seat installation according to an embodiment. The Installation Checking Module checks whether the child safety seat has been properly installed: (i) Checks whether the child safety seat is straight. (ii) Checks whether the child safety seat has a proper distance to the front seat or instrumentation panel. (iii) Checks whether the belt routing is proper. (iv) Checks whether the child safety seat front brace is properly adjusted. (v) Performs weight or force checks to determine if the child safety seat is tightly pressed towards the bolster and/or installed using ISO fix connections. (vi) Performs belt payout sensor checks to check if the belt is rolled out to the desired length and tautness.

Installation Confirmation Module: The module depicts the Installation Confirmation Module providing a signal to the driver regarding child safety seat installation according to an embodiment. The Installation Confirmation Module confirms that all requirements are followed correctly or not. If all requirements are followed correctly then the driver gets a green light to start the vehicle. In case the vehicle cannot confirm that all the requirements are followed correctly, the vehicle may inform the driver about what areas need special attention before it is OK to start the vehicle.

FIG. 2C shows a block diagram of a system executing a method for child safety seat detection and installation guidance according to an embodiment. The child safety seat detection and installation guidance system 200 may comprise a processor 202, sensors 204, and an image sensor 206. In an embodiment, the processor 202 may receive information from sensors 204 and image sensors 206 to determine a location of the child safety seat. Further, the system analyzes the sensor data. Sensor data may include, but not limited to, a size, a shape, a safety feature, a model, a make, a year of manufacture. For example, an optical sensor can view a vehicle seat within its field of vision and/or an electromagnetic field sensor can determine that there is an ElectroMagnetic Field (EMF) emanating from a location in the vehicle, then the processor 202 may determine that another EMF signal is originating inside or nearby to the vehicle, from the child's safety seat, emanating the same.

In an embodiment, the system may establish a communication with the child safety seat, and the child safety seat may transmit signals containing information related to the child safety seat. The processor 202 detects a type of child safety seat using an image sensor 206 and one or more sensors 204 or receives data of child safety seat through the connector 210. The system may communicate with the child safety seat and the vehicle control system 205 through the communication module 220 via the connector 210.

In an embodiment, the processor 202 is configured to detect a child safety seat at step 222. A start signal is sent from the system to the child safety seat. If the child safety seat is connected to the system through a connector, an acknowledgement is received from the child safety seat. Upon receiving the acknowledgement from the child safety seat, a secure connection is established between the child safety seat and the system, and a child safety seat installation manual is accessed via the connector, at step 224. The system then receives data of the child safety seat and generates a 3D model of the child safety seat, at step 226. The data may comprise one or more of a type of child safety seat, a make, a model, a year of manufacture, a size, a shape, safety features, a suitable position for installation, forward facing installation or rear facing installation, tautness of the belt, how to check for the correct installation, installation checklists, etc. Once the seat is occupied, data about the child is also monitored and may be received via the connector. The received data may be encoded for a secure data message using a protocol. The system may then generate step-by-step instructions for the installation of the child safety seat, at step 228 on the infotainment system of the vehicle. The system may provide time for the user to follow step-by-step instructions for installation of the child safety seat, at step 230. At step 232, the system may check for the accuracy of installation. In an embodiment, the check is performed at each step and after all the steps. A first signal is generated for each step to indicate whether the step is completed accurately or not. Similarly a second signal is generated after completing all the steps to indicate whether the process is completed accurately or not. In an embodiment, the data collected from sensors during installation may be transmitted in real time via the communication module to an application installed in a central server, thereon, the application is configured to receive the data from the central server. An embodiment relates to a system, wherein a system is configured to be a component of a vehicle.

The system comprises a sensor 204, an image sensor 206, a connector 210, a communication module and a processor 202. The processor 202 is configured to detect a child safety seat in the vehicle via the connector 210, establish a secure connection with the child safety seat through the connector 210, receive a first data of the vehicle from the sensor 204, receive a second data from the image sensor 206, process the first data and the second data, and notify a condition of the child safety seat installation through the communication module. The vehicle seat comprises at least one of a normal car seat, a child car seat and a pet car seat. The vehicle seat comprises at least one of a front facing car seat, rear facing car seat and a booster seat. The system comprises a plurality of sensors in the vehicle. The sensor 204 comprises at least one of a seat belt sensor, a position sensor, a pressure sensor, a liquid detection sensor, weight sensor, an infrared sensor, an optical sensor, a comfort detection sensor, a moisture sensor, a temperature sensor, and an audio sensor. The image sensor 206 comprises an infrared image sensor, and a thermal image sensor. The connector 210 comprises at least one of a wired or a wireless connector.

According to an embodiment of the system, the child safety seat comprises a plurality of sensors, wherein the plurality of sensors comprises one or more of a seat belt sensor, a position sensor, a pressure sensor, a liquid detection sensor, a weight sensor, an infrared sensor, an optical sensor, a comfort detection sensor, a moisture sensor, a temperature sensor, and an audio sensor. According to an embodiment of the system, the vehicle comprises a plurality of sensors, wherein the plurality of sensors comprises a seat belt sensor, a position sensor, a pressure sensor, a liquid detection sensor, weight sensor, an infrared sensor, an optical sensor, a comfort detection sensor, a moisture sensor, a temperature sensor, and an audio sensor. According to an embodiment of the system, a belt of a seat in the vehicle comprises a plurality of sensors, wherein the plurality of sensors comprises a position sensor, a pressure sensor, a liquid detection sensor, a weight sensor, an infrared sensor, an optical sensor, a comfort detection sensor, a moisture sensor, a temperature sensor, and an audio sensor. According to an embodiment of the system, a belt of the child safety seat comprises a plurality of sensors, wherein the plurality of sensors comprises a position sensor, a pressure sensor, a liquid detection sensor, a weight sensor, an infrared sensor, an optical sensor, a comfort detection sensor, a moisture sensor, a temperature sensor, and an audio sensor.

In an embodiment, the second data comprises an image of the child safety seat's position, a second position of the occupant on the child safety seat, and a size of the child safety seat according to the height and weight of the occupant. The first data comprises at least one of a first position of the child safety seat, a second position of the occupant on the vehicle seat, a weight of the occupant, a height of the occupant, and a pressure on the seat belt.

The child safety seat detection module detects the child safety seat by exchanging handshaking signals to the vehicle seat through a connector 210. A flow chart for detection of the child safety seat comprises a processor sending a start signal through the connector in order to detect a child safety seat; if there is a vehicle seat attached to the connector, the processor may receive an acknowledgement signal from the child safety seat; upon receiving the acknowledgement signal, the processor establishes a secure connection with the child safety seat. The processor may receive a signal at the communication module from the child safety seat. The processor may further automatically determine if the signal originates from inside the vehicle. If the signal does not originate from inside the vehicle, then the processor is configured to reject the signal. If the signal originates from inside the vehicle, the processor communicatively connects the communication module to the child safety seat. Then the processor is configured to receive data and information stored in its memory from the child safety seat. The communication transceivers may create a geo-fence (i.e., a virtual perimeter for a real-world geographic area) around the vehicle that allows the communication system to determine whether received signals are currently originating from inside the vehicle, or within a certain range of the location of the vehicle.

FIG. 2D shows an implementation of a connector for child safety seat detection and installation guidance system according to an embodiment. When a child safety seat is connected into the vehicle, such as a child car seat or a pet car seat, the child safety seat should be detected by the vehicle. Although wireless communication between sensors and an infotainment system 240 of the vehicle are enabled, it is specifically contemplated that a sensor or group of sensors may instead be connected to an infotainment system 240 through a wired connection. For example, in the embodiment depicted in FIG. 2D, the infotainment system 240 is, or is part of, the vehicle infotainment system, or a processor of electronic control unit/s, coupled to the infotainment system 240. In the depicted embodiment, the vehicle may include one or more physical wires 244 with one end connected to the vehicle infotainment system 240 and the other end having a connector 246. At the terminal end of the wire 244, the connector 246 will generally be proximate to an installation point for a child safety seat 248, such as the back row of a sedan, the middle or back row of a minivan, or near a LATCH or other anchoring system. The connector 246 is generally designed for storage in the cavity of the vehicle seat between the upright portion of the seat and the lateral portion. This protects the connector 246 from damage when not in use and is aesthetically desirable, similar to LATCH and other anchoring systems which are also generally hidden in such cavities. The sensors in such an embodiment are similarly wired to a connector 250 of the child safety seat configured for mating with the connector 246 of the vehicle. When the child safety seat 248 is installed in the vehicle, the connector 250 of the child safety seat is plugged into the connector 246 of the vehicle to establish the wired connection between the vehicle system/hub 242 and sensors. This system has the advantage of a more stable and reliable connection than a short-range wireless connection, less likelihood of interference from external sources, and simpler configuration. Further, in an embodiment, wires 244 can power or charge sensors, eliminating the need for a battery as the primary power source.

The central processing hub/the vehicle infotainment system/ECU includes most of the software and programmatic logic of the systems and methods, and will generally be, or be integrated into, a vehicle system, a mobile device, or both. The vehicle infotainment system 240 generally comprises a logic unit or microprocessor, a wireless transmitter/receiver, and may optionally include a non-transitory computer-readable data storage system. This system may be integrated into the vehicle infotainment system. The vehicle infotainment system also generally comprises one or more software components or modules in the form of computer-readable instructions stored on a non-transitory computer-readable memory or storage media. It will be understood by one of ordinary skill in the art that the vehicle infotainment system will generally comprise additional components including, but not necessarily limited to, a bus to move data among components within the vehicle systems. In an embodiment, the wired port is a Universal Serial Board (USB) connector or a proprietary connector 249.

FIG. 2E shows a diagram of an embodiment showing a child safety seat comprising an electronic unit including a memory loaded with child safety seat data, and a child seat interface for communication with the vehicle. The diagram includes chematic illustrations of an embodiment showing an intelligent child safety seat 256 and a vehicle 258. The child safety seat 256 has an electronic unit 256-1 including a memory 256-2 loaded with child safety seat data, and a child seat interface 256-3. In parallel, the vehicle 258 has vehicle systems 258-1 and a vehicle interface 258-2. The electronic unit 256-1, the memory 256-2 with child safety seat data are configured for establishing communication between the child safety seat 256 and the vehicle 258. Both the child seat interface 256-3 and the vehicle interfaces 258-2 form a joint interface 259 that provides mechanical, electrical, electronic, and communication coupling between the electronic unit 256-1 of the child safety seat 256 and the vehicle systems 258-1 operative in the vehicle 258. The term vehicle applies to all means of transportation of passengers. The joint interface 259, framed by a dash-and-dot line in FIG. 2E, refers jointly to both the child seat interface 256-3 and the vehicle interfaces 258-2, which are disposed of in the child safety seat 256 and in the vehicle 258 respectively. Communication between the child seat interface 256-3 and the vehicle interfaces 258-2 may be either wired or wireless. The joint communication interface included in the joint interface 259 may be implemented as a wireless communication interface, or as a wired communication interface, or as an interface having a combination of both wireless and wired communication. The child safety seat 256 may also be configured for independent operation, thus without the support of the vehicle systems. In other words, the child safety seat may be configured for operation dependent on the vehicle, and also independently from the systems operative in the vehicle. The joint interface 259 may include a unidirectional data link, a bi-directional data link, or a combination thereof and is referred to as a connector. The child seat interface 256-3 and the vehicle interface 258-2 may be integrated or embedded as desired. For example, the child seat interface 256-3 may possibly be integrated into, or associated with, the operation of the child-seat mechanical interface which is the mechanical portion of the child seat interface that may include at least one of the mechanical means pertaining to a child restraint system (CRS). The mechanical means of a CRS may include lower attachment assemblies, such as ISOFIX lower rigid attachments, or LATCH lower flexible attachments, and the top tether hardware at the end of the LATCH attachment, such as a top tether hook, push-button connector, or latching device compatible with lower anchorages and top tether anchorage of the vehicle mechanical interfaces. Likewise, the vehicle interfaces 258-2 may possibly be integrated into, or associated with, the operation of the vehicle mechanical interfaces, for example, embedded in the buckle of the vehicle's seat belt. The joint interface 259 automatically communicates signals permitting automatic recognition of the presence of the child safety seat 256 in the vehicle, and allowing for data collection or communication.

One purpose of the joint interface 259 includes the exchange of data between the child safety seat 256 and the vehicle 258, or at least, the emission of data, referred to as identity-data, from the child safety seat 256 to the vehicle systems 258. The term identity-data applies to all data items originating from the electronic unit 256-1 of the child safety seat 256 that are communicated to the vehicle systems 258-1, and to the data items received by the electronic unit 256-1 from the vehicle systems 258-1. As described herein, the identity-data includes at least child safety seat data, but may also include child safety seat data together with data selected alone and in combination from the group consisting of sensor data and device data.

Child Restraint Systems or CRS, are well known in the art of transporting children of different ages. To avoid confusion of terms, the term CRS also applies to “car seat”, “child seat”, “child safety seat”, “child restraint or CR”, and the like. Child restraint systems include, for example, rear-facing restraints (infant-only and convertible), forward-facing restraints (convertible, child seat, combination seat), car beds, harnesses, and boosters (belt-positioning and shield).

The vehicle 258 is fitted with various vehicle systems, possibly controlled by a processor and including a plurality of sensors, operable devices, and alert systems. The sensors of the vehicle systems, may include, for example, sensors configured for the detection of events including seat belts buckling status, ignition switch status and ignition key presence, load on seats, engine Revolutions Per Minute (RPM), gearbox shift position, parking brake status, window and door-lock positions, and smoke detection. These sensors are continuously or intermittently sampled under command of the vehicle systems. The operable devices of the vehicle systems may include many different items, including amongst others: engine ignition switch, ignition key switch retention mechanism, shift-interlock such as brake-to-shift or transmission shift, key-free system, gearbox functioning, seat restraint systems like the seat belt system and the airbag system, power window and door lock modules, devices for releasing the trunk-lid from inside the trunk compartment, central doors-locking devices, power accessories like electrically operated roof-panels, climate control devices, communication systems to establish communication with communication networks, the notification system, the automatic collision notification system, internet communication systems, information systems like the driver message center, navigation systems, driving assisting systems like parking, imaging and observation system, parking aid systems, lane departure and lane change systems, driver condition warning systems like for detecting driver intoxication and driver drowsiness, entertainment devices, information devices like belt reminder systems, occupant classification sensing devices, occupant detection devices, event data recording devices, audio systems, cigarette lighters, internal and external lights, the horn, the burglar alarm, the vehicle immobilizer system, the clock, and the timer. The alert systems of the vehicle systems may include, for example, visual or audible signals, dedicated vehicle warning lights, the interior lights of the vehicle, audible alerts, vocally recorded messages, screen readable messages, screen displayed images, warning labels and messages, operation of the audio system, and prevention of removal of the ignition key. Signals may also include the horn, the exterior lights, the vehicle's burglar alarm siren, and signals received by remote communication systems. Other signals, even though not perceptible by sight or sound, include disabling of the vehicle's door locking mechanism, and the operation of some elements out of the communication unit, such as for emitting signals to one or more remote stations. Other alert signals include tactile signals and vibration emitting signals.

FIG. 2F shows a guidance system for placing and harnessing a child 200-1 according to an embodiment. Proper placement of a child in a child safety seat is crucial for their safety, but several common problems can arise. These include loose harness straps, misplacement of the chest clip, incorrect harness height, improper recline angle, incorrect seat belt routing, using an incompatible seat, and improper installation. To address these issues, it is important to carefully follow the manufacturer's instructions, consult certified child passenger safety technicians, and regularly check and adjust the seat as the child grows. Ensuring a secure and proper fit in the child safety seat is essential for maximizing protection and reducing the risk of injury in the event of a collision. According to the guidance system for placing and harnessing the child 200-1, the system comprises a processor 202 to detect a type of child safety seat using an image sensor 206 and sensors 204 to receive data of the child safety seat through the connector 210. The system may communicate with the child safety seat and the vehicle control system 205 through the communication module 220 via the connector 210.

In an embodiment, the processor 202 is configured to detect a make and a model of the child safety seat at step 262. It could be via a connector 210 which is one or more of a wired connection and a wireless connection. Once the user places a child in the child safety seat, the system will detect a weight and a height of the child at step 264 via sensors embedded in the child safety seat, in the vehicle, and a combination of both. In an embodiment, an image may be processed to find the height of the child. In an embodiment, the child safety seat may have patterns from which a height of the child may be determined while processing an image. In an embodiment, the height and weight data are used to recommend a suitable safety seat for the child if the current safety seat is outgrown or not suitable due to its expiration date on the materials or damage to the child safety seat. The system then proceeds to generate a digital model of placing the child and harnessing the child at step 266. The digital model may be displayed on the information system associated with the vehicle and mirrored onto the user personal device. The system then proceeds to display step-by-step instructions for placing and harnessing the child at step 268. In this step, the digital model may present accurate positioning of each of the parts of the child in appropriate places. For example, head, torso, right leg, left leg, etc. Once the child is placed in the right manner, the system will then proceed to the next step. The system will provide time for the user to follow the step-by-step instructions for placing and harnessing the child at step 270. The system may guide the user on harness strapping the child securely, buckling the child in, placing of chest clip, etc. Each step is displayed on the infotainment system for the user to follow. The system further checks the installation at step 272. Once the user completes a step, the system will check and provide an indication via a signal on whether the step was executed correctly or not. In an embodiment the system may further provide a second signal after completing all steps accurately. The indication or signal may be via an audio, video, a tactile cue, and a combination thereof.

According to an embodiment of the system, the processor is operable to adjust a presentation of the step-by-step instructions and diagrams based on the proficiency level of the user, wherein the proficiency level is measured based on a time taken and an accuracy of the placing and harnessing of the child in the child safety seat after each step. According to an embodiment of the system, the system further comprises a memory module operatively connected to the processor, wherein the memory module stores procedures and protocols, a plurality of placing and harnessing of the child in the child safety seat data, and wherein the processor selects and displays an appropriate procedure and protocol based on the make and the model of the child safety seat.

According to an embodiment of the system, the system checks for a flat level and alignment of the child safety seat after the child is seated. According to an embodiment of the system, the system further checks that the belt of the child safety seat is installed straight and aligned accurately. The system is further configured to check a second parameter for a payout of a belt of the child safety seat, wherein the second parameter comprises a length of the belt of the child safety seat that is released for use before locking into place.

According to an embodiment of the system, the system recommends the placing and harnessing of the child in the child safety seat based on a manufacturer's manual. According to an embodiment of the system, the manufacturer's manual is accessed via a local memory or via a network.

According to an embodiment of the system, the processor is operable to display the digital model as a 3D model of the child safety seat to assist the user in performing the placing and harnessing of the child in the child safety seat, wherein the processor is operable to provide reminders and alerts to the user to ensure compliance with requirements of the placing and harnessing of the child in the child safety seat. According to an embodiment of the system, the processor is operable to display visual aids and diagrams to educate the user about the child safety seat that is appropriate for the child.

According to an embodiment of the system, the processor is operable to provide an interactive interface for the user to enter personal details comprising a name and age of the child. According to an embodiment of the system, the processor is operable to present virtual reality simulations of the placing and harnessing of the child in the child safety seat.

FIG. 2G shows a guidance system for adjusting a part of the child safety seat 200-2 according to a child according to an embodiment. According to the guidance system for adjusting a part of the child safety seat according to a child 200-2, the system comprises a processor 202 to detect a type of child safety seat using an image sensor 206 and the sensors 204 to receive data of the child safety seat through the connector 210. The system may communicate with the child safety seat and the vehicle control system 205 through the communication module 220 via the connector 210.

In an embodiment, the processor 202 is configured to detect a make and a model of the child safety seat at step 282. It could be via a connector 210 which is one or more of a wired connection and a wireless connection. Once the user places a child in the child safety seat, the system will detect a weight and a height of the child at step 284 via sensors embedded in the child safety seat, in the vehicle, and a combination of both. In an embodiment, an image may be processed to find the height of the child. In an embodiment, the child safety seat may have patterns from which a height of the child may be determined while processing an image. The system then proceeds to generate a digital model of the child safety seat along with adjustable parts associated with the child safety seat at step 286. The digital model may be displayed on the information system associated with the vehicle and mirrored onto the user personal device. The system then proceeds to determine the adjustable parts of the child safety seat according to the height and weight of the child at step 288. Most child safety seats have adjustable features that can be modified based on the child's weight and height. These include the harness straps, which can be moved up or down to fit the child's shoulders properly. The position of the harness buckle can also be adjusted to align with the child's lap. Many seats have adjustable recline angles to provide comfort and support at different stages of development. The adjustable headrest or head support ensures proper head and neck support as the child grows. Some seats offer adjustable seat heights to accommodate leg length. The system provides instructions for specific guidance based on the manufacturer's manual for adjusting these features for the child's safety and comfort. In this step, the digital model may present accurate positioning of each of the adjustable parts of the child safety seat. The system then displays step-by-step instructions for adjusting the adjustable parts of the child safety seat at step 290. The system will provide time for the user to follow the step-by-step instructions for adjusting the adjustable parts of the child safety seat at step 292. In an embodiment, the system will automatically adjust the adjustable parts of the child safety seat. The system may guide on child safety seats adjustable features, like harness straps, buckle position, recline angle, headrest position, and seat height, to accommodate the child's weight and height. These adjustments ensure a secure and comfortable fit for the child at different stages of growth. The system further checks the installation at step 294. Once the user completes a step the system will check it and provide an indication via a signal on whether the step was executed correctly or not. In an embodiment the system may further provide a second signal after completing all steps accurately. The indication or signal may be via one of an audio, a video, a tactile cue, and a combination thereof.

According to an embodiment of the system, the adjustable part comprises one or more of a head rest, an angle for the child safety seat, a cross band for the child safety seat, and the routing of the belt of the child safety seat. According to an embodiment of the system, the system is further operable to check and adjust a distance of the child safety seat from an object in front of the child safety seat for leg room of the child. According to an embodiment of the system, the object in front of the child safety seat comprises an instrument panel. According to an embodiment of the system, the object in front of the child safety seat comprises a front seat of the vehicle.

It is important to note that the flowchart or the method discussed herein provides a general overview of the steps involved in a computer vision system for child safety seat detection and implementation of guidance and checklists for the child safety seat installation and harnessing the child. The specific implementation may vary depending on the chosen algorithms, hardware setup, and vehicle manufacturer's design.

According to an embodiment of the system, the processor is operable to provide reminders and alerts to the user to ensure compliance with requirements of the installation. According to an embodiment of the system, the processor is operable to display visual aids and diagrams to educate the user about the child safety seat and recommend a new child safety seat based on a height and a weight of the child. According to an embodiment of the system, the processor is operable to provide an interactive interface to the user to enter personal details comprising a name and age of a child. According to an embodiment of the system, the processor is operable to present virtual reality simulations of the installation of the child safety seat. According to an embodiment of the system, the processor is operable to monitor a comfort of the child in the child safety seat, wherein the comfort monitoring is via one or more of a first movement of the child, a second movement of a child in the child safety seat due to driving, and a face expression of the child.

FIG. 2H shows a block diagram for a method depicting the steps involved in child size assessment in a child seat according to an embodiment. According to an embodiment, it is a method comprising, sensing, at least a portion of an interior of a vehicle, via a sensor module, wherein the sensor module comprises a radar sensor and an image sensor at step 2012; acquiring, information of the interior of the vehicle, via the sensor module at step 2014; determining, using the information of the interior of the vehicle via a machine learning module, a first characteristic of a child safety seat positioned in the interior of the vehicle at step 2016; retrieving, using the first characteristic of the child safety seat positioned in the interior of the vehicle, a specification information of the child safety seat at step 2018; determining, using the information of the interior of the vehicle via the machine learning module, a second characteristic of a child positioned in the child safety seat at step 2020; comparing, the second characteristic of the child with the specification information of the child safety seat at step 2022; and recommending, a suitability of the child safety seat at step 2024.

According to an embodiment of the method, the first characteristic of the child safety seat comprises one or more of the make, the model, the shape, and the size of the child safety seat. According to an embodiment of the method, the first characteristic is utilized to retrieve the specification information from data stored in a database stored in a local memory. According to an embodiment of the method, the first characteristic is utilized to retrieve the specification information from data stored in a database stored in a cloud memory. According to an embodiment of the method, the method is further operable for connecting the vehicle and the child safety seat, via a communication module. According to an embodiment of the method, the first characteristic is received via the communication module.

According to an embodiment of the method, the method is further operable to display a message, wherein the message comprises a position of the child safety seat, a direction of the child safety seat, and the suitability of the child safety seat.

According to an embodiment of the method, the method is further operable to recommend a position for the child safety seat and a direction of installation of the child safety seat.

According to an embodiment of the method, the suitability of the child safety seat is determined by comparing a height of the child, a weight of the child, an age of the child, to a height limit, a weight limit, an age limit of the specification information of the child safety seat.

According to an embodiment of the method, the suitability of the child safety seat is determined by comparing a behavior of the child to a height limit, a weight limit, an age limit of the specification information of the child safety seat.

According to an embodiment of the method, the behavior of the child comprises movement of the child in the child safety seat and a pressure created on a portion of a belt of the child safety seat. According to an embodiment of the method, the behavior of the child comprises an interaction of the child with the child safety seat.

FIG. 2I shows a block diagram showing a child size assessment system 2040 according to an embodiment. According to an embodiment, it is a system comprising a sensor module 2046 comprising a radar sensor 2048 and an image sensor 2050; a processor 2042; and a memory 2044 communicably coupled to the processor comprising computer-readable instructions that when executed by the processor cause the processor to: sense, at least a portion of an interior of a vehicle, via the sensor module at step 2012; acquire, information of the interior of the vehicle, via the sensor module at step 2014; determine, using the information of the interior of the vehicle via a machine learning module, a first characteristic of a child safety seat positioned in the interior of the vehicle at step 2016; retrieve, using the first characteristic of the child safety seat positioned in the interior of the vehicle, a specification information of the child safety seat at step 2018; determine, using the information of the interior of the vehicle via the machine learning module, a second characteristic of a child positioned in the child safety seat at step 2020; compare, the second characteristic of the child with the specification information of the child safety seat at step 2022; and recommend, a suitability of the child safety seat at step 2024.

According to an embodiment of the system, the first characteristic of the child safety seat comprises one or more of the make, the model, the shape, and the size of the child safety seat. According to an embodiment of the system, the first characteristic is utilized to retrieve the specification information from data stored in a database stored in a local memory. According to an embodiment of the system, the first characteristic is utilized to retrieve the specification information from data stored in a database stored in a cloud memory. According to an embodiment of the system, the system further comprises a communication module connecting the vehicle and the child safety seat. According to an embodiment of the system, the first characteristic is received via the communication module.

According to an embodiment of the system, the second characteristic of the child comprises one or more of the height of the child, the weight of the child, an age of the child, a behavior of the child, and the fit of the child in the child safety seat.

According to an embodiment of the system, determining the suitability comprises information that indicates at least one of continuing use of the child safety seat, a replacement for the child safety seat, and an upgradation of the child safety seat. According to an embodiment of the system, the suitability of the child safety seat is determined by comparing a height of the child, a weight of the child, an age of the child, to a height limit, a weight limit, an age limit of the specification information of the child safety seat. According to an embodiment of the system, the suitability of the child safety seat is determined by comparing a behavior of the child to a height limit, a weight limit, an age limit of the specification information of the child safety seat. According to an embodiment of the system, the behavior of the child comprises movement of the child in the child safety seat and a pressure created on a portion of a belt of the child safety seat. According to an embodiment of the system, the behavior of the child comprises an interaction of the child with the child safety seat.

FIG. 2J shows a block diagram for a non-transitory computer medium for storing the steps involved in child size assessment in a child seat according to an embodiment. According to an embodiment, it is a non-transitory computer-readable medium 2074 having stored thereon instructions executable by a computer system 2070 to perform operations comprising, sensing, at least a portion of an interior of a vehicle, via a sensor module, wherein the sensor module comprises a radar sensor and an image sensor at step 2012; acquiring, information of the interior of the vehicle, via the sensor module at step 2014; determining, using the information of the interior of the vehicle via a machine learning module, a first characteristic of a child safety seat positioned in the interior of the vehicle at step 2016; retrieving, using the first characteristic of the child safety seat positioned in the interior of the vehicle, a specification information of the child safety seat at step 2018; determining, using the information of the interior of the vehicle via the machine learning module, a second characteristic of a child positioned in the child safety seat at step 2020; comparing, the second characteristic of the child with the specification information of the child safety seat at step 2022; and recommending, a suitability of the child safety seat at step 2024. A software application 2076 may be stored on a non-transitory computer-readable medium 2074 and executed with the processor 2072 of the computer system 2070.

According to an embodiment of the non-transitory computer-readable medium, the image sensor comprises a camera comprising a computer vision module. According to an embodiment of the non-transitory computer-readable medium, the camera is operable for capturing a video in real-time and enabling continuous monitoring.

According to an embodiment of the non-transitory computer-readable medium, the video is analyzed for detecting a behavior of the child, wherein the behavior comprises one or more of an attempt to unbuckle a harness of the child safety seat, a restlessness of the child seated in the child safety seat, an attempt to exit the child safety seat, and the legroom available for the child in the child safety seat. According to an embodiment of non-transitory computer-readable medium, the sensor module is further operable to detect one or more of an expiration date of the child safety seat, a crash history of the child safety seat, and a recall notification for the child safety seat.

FIG. 2K shows a block diagram showing the steps involved in a child seat suitability assessment by the vehicle according to an embodiment. It comprises the steps wherein the vehicle identifies a child seat installed in the vehicle at step 2092; the vehicle establishes seat usage limitations at step 2094; the vehicle assesses the size and age of the child 2096; and the vehicle assesses if the child seat is suitable for the child at step 2098.

Vehicle identifies a child seat installed in the vehicle at step 2092 by using a camera and/or a radar. In an embodiment, the radar may be a millimeter-wave (mmWave) Radar. It may further use more than one sensor at a time to identify the child seat that is installed. Once the child seat is identified, then the vehicle identifies the make and model of the child seat and the presence of the child. The vehicle further proceeds to identify the essential characteristics of the child seat.

Essential characteristics comprise analyzing: whether the child safety seat is appropriately sized and fitted to match the child's age, weight, and height, ensuring a snug and secure fit; determining age and weight limits and guiding caregivers on whether the child safety seat is suitable at the current child's developmental stage and size; determining that the child safety seat use is aligned with the recommended direction of use, whether rear-facing, forward-facing, or booster, based on the child's age and size; determining whether the child safety seat is meeting established safety standards, such as Federal Motor Vehicle Safety Standards (FMVSS) in the United States or Economic Commission for Europe (ECE) developed regulations, ECE R44/04 or ECE R129 (i-Size) in Europe; determining correctness of installation, correctness in securing and adjusting the harness system, installation of side-impact protection features, and correct adjustment of recline positions for safety and comfort; determining whether the child safety seat is clean and hygienic; and determining expiration dates.

Vehicle establishes seat usage limitation at step 2094 where the vehicle fetches, from the model database information, the child seat usage. The model database could be either stored locally or in a cloud database. The model database may comprise all the details related to the make and model of the child safety seat. It can be accessed by using the model identification (id) or the make and model information. Further, the database may comprise data related to different types of child safety seats segregated according to make and model. In an embodiment, the data for child seat usage limits can be accessed via a communication module connecting the child safety seat and the vehicle. Then, from the child seat characteristics, the generic usage limits are defined.

Vehicle assesses the size and age of the child 2096 by using camera, radar, or any other means. Other means could comprise user input data via vehicle infotainment system or a user device. Other means may comprise accessing child data from a user's personal device. A trained machine learning model for child identification and assessment extracts critical features of the child's posture, stature, and size from the input taken from the camera and/or radar.

Vehicle assesses if the child seat is suitable for the child at step 2098 by comparing the child stature with the child seat usage limits. The vehicle then informs the driver of the vehicle of non-optimal seat usage for the child including whether to use or not to use the child seat.

FIG. 3 shows a child safety seat/child restraint system with various sensors according to an embodiment. The child restraint system 300 may be a restraint system equipped with safety straps to hold a child in a seated position in a vehicle 380. As set out in more detail below, the child restraint system 300 may include sensors that may respond to relative conditions between a child, child restraint system 300, and a vehicle by providing human-perceptible signals in response to the attainment of predetermined conditions. Vehicle 380 may include a lap/shoulder belt 382 extending from a back seat retractor 384 and having a lap/shoulder belt buckle tongue 386 configured to mate to a vehicle seat belt buckle 388 secured to vehicle 380. Vehicle 380 additionally may include a latch anchor 390. Latch anchor 390 may have a metal plate 392 attached to vehicle 380 and latch anchor ring 394 attached to metal plate 392. Generally, lap/shoulder belt 382 and latch anchor 390 may aid in securing the child restraint system 300 to vehicle 380.

Child restraint system 300 may include seat 302. Seat 302 may have components attached to form an area contoured to support a body by the back, buttocks, and thighs and provide resting areas for the arms of the body. For example, seat 302 may include a head support 304, a back support 306, a torso support 308, a right leg support 310, a left leg support 312, a right arm support 314, and a left arm support 316. Each may function as their name describes. For example, head support 304 may support a child's head, back support 306 may support a child's back, and left leg support 312 may support a child's left leg. According to an embodiment of the system, the system further checks for heavy wear (e.g., coats) on the child to ensure that the belt of the child safety seat is properly securing the child.

Seat 302 additionally may include right slots 318, a right harness strap 320, left slots 322, a left harness strap 324, a crotch strap 326, a child seat buckle 328, a chest clip 330, a tether strap 332 having a hook 334, and a locking clip 336. Right slots 318 may provide usage to adjust a length of the right harness strap 320, and left slots 322 may provide usage to adjust a length of left harness strap 324. Right harness strap 320 and left harness strap 324, each, may be attached to seat 302 and be secured into the child seat buckle 328. Child seat buckle 328 may be attached to a crotch strap 326, which, in turn may be attached to seat 302. A chest clip 330 may removably secure right harness strap 320 and left harness strap 324 to each other. Tether strap 332 may be attached near head support 304, and hook 334 of tether strap 332 may be attached to latch anchor ring 394 to secure child restraint system 300 to vehicle 380.

Child restraint system 300 additionally may include base 338. Base 338 may be a pedestal upon which seat 302 may be secured. Base 338 may include a belt passageway 340 and a base hole 342. Lap/shoulder belt buckle tongue 386 and lap/shoulder belt 382 may be passed from back seat retractor 384 through belt passageway 340 and be secured to vehicle seat belt buckle 388 to secure child restraint system 300 to vehicle 380. In those situations, where lap/shoulder belt buckle tongue 386 slides freely along lap/shoulder belt 382 without locking into place, locking clip 336 may be threaded onto lap/shoulder belt 382 and slid to a position that is within about one-half an inch of vehicle seat belt buckle 388.

Child restraint system 300 may include one or more sensors to determine the presence of a child in seat 302, determine whether a child is sitting correctly in seat 302, determine whether any one of right harness strap 320, left harness strap 324, chest clip 330, tether strap 332, lap/shoulder belt 382, and locking clip 336 are properly secured. In particular, child restraint system 300 may include a back sensor 344 positioned in back support 306, a torso sensor 346 positioned in torso support 308, a right leg sensor 348 positioned in right leg support 310, and a left leg sensor 350 positioned in left leg support 312. Each sensor 344, 346, 348, and 350 may indicate the presence of an associated body part by detecting a force applied by that body part. For example, back support 306 may detect that a child is leaning against seat 302 and right leg sensor 348 may detect a weight of a child's right leg as that leg dangles over right leg support 310.

In addition to sensors 344, 346, 348, and 350, child restraint system 300 may include a child seat buckle detector 352, a chest clip detector 354, a vehicle seat belt buckle detector 356, a locking clip detector 358, a latch anchor detector 360, an acceleration detector 362, first movement detector 364, a second movement detector 365, a controller 366, a speaker 368, a power source 370, and a data input/output port 372. Importantly, sensors 344, 346, 348, and 350, detectors 352, 354, 356, 358, 360, 362, 364, 365, controller 366, speaker 368, and power source 370 all may be contained within child restraint system 300. In an embodiment, some of the systems and sensors may be installed in the vehicle seat instead of child seat.

Both right buckle tongue and left buckle tongue may be configured to fit within the child seat buckle and be secured in a position that may be fixed relative to the buckle tongue detector. When the right buckle tongue and left buckle tongue are in this fixed position, the child seat buckle may be deemed to be properly secured. Both right buckle tongue and left buckle tongue may include material arranged to set up a magnetic field between right buckle tongue and left buckle tongue when these two elements are within a predetermined distance from each other. Buckle tongue detectors may be positioned in this magnetic field when the right buckle tongue and left buckle tongue are fixed in position relative to the buckle tongue detector. If either the right buckle tongue or left buckle tongue are moved relative to one another, that may indicate that the child seat buckle is not properly secured. If either the right buckle tongue or left buckle tongue are moved relative to one another, the magnetic field may change, and that change may be picked up by the buckle tongue detector. As a result of this change, buckle tongue detectors may generate a buckle tongue signal and send buckle tongue signals along wires. A Buckle tongue signal may be a first buckle tongue signal to signal a modification to an existing magnetic field and may be a second buckle tongue signal to signal a non-existing magnetic field. To signal a modification to an existing magnetic field, at least one of right buckle tongue and left buckle tongue should be properly secured to child seat buckle and the remaining buckle tongue should be close enough to the properly secured buckle tongue to generate a magnetic field between right buckle tongue and left buckle tongue.

Similarly, chest clip detectors may have a configuration similar to child seat buckle detectors. Opposing chest clip buckle tongues may include material arranged to set up a magnetic field between the opposing chest clip buckle tongues when these two elements are within a predetermined distance from each other. A chest clip buckle detector may be positioned in the magnetic field to pick up any relative movement between the opposing chest clip buckle tongues. Relative movement between the opposing chest clip buckle tongues may indicate that the chest clip may not be properly installed, and a signal may be generated and sent to the controller. Similar arrangements can be performed using sensors for every check that is being made.

According to an embodiment of the system, the system further checks for a support for the head of the child to ensure that the head of the child is well supported and is accurately placed.

In an embodiment, a computer vision system is used to detect various aspects of the installation. Vehicle interior sensing capabilities may include interior cameras and sensors that can be used to detect the presence of accessories such as child safety seats, passengers, to monitor their posture and movements, and to adjust the seat belts, airbags, and other safety systems accordingly. Advanced biometric sensors can identify individual passengers and customize the vehicle settings to their preferences, such as seat position. Advanced sensors and cameras can detect the presence of occupants in different seating positions within the vehicle. This information may be used to optimize safety features such as seat belt tension and adaptive restraints. Interior cameras are small cameras which may be strategically positioned within the vehicle to capture visual information. They may be used for various purposes, including child safety seat detection, child detection, occupant detection, gesture recognition, facial recognition, and monitoring the overall interior space. The cameras may utilize different technologies such as RGB (color) cameras, infrared cameras for night vision, or depth-sensing cameras for enhanced spatial understanding. Further, interior sensors are designed to detect and measure different parameters within the vehicle's interior. They can include pressure sensors, motion sensors, ambient light sensors, temperature sensors, humidity sensors, and air quality sensors, among others. These sensors provide data on occupant presence, seating positions, environmental conditions, and other relevant factors. The information captured by interior cameras and sensors is utilized by the vehicle's onboard systems and algorithms to enable child safety seat detection and to analyze the steps of installation and whether the user has installed the child safety seat correctly or not. For instance, the data from interior cameras can be processed to detect the seat belt and check whether the seat belt is passing through designated locations or not. It may check using a computer vision system whether the seat belt is crooked or not. Vehicle's interior sensing system can provide feedback or alerts to the driver if it detects any issues with the child seat installation.

Interior cameras can be used to detect the shape and size of a child seat and determine whether it is properly installed. They can recognize specific patterns or features associated with child seats and compare them to a database of known child seat models and installation guidelines. This allows the system to determine if the child seat is correctly positioned, secured, and adjusted according to recommended guidelines. According to an embodiment of the system, the processor is operable to capture, via a camera, an interior of the vehicle.

According to an embodiment of the system, the system comprises a computer vision module comprising a shape recognizing camera. According to an embodiment of the system, the processor is operable to capture, via a camera, an interior of the vehicle. According to an embodiment of the system, the information system comprises an infotainment system of the vehicle. According to an embodiment of the system, the system comprises a shape recognizing camera, wherein the shape recognizing camera comprises an image processing algorithm to identify and classify objects based on their shapes. According to an embodiment of the system, the child safety seat comprises weight sensors to determine the weight of the child. According to an embodiment of the system, the processor is operable to detect a position of a harness chest clip and to guide for the optimal adjustment for accurate positioning.

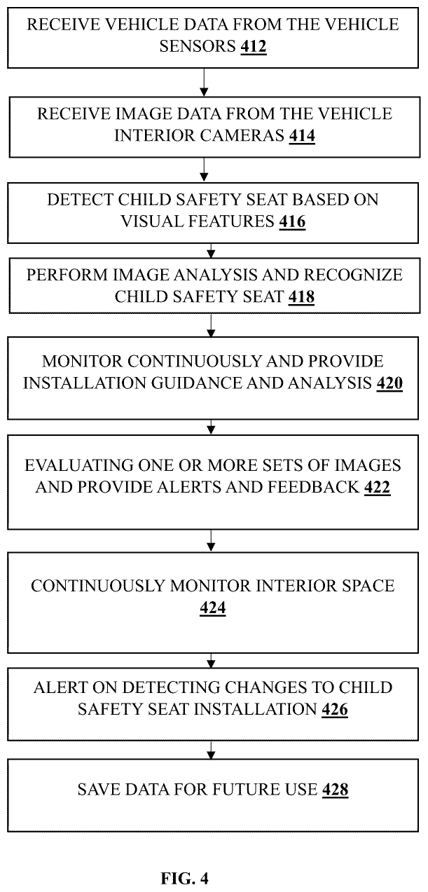

FIG. 4 shows a block diagram for a method depicting the steps involved in a computer vision system for detecting child safety seat installation. It comprises receiving vehicle data from the vehicle sensors at step 412. The system may receive image data from vehicle interior cameras at step 414. The input from interior cameras may be images or video footage of the vehicle's interior. The interior cameras may be installed at strategic locations. For example, in seats directly in front of the back seat where a child seat may be installed or over the roof capturing the top view. In an embodiment, data from one or more cameras may be combined. The system then performs image analysis and detects a child safety seat based on visual features at step 416. The system then performs image analysis and recognizes or identifies the child safety seat at step 418. In an embodiment, the images are analyzed for capturing a child seat company name, tags, size, shape, or any other instructions. The captured images or video frames are preprocessed to enhance image quality and reduce noise. This may involve operations such as resizing, noise reduction, and color normalization, etc. An object detection algorithm may be used for detecting various objects and various aspects of the objects. In an embodiment, a bounding box method may be used to extract various safety features. Computer vision algorithms are applied to detect objects within images. Specifically, the system focuses on identifying child safety seats based on their visual features, such as shape, size, and color. Object detection techniques like Haar cascades, convolutional neural networks (CNN), or other machine learning models can be used for this task. In an embodiment, child seat recognition algorithms are executed. Once a potential child safety seat is detected, the system compares its visual characteristics with a database of known child seat models and installation guidelines. This comparison helps determine if the detected object matches a valid child safety seat.

Convolutional Neural Networks (CNNs) may be used in object detection tasks due to their ability to analyze visual data effectively. In the context of object detection, CNNs are trained using labeled datasets consisting of images and corresponding bounding box annotations. The CNN architecture includes layers for feature extraction, region proposal, and classification/localization. Initially, CNN extracts high-level features from input images, gradually learning more complex patterns. The region proposal network generates potential object bounding box proposals, and CNN classifies the proposed regions and refines the bounding box coordinates. Non-maximum suppression is applied to remove redundant detections. During inference, the trained CNN model processes new images to detect and localize objects, providing object labels and bounding box coordinates. CNNs enable accurate object detection by learning discriminative features and patterns, enhancing the performance of systems designed for object recognition and localization. A CNN architecture suitable for object detection is chosen. Popular architectures for object detection include Faster Region-Based Convolutional Neural Network (R-CNN), YOLO (You Only Look Once), and SSD (Single Shot MultiBox Detector). These architectures typically consist of convolutional layers for feature extraction, followed by region proposal or detection layers.

The system may monitor continuously and provide installation guidance and analysis at step 420. For this the system may provide a 3D model of the child car seat on the infotainment system. For installation analysis, the system may analyze the position and alignment of the detected child seat. It examines factors such as seat orientation, angle, and attachment points to assess if the seat is installed properly via computer vision algorithms and sensor data. Sensors may be located at various places including attachment points to provide installation checks. The system may then proceed to evaluate one or more sets of images and provide alerts and feedback at step 422. Based on the analysis of sensor data and image data, the system provides feedback or alerts to the driver or occupants. This feedback can be in the form of visual indicators on the vehicle's dashboard display or audible warnings to ensure that any issues with the child seat installation are addressed.

According to an embodiment of the system, the processor is operable to monitor a comfort of the child in the child safety seat, wherein the comfort monitoring comprises one or more of a first movement of the child, a second movement of the child in the child safety seat due to driving, and a face expression of the child. According to an embodiment of the system, the system is operable to capture, via the second sensor, an interior of the vehicle, wherein the second sensor comprises a camera.

The computer vision system continuously monitors the interior scene to detect any changes in the child seat position or installation status at step 424. It updates the analysis in real-time and provides ongoing feedback as necessary. The system may provide alerts upon detecting changes to child safety seat installation at step 426. The data collected during installation and after installation when the vehicle is in motion is stored or saved for future use at step 428. The data may be stored locally or on the cloud network.

According to an embodiment of the system, the child safety seat is operable to collect real-time data about a child using the child safety seat, wherein the real-time data comprises one or more of a height, a weight, a movement, a health parameter, and a face detection. According to an embodiment of the system, the processor is operable to display real-time data from the child safety seat on the information system. According to an embodiment of the system, the processor is operable to detect an anomaly based on real-time data collected during the installation of the child safety seat. According to an embodiment of the system, the processor is operable to provide personalized recommendations to the user via the information system based on a history of the installation of the child safety seat and from results of real-time data collected by the child safety seat. According to an embodiment of the system, the processor is operable to provide decision support to the user by analyzing the real-time data of the child and recommending a new child safety seat according to the weight and the height of the child. According to an embodiment of the system, the real-time data collected from the child safety seat via the sensor is transferred via one or more of a connector, near field communication, and Bluetooth®.

According to an embodiment of the system, the child safety seat is operable to collect real-time data about the child using the child safety seat, wherein the real-time data comprises one or more of a height, a weight, a movement, a health parameter, and a face detection.

According to an embodiment of the system, the processor is operable to display real-time data from the child safety seat on the information system. According to an embodiment of the system, the processor is operable to detect an anomaly based on real-time data collected during the placing and harnessing of the child in the child safety seat. According to an embodiment of the system, the processor is operable to provide personalized recommendations to the user via the information system based on a history of the placing and harnessing of the child in the child safety seat and from the real-time data collected by the child safety seat. According to an embodiment of the system, data collected from the child safety seat via the first sensor and the second sensor is transferred via one or more of a connector, a near field communication, and Bluetooth®.

According to an embodiment of the system, the system further comprises a data communication module operatively connected to the processor, wherein the data communication module is operable to transmit the real-time data collected during the installation of the child safety seat to a remote server for further analysis. According to an embodiment of the system, the information system is further operable to present warnings and alerts to the user based on real-time data collected by the child safety seat.

In an embodiment, the system may be designed to reset or re-evaluate the child seat detection process periodically or when triggered by specific events, such as a change in vehicle dynamics or system initialization.

According to an embodiment, it is a system comprising: an information system of a vehicle operable to present step-by-step instructions to guide a user for installation of a child safety seat; and a processor operatively connected to the information system operable to control a display of the step-by-step instructions; and wherein the processor is operable to, recognize, via a sensor, a make, and a model of the child safety seat; generate a digital model of the child safety seat on the information system of the vehicle; and display the step-by-step instructions for the installation of the child safety seat.

According to an embodiment, it is a method comprising: recognizing, via a sensor, a make and a model of a child safety seat that is being installed in a vehicle; generating, via a processor, a digital model of the child safety seat on an information system of the vehicle; and displaying step-by-step instructions for an installation of the child safety seat on the display of the information system of the vehicle for a user to follow.

According to an embodiment of the method, the method is operable for providing time for the user to follow each step of the step-by-step instructions; checking after each step of the installation of the child safety seat for accuracy of completion of each step; continue displaying a next step after completing a previous step; providing a first signal after completion of each step of the installation of the child safety seat; and generating a second signal after completing all the steps of the installation.

According to an embodiment of the method, the step-by-step instructions for the installation of the child safety seat comprises: guiding a direction of a placement of the child safety seat; checking and confirming the direction of the placement of the child safety seat; guiding a connection of the child safety seat with an attachment point; checking and confirming that the child safety seat is accurately connected with the attachment point; guiding a routing of a vehicle seat belt to pass through one or more points on the child safety seat; checking and confirming that the routing of the vehicle seat belt is accurate and is passing through the one or more points on the child safety seat; guiding the user for a tautness on the vehicle seat belt; and checking and confirming the tautness of the vehicle seat belt; and guiding the user for a distance between the child safety seat and an object in front of the child safety seat; and checking and confirming the distance between the child safety seat and the object in front of the child safety seat.

According to an embodiment it is a system comprising: an information system of a vehicle operable to present step-by-step instructions to guide a user for installation of a child safety seat; and a processor operatively connected to the information system operable to control a display of the step-by-step instructions; and wherein the processor is operable to, recognize, via a sensor, a make, and a model of the child safety seat; generate, via the processor, a digital model of the child safety seat on the information system of the vehicle; display the step-by-step instructions for the installation of the child safety seat; and automatically execute by a control system, the step-by-step instructions for the installation of the child safety seat.

According to an embodiment of the system, the processor is operable to automatically execute each step of the step-by-step instructions; check the installation after each step of the child safety seat instructions for accuracy of completion of each step; provide a first signal after completion of each step of the installation of the child safety seat accurately; and generate a second signal after accurately completing all the steps of the installation.

According to an embodiment of the system, the step-by-step instructions for the installation of the child safety seat comprises: automatically place the child safety seat in a direction; automatically connect the child safety seat to an attachment point; automatically route a vehicle seat belt to pass through one or more points on the child safety seat; automatically adjust the vehicle seat belt for tautness; and automatically adjust for a distance between the child safety seat and an object in front of the child safety seat.

According to an embodiment of the system, the processor is operable to: check and confirm the direction of a placement of the child safety seat; check and confirm that the child safety seat is accurately connected to the attachment point/s; check and confirm that the vehicle seat belt is passing accurately through the one or more points on the child safety seat; check and confirm for the tautness on the vehicle seat belt; and check and confirm the distance between the child safety seat and the object in front of the child safety seat.

According to an embodiment it is a system comprising: an information system of a vehicle operable to present step-by-step instructions to guide a user in placing a child in a child safety seat; and a processor operatively connected to the information system operable to control a display of the step-by-step instructions; and wherein the processor is operable to, recognize, via a first sensor, a make, and a model of the child safety seat; recognize, via a second sensor, a height of the child, and a weight of the child that is seated in the child safety seat; generate a digital model of the placing of the child in the child safety seat on the information system of the vehicle; display the step-by-step instructions for placing and harnessing of the child in the child safety seat; and wherein the processor is operable to determine an optimal adjustment of the child safety seat according to a first parameter of the child that is being placed in the child safety seat, wherein the first parameter is one or more of the height of the child and the weight of the child.

According to an embodiment of the system, the processor is operable to provide time for the user to follow each step of the step-by-step instructions; continue displaying a next step after completing a previous step; check at an end of each step for accuracy of completion of each step; provide a first signal after completion of each step; and provide a second signal after completing all the steps.

According to an embodiment of the system, the step-by-step instructions comprises: guiding the placing and harnessing of the child in the child safety seat; checking and confirming the placing and harnessing of the child in the child safety seat; guiding a routing of a belt of the vehicle around the child in the child safety seat; checking and confirming the belt of is routed accurately around the child; guiding for a tautness of the belt around the child in the child safety seat; checking and confirming for the tautness on the belt around the child; determining an adjustable part of the child safety seat; guiding to accurately adjust the adjustable part of the child safety seat according to the height of the child and the weight of the child; and checking and confirming that the adjustable part is accurately adjusted according to the height of the child and the weight of the child.

According to an embodiment of the system, the first sensor is further configured to recognize a size and a year of manufacture of the child safety seat. According to an embodiment of the system, the first sensor comprises one or more of a camera, a LiDAR, a radar, and a Radio Frequency Identification sensor configured to recognize the make and the model of the child safety seat. According to an embodiment of the system, the child safety seat comprises a plurality of sensors, wherein the plurality of sensors comprises a seat belt sensor, a position sensor, a pressure sensor, a liquid detection sensor, a weight sensor, an infrared sensor, an optical sensor, a comfort detection sensor, a moisture sensor, a temperature sensor, and an audio sensor.

According to an embodiment of the system, the vehicle comprises a plurality of sensors, wherein the plurality of sensors comprises a seat belt sensor, a position sensor, a pressure sensor, a liquid detection sensor, weight sensor, an infrared sensor, an optical sensor, a comfort detection sensor, a moisture sensor, a temperature sensor, and an audio sensor. According to an embodiment of the system, a belt of a seat in the vehicle comprises a plurality of sensors, wherein the plurality of sensors comprises a position sensor, a pressure sensor, a liquid detection sensor, a weight sensor, an infrared sensor, an optical sensor, a comfort detection sensor, a moisture sensor, a temperature sensor, and an audio sensor.

According to an embodiment of the system, a belt of the child safety seat comprises a plurality of sensors, wherein the plurality of sensors comprises a position sensor, a pressure sensor, a liquid detection sensor, a weight sensor, an infrared sensor, an optical sensor, a comfort detection sensor, a moisture sensor, a temperature sensor, and an audio sensor. According to an embodiment of the system, the first signal comprises one or more of a text message, a visual cue, a sound alert, a tactile cue, and a vibration. According to an embodiment of the system, the second signal comprises one or more of a text message, a visual cue, a sound alert, a tactile cue, and a vibration. According to an embodiment of the system, an ignition system of the vehicle is operable to start upon completing all the steps of placing and harnessing the child in the child safety seat accurately.

According to an embodiment, it is a method comprising: recognizing, via a first sensor, a make and a model of a child safety seat in a vehicle; recognizing, via a second sensor, a child that is seated in the child safety seat, a height of the child, and a weight of the child; generating, via a processor, a digital model of placing the child in the child safety seat on an information system of the vehicle; and displaying step-by-step instructions for placing and harnessing of the child in the child safety seat for a user; and wherein the method is operable to determine an optimal adjustment of the child safety seat according to the child that is being placed in the child safety seat.

According to an embodiment of the system, the digital model comprises a 3D image generated by capturing a plurality of 2D images of the child and the child safety seat from a plurality of angles. According to an embodiment of the system, the information system presents step-by-step instructions in a user-friendly format. According to an embodiment of the system, the processor is further operable to receive feedback from the user regarding the quality and effectiveness of the step-by-step instructions presented on the information system, and to update the step-by-step instructions based on the feedback received. According to an embodiment, it is a method for providing step-by-step instructions for the placing and harnessing of the child in the child safety seat.

According to an embodiment, it is a system comprising: an information system of a vehicle operable to present step-by-step instructions to guide a user in placing a child in a child safety seat; and a processor operatively connected to the information system operable to control a display of the step-by-step instructions; and wherein the processor is operable to, recognize, via a first sensor, a make, and a model of the child safety seat; recognize, via a second sensor, the child that is seated in the child safety seat, a height of the child, and a weight of the child; generate, via the processor, a digital model of the child sitting in the child safety seat on the information system of the vehicle; display and execute the step-by-step instructions for placing and harnessing of the child in the child safety seat; and automatically adjust by a control system a tautness of the belt of the child safety seat; and wherein the processor is operable to determine an optimal adjustment of the child safety seat according to the child that is being placed in the child safety seat.

According to an embodiment of the system, the processor is operable to automatically execute each step of the step-by-step instructions; check at an end of each step for accuracy of completion of each step; provide a first signal after completion of each step; and provide a second signal after completing all the steps.

According to an embodiment of the system, the step-by-step instructions comprises guiding the placing and harnessing of the child in the child safety seat; automatically routing of a belt of the child safety seat with the child in the child safety seat; automatically adjust for a tautness on the belt of the child safety seat around the child; determining an adjustable part of the child safety seat; and automatically adjust the adjustable part of the child safety seat.

According to an embodiment of the system, the processor is operable to: check and confirm the placing and harnessing of the child in the child safety seat; check and confirm the belt of the child safety seat is routed accurately around the child; check and confirm for the tautness on the belt of the child safety seat around the child; and check and confirm that the adjustable part is accurately adjusted according to the height of the child and the weight of the child.

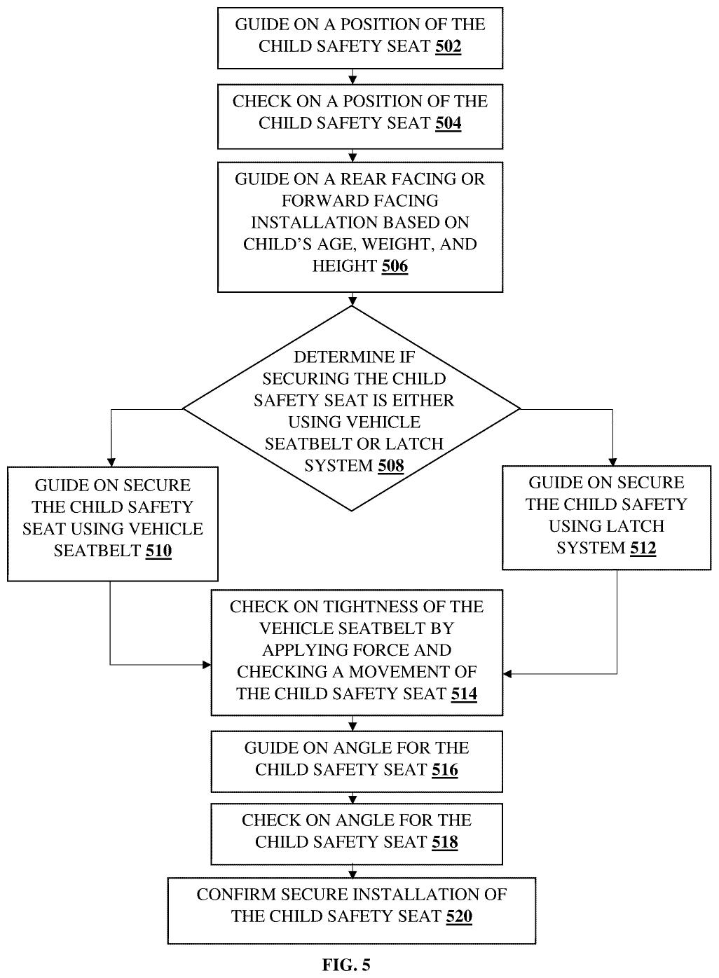

FIG. 5 shows a checklist of a child safety seat installation according to an embodiment. The system ensures that each step of the installation is checked and ensures that the step is followed correctly, and the installation is proper. According to an embodiment, the system is configured to guide a user on a position of the child safety seat at step 502, check on a position of the child safety seat at step 504, guide on a rear facing or forward facing installation based on child's age, weight, and height at step 506. The system determines if securing the child safety seat is either using a vehicle seat belt or latch system at step 508. It may then guide the user on securing the child safety seat using vehicle seat belt at step 510 if the seat is to be secured by the vehicle seat belt. If it is determined that securing the child safety seat is by using the latch system, then the system may guide on securing the child safety using the latch system at step 512. The system is then configured to check the tautness of the vehicle seat belt by applying force and checking the movement of the child safety seat at step 514, guide the user on the correct angle for the child safety seat at step 516. Check on the angle of the child safety seat at step 518 and confirm secure installation of the child safety seat at step 520. The system and the method are enabled to produce a first signal for confirming the proper execution of the step. If the user needs further guidance, the system will provide additional information to help the user carry out the step successfully. According to an embodiment of the system, the system further checks that the child safety seat is installed straight and aligned accurately with a vehicle seat.

According to an embodiment of the system, the system is further configured to check a parameter for a payout of a belt, wherein the parameter comprises a length of the belt that is released for use before locking a further release. According to an embodiment of the system, the system further checks for a front brace to stand on a vehicle seat accurately.

According to an embodiment of the system, the first signal comprises one or more of a text message, a visual cue, a sound alert, a tactile cue, and a vibration; and wherein the second signal comprises one or more of a text message, a visual cue, a sound alert, a tactile cue, and a vibration. According to an embodiment of the system, the sensor is further configured to recognize a size and a year of manufacture of the child safety seat. According to an embodiment of the system, an ignition system of the vehicle is coupled with the second signal and is operable upon completing all the steps of the installation accurately.

The system ensures that each step of placing the child and harnessing the child is checked and that each step is followed correctly, and the placement of the child is proper. Further the system determines if there are any adjustable parts in the child safety seat according to the height and weight of the child. According to an embodiment, the system is configured to guide on placing the child in the child safety seat, check on a position of the child in the child safety seat, check child's age, weight, and height, guide on routing of the belt of the child safety seat, check routing of the belt of the child safety seat, guide on buckling the belt of child safety seat, check on buckling the belt of child safety seat, guide on positioning chest clip of the belt of child safety seat, check on positioning chest clip of the belt of child safety seat, guide on angle for the child safety seat, check on angle for the child safety seat, guide on adjusting the adjustable parts of the child safety seat, check on adjusting the adjustable parts of the child safety seat, confirm secure harnessing of the child in the child safety seat. The system and the method are enabled to produce a first signal for confirming the proper execution of the step. If the user needs further guidance, the system will provide additional information to help the user carry out the step successfully.

According to an embodiment of the system, the processor is operable to: provide time for the user to follow each step of the step-by-step instructions; check the installation of the child safety seat after each step for accuracy of completion of each step; continue displaying a next step after completing a previous step; provide a first signal after completion of each step of the installation of the child safety seat accurately; and generate a second signal after completing all the steps of the installation accurately.

According to an embodiment of the system, the step-by-step instructions for the installation of the child safety seat comprises: guiding a direction of a placement of the child safety seat; checking and confirming the direction of the placement of the child safety seat; guiding a connection of the child safety seat with an attachment point; checking and confirming that the child safety seat is accurately connected with the attachment point; guiding a routing of a vehicle seat belt to pass through one or more points on the child safety seat; checking and confirming the routing of the vehicle seat belt is accurate and is passing through the one or more points on the child safety seat; guiding the user for a tautness on the vehicle seat belt; and checking and confirming for the tautness on the vehicle seat belt; and guiding the user for a distance between the child safety seat and an object in front of the child safety seat; and checking and confirming the distance between the child safety seat and the object in front of the child safety seat.

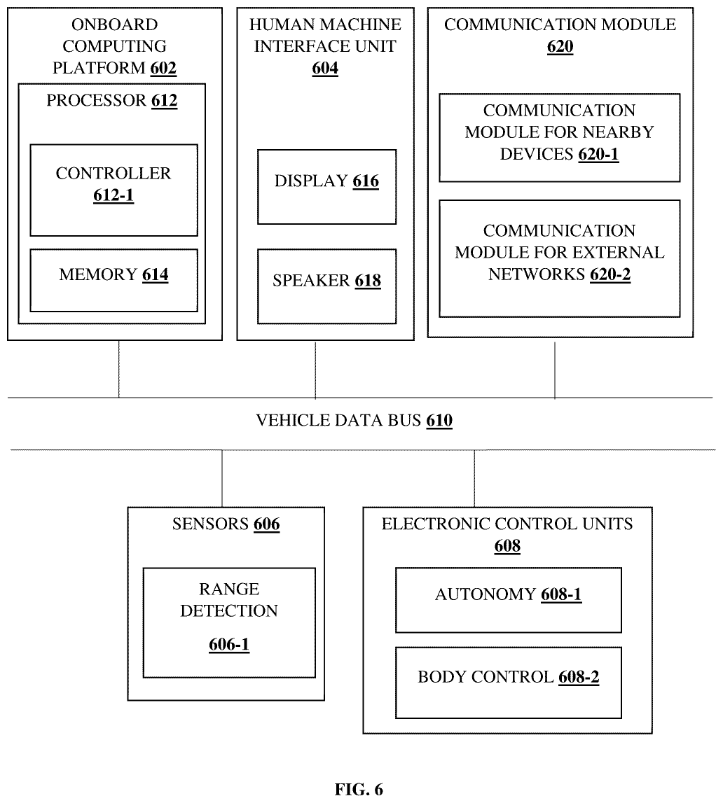

FIG. 6 shows a block diagram of electronic components of a vehicle according to an embodiment. In the illustrated example, the electronic components include an onboard computing platform 602, a human-machine interface (HMI) unit 604, the communication module 620, sensors 606, electronic control units (ECUs) 608, and a vehicle data bus 610.

The onboard computing platform 602 includes a processor 612 (also referred to as a microcontroller unit or a controller) and memory 614. In the illustrated example, processor 612 of the onboard computing platform 602 is structured to include the controller 612-1. In other examples, the controller 612-1 is incorporated into another ECU with its own processor and memory. The processor 612 may be any suitable processing device or set of processing devices such as, but not limited to, a microprocessor, a microcontroller-based platform, an integrated circuit, one or more field programmable gate arrays (FPGAs), and/or one or more application-specific integrated circuits (ASICs). The memory 614 may be volatile memory (e.g., RAM including non-volatile RAM, magnetic RAM, ferroelectric RAM, etc.), non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc.). In some examples, memory 614 includes multiple kinds of memory, particularly volatile memory, and non-volatile memory. Memory 614 is computer readable media on which one or more sets of instructions, such as the software for operating the methods of the present disclosure, can be embedded. The instructions may embody one or more of the methods or logic as described herein. For example, the instructions reside completely, or at least partially, within any one or more of memory 614, the computer readable medium, and/or within the processor 612 during execution of the instructions.

The HMI unit 604 provides an interface between the vehicle and a user. The HMI unit 604 includes digital and/or analog interfaces (e.g., input devices and output devices) to receive input from, and display information for, the user(s). The input devices include, for example, a control knob, an instrument panel, a digital camera for image capture and/or visual command recognition, a touch screen, an audio input device (e.g., cabin microphone), buttons, or a touchpad. The output devices may include instrument cluster outputs (e.g., dials, lighting devices), haptic devices, actuators, a display 616 (e.g., a heads-up display, a center console display such as a liquid crystal display (LCD), an organic light emitting diode (OLED) display, a flat panel display, a solid state display, etc.), and/or a speaker 618. For example, the display 616, the speaker 618, and/or other output device(s) of the HMI unit 604 are configured to emit an alert, such as an alert to request manual takeover, to an operator (e.g., a driver) of the vehicle. Further, the HMI unit 604 of the illustrated example includes hardware (e.g., a processor or controller, memory, storage, etc.) and software (e.g., an operating system, etc.) for an infotainment system that is presented via display 616.

Sensors 606 are arranged in and/or around the vehicle to monitor properties of the vehicle and/or an environment in which the vehicle is located. One or more of the sensors 606 may be mounted to measure properties around an exterior of the vehicle. Additionally, or alternatively, one or more of sensors 606 may be mounted inside a cabin of the vehicle or in a body of the vehicle (e.g., an engine compartment, wheel wells, etc.) to measure properties of the vehicle and/or interior of the vehicle. For example, the sensors 606 include accelerometers, odometers, tachometers, pitch and yaw sensors, wheel speed sensors, microphones, tire pressure sensors, biometric sensors, ultrasonic sensors, infrared sensors, Light Detection and Ranging (lidar), Radio Detection and Ranging System (radar), Global Positioning System (GPS), cameras and/or sensors of any suitable type. In the illustrated example, sensors 606 include the range-detection sensors that are configured to monitor object(s) located within a surrounding area of the vehicle. In the illustrated example, sensors 606, provide interior sensing of the vehicle.

The ECUs 608 monitor and control the subsystems of the vehicle. For example, the ECUs 608 are discrete sets of electronics that include their own circuit(s) (e.g., integrated circuits, microprocessors, memory, storage, etc.) and firmware, sensors, actuators, and/or mounting hardware. The ECUs 608 communicate and exchange information via a vehicle data bus (e.g., the vehicle data bus 610). Additionally, the ECUs 608 may communicate properties (e.g., status of the ECUs, sensor readings, control state, errors, and diagnostic codes, etc.) and/or receive requests from each other. For example, the vehicle may have dozens of the ECUs that are positioned in various locations around the vehicle and are communicatively coupled by the vehicle data bus 610.

In the illustrated example, the ECUs 608 include the autonomy unit 608-1 and a body control module 608-2. For example, the autonomy unit 608-1 is configured to perform autonomous and/or semi-autonomous driving maneuvers (e.g., defensive driving maneuvers) of the vehicle based upon, at least in part, instructions received from the controller 612-1 and/or data collected by the sensors 606 (e.g., range-detection sensors 606-1). Further, the body control module 608-2 controls one or more subsystems throughout the vehicle, such as power windows, power locks, an immobilizer system, power mirrors, child safety seat, vehicle seat belt, air bags, etc. For example, the body control module 608-2 includes circuits that drive one or more relays (e.g., to control wiper fluid, etc.), brushed direct current (DC) motors (e.g., to control power seats, power locks, power windows, wipers, etc.), stepper motors, LEDs, safety systems (e.g., seat belt pretensioner, airbags, etc.), etc.

The vehicle data bus 610 communicatively couples the communication module 620, the onboard computing platform 602, the HMI unit 604, the sensors 606, and the ECUs 608. In some examples, the vehicle data bus 610 includes one or more data buses. The vehicle data bus 610 may be implemented in accordance with a controller area network (CAN) bus protocol as defined by International Standards Organization (ISO) 11898-1, a Media Oriented Systems Transport (MOST) bus protocol, a CAN flexible data (CAN-FD) bus protocol (ISO 11898-7) and/a K-line bus protocol (ISO 9141 and ISO 14230-1), and/or an Ethernet™ bus protocol IEEE 802.3 (2002 onwards), etc.

The communication module 620-1 is configured to communicate with other nearby communication devices. In the illustrated example, communication module 620 includes a dedicated short-range communication (DSRC) module.

V2V communication is typically based on wireless communication protocols such as Dedicated Short-Range Communications (DSRC) or Cellular Vehicle-to-Everything (C-V2X) technology. With V2V communication, vehicles can receive information about potential hazards, such as accidents or road closures, and adjust their behavior accordingly. V2V communication can also be used to support advanced driver assistance systems (ADAS) and automated driving technologies, such as platooning, where a group of vehicles travel closely together using V2V communication to coordinate their movements.

Additionally, or alternatively, the communication module 620-2 includes a cellular vehicle-to-everything (C-V2X) module. A C-V2X module includes hardware and software to communicate with other vehicle(s) via V2V communication, infrastructure-based module(s) via V2I communication and/or more generally nearby communication devices (e.g., mobile device-based modules) via Vehicle-to-Everything (V2X) communication. For example, a C-V2X module is configured to communicate with nearby devices (e.g., vehicles, roadside units, mobile devices, etc.) directly and/or via cellular networks. Currently, standards related to C-V2X communication are being developed by the 3rd Generation Partnership Project.

Further, the communication module 620-2 is configured to communicate with external networks. For example, the communication module 620-2 includes hardware (e.g., processors, memory, storage, antenna, etc.) and software to control wired or wireless network interfaces. In the illustrated example, the communication module 620-2 includes one or more communication controllers for cellular networks (e.g., Global System for Mobile Communications (GSM), Universal Mobile Telecommunications System (UMTS), Long Term Evolution (LTE), Code Division Multiple Access (CDMA)), Near Field Communication (NFC) and/or other standards-based networks (e.g., WiMAX (IEEE 802.16m), local area wireless network (including IEEE 802.11 a/b/g/n/ac or others), Wireless Gigabit (IEEE 802.11ad), etc.). In some examples, the communication module 620-2 includes a wired or wireless interface (e.g., an auxiliary port, a Universal Serial Bus (USB) port, a Bluetooth® wireless node, etc.) to communicatively couple with a mobile device (e.g., a smart phone, a wearable, a smart watch, a tablet, etc.). In such examples, the vehicle may communicate with the external network via the coupled mobile device. The external network(s) may be a public network such as the Internet, a private network such as an intranet, or combinations thereof, and may utilize a variety of networking protocols now available or later developed including, but not limited to, TCP/IP-based networking protocols.

In an embodiment, the communication module is enabled for an autonomous communication, wherein the autonomous communication comprises communication over a period with minimal supervision under different scenarios. The communication module comprises a hardware component comprising, a vehicle gateway system comprising a microcontroller, a transceiver, a power management integrated circuit, an Internet of Things device capable of transmitting one of an analog and a digital signal either wired or wirelessly.

The autonomy unit 608-1 of the illustrated example is configured to perform autonomous and/or semi-autonomous driving maneuvers, such as defensive driving maneuvers, for the vehicle. For example, the autonomy unit 608-1 performs the autonomous and/or semi-autonomous driving maneuvers based on data collected by the sensors 606. In some examples, the autonomy unit 608-1 is configured to operate a fully autonomous system, a park-assist system, an advanced driver-assistance system (ADAS), and/or other autonomous system(s) such as automatic adjustments of the tautness of a vehicle seat belt and/or a child safety seat in the vehicle.

An ADAS is configured to assist a driver in safely operating the vehicle. For example, the ADAS is configured to perform adaptive cruise control, collision avoidance, lane-assist (e.g., lane centering), blind-spot detection, rear-collision warning(s), lane departure warnings and/or any other function(s) that assist the driver in operating the vehicle. To perform the driver-assistance features, the ADAS monitors objects (e.g., vehicles, pedestrians, traffic signals, etc.) and develops situational awareness around the vehicle.

Further, in the illustrated example, controller 612-1 is configured to monitor an ambient environment of the vehicle. For example, to enable the autonomy unit 608-1 to perform autonomous and/or semi-autonomous driving maneuvers, the controller 612-1 collects data that is collected by sensors 606 of the vehicle. In some examples, the controller 612-1 collects location-based data via the communication module 620-1 and/or another module (e.g., a GPS receiver) to facilitate the autonomy unit 608-1 in performing autonomous and/or semi-autonomous driving maneuvers. In operation, according to an embodiment, the communication module 620-1 performs V2V communication with an adjacent vehicle.

In an embodiment, a connection is established between a vehicle and a device. The device is detected by the vehicle control system. The device is detected by exchanging handshaking signals. Handshaking is the automated process for negotiation of setting up a communication channel between entities. The processor sends a start signal through the communication channel in order to detect a device. If there is a device, the processor may receive an acknowledgement signal from the device. Upon receiving the acknowledgement signal, the processor establishes a secured connection with the device. The processor may receive a signal at the communication module from the device. The processor may further automatically determine the origin of the signal. The processor communicatively connects the communication module to the device. Then the processor is configured to send and/or receive a message to and/or from the device. The signals received by the communication module may be analyzed to identify the origin of the signal to determine the location of the device.

In an embodiment, the system is enabled for bidirectional communication. The system sends a signal and then receives a signal/communication. As a step of a method according to the disclosure, a data link between the vehicle and nearby vehicle or any other external device is set up in order to permit data to be exchanged between the vehicle and the nearby vehicle or any other external device in the form of a bidirectional communication. This can take place, for example, via a radio link or a wireless protocol. It is therefore possible for the nearby vehicle or any other external device to receive data from the vehicle or for the vehicle to request data from the nearby vehicle or any other external device.

In an embodiment, bidirectional communication comprises the means for data acquisition and is designed to exchange data bidirectionally with one another. In addition, at least the vehicle comprises the logical means for gathering the data and arranging it to a certain protocol based on the receiving entity's protocol. Initially, a data link for bidirectional communication is set up. The vehicle and the nearby vehicle or any other external device can communicate with one another via this data link and therefore request or exchange data, wherein the data link can be implemented, for example, as a cable link or radio link. Bidirectional communication has various advantages as described herein. In various embodiments, data is communicated and transferred at a suitable interval, including, for example, 200 millisecond (ms) intervals, 100 ms intervals, 50 ms intervals, 20 ms intervals, 10 ms intervals, or even more frequent and/or in real-time or near real-time, in order to allow a vehicle to respond to, or otherwise react to, data. Bidirectional Infrared (IR) communication may be used to facilitate data exchange. The apparatus for the vehicle according to the embodiment that performs bidirectional communication may be by means of a personal area network (PAN) modem. Therefore, a user can have access to an external device using the vehicle information terminal, and can then store, move, and delete the user's desired data.

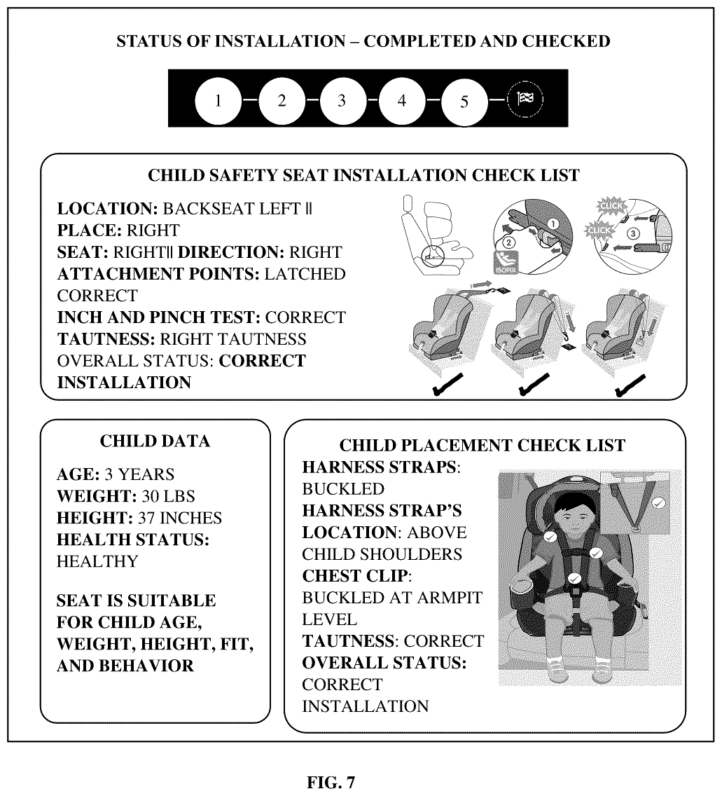

FIG. 7 shows a sample representation of the status of installation on the infotainment system according to an embodiment. A step-by-step instruction may be displayed for each step of the child safety seat installation, child placement and harnessing, and then finally adjusting the child safety seat adjustable parts according to the height and weight of the child. After complete installation and securing the child in the child safety seat a summary status may be displayed on the seat. It may comprise child safety seat installation checklist, child data, child placement checklist which includes child placement, harnessing, and adjusting the adjustable parts according to the child's height and weight. The summary status may also indicate if the child seat is suitable or needs a change based on age, weight, height, fit of the child in a child safety seat, and behavior of the child. In an embodiment, health related status may also be displayed on the infotainment system. The display of the checklist may comprise a combination of audio, video, and an animation or a combination thereof.

According to an embodiment of the system, the processor generates a summary report of the installation, wherein the summary report of the installation comprises a checklist of the installation and corresponding values for the checklist prepared from a real-time data of the installation sensed via a first plurality of sensors of the child safety seat and a second plurality of sensors of the vehicle.

According to an embodiment of the system, the processor generates a summary report of the placing and harnessing of the child in the child safety seat, wherein the summary comprises a checklist of the placing and harnessing of the child in the child safety seat and corresponding values for the checklist prepared from real-time data of the placing and harnessing of the child in the child safety seat sensed via a first plurality of sensors of the child safety seat and a second plurality of sensors of the vehicle.

According to an embodiment of the system, the summary report further comprises results from checking of the child safety seat for a movement of the child safety seat by applying forces at various points for an accuracy of the installation of the child safety seat. According to an embodiment of the system, the processor sends the summary report of the installation via a communication link to a third party for verification and confirmation. According to an embodiment of the system, the third party comprises one or more of a fire station and a police department. According to an embodiment of the system, the system further comprises a data communication module operatively connected to the processor, wherein the data communication module is operable to transmit the summary report collected during the installation of the child safety seat to a remote server for further analysis. According to an embodiment of the system, the system further comprises a data communication module operatively connected to the processor, wherein the data communication module is operable to transmit the real-time data collected during the placing and harnessing of the child in the child safety seat to a remote server for further analysis. According to an embodiment of the system, the information system is further operable to present warnings and alerts to the user based on real-time data collected by the child safety seat. According to an embodiment of the system, the processor is operable to provide decision support to the user by analyzing real-time data of the child and recommending an appropriate child safety seat according to the weight, behavior of the child, and the height of the child.

According to an embodiment of the system, the summary report further comprises results from checking the child safety seat for movement of the child safety seat by applying forces at various points and generates a report on accuracy of the placing and harnessing of the child in the child safety seat. According to an embodiment of the system, the processor is operable to provide the summary report of the placing and harnessing of the child in the child safety seat with a checklist on the information system. According to an embodiment of the system, the system further comprises a data communication module operatively connected to the processor, wherein the data communication module is operable to transmit the summary report collected during the placing and harnessing of the child in the child safety seat to a remote server for further analysis. According to an embodiment of the system, the processor is operable to provide a time-stamped record for each step, which can be accessed and reviewed at a future time by one or more of the user's and a manufacturer of the child safety seat.

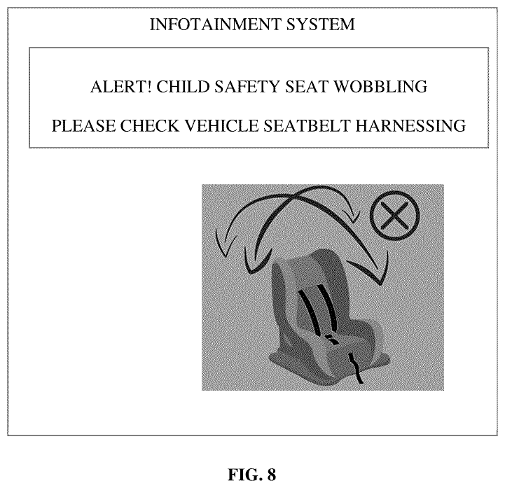

FIG. 8 shows an alert about a child safety seat on a display when the status of the seat installation is changed at any time during a ride in the vehicle. The interior monitoring of the vehicle continues after the installation of the child safety seat. Further the sensors present in the vehicle and the child safety seat may also keep track of the child safety seat and the child's parameters. For example, they may track the movement of the child safety seat when the vehicle is in motion, they may track if the child has opened the harnessing system or come out of the straps, etc. In an embodiment, the monitoring may be via a camera by processing an image or video to analyze the child safety seat and its movements or changes. If the child playing with the seat belt has opened the seat belt or if the child may be feeling very tight or uncomfortable to move due to excessive tautness of the belt, etc., the system monitors and provides an alert. FIG. 8 shows a display of a warning showing that the child safety seat is wobbling. The system may have tracked the movement of the child safety seat and found that the child safety seat may be moving excessively when the vehicle is braking or when the vehicle is in motion. Thus, the system generated a warning on a display of an infotainment system of the vehicle for the user that the child seat needs to be taken care of. It also provides what the user needs to do, for example, “Please check the vehicle seat belt harnessing”. That way the user may be guided by what needs to be checked and fixed to solve the issue.

By the introduction of more advanced interior sensing capabilities, the vehicle is enabled to identify different features in the vehicle compartment such as visual appearance, shape, weight, movement, belt pay out, and other parameters. By use of one or more of these monitored parameters, the vehicle can advise the user on safe configurations of, for example, accessories. It is vital that a child, when traveling in a car, is sitting properly in its seat. Since child safety seats are designed to adapt to different sizes of children, it can be hard to be assured that the child safety seat is adjusted properly to the child currently sitting in the seat.

In an embodiment, the vehicle is provided with sensing capabilities to detect the size of the child, the make and model of the child safety seat, and the child safety seat belt routing when a child is placed in the child safety seat. When all this is detected, the vehicle will be able to determine whether the seat is properly adjusted to the child, or if additional adjustments are required.

There are recommendations for how a child safety seat should properly be adjusted according to child parameters such as height and weight. For instance, it is usually described in a child safety seat manual what is the optimal height of a head rest in relation to the position of the head of the child. Other adjustments based on the child's size may be how much of the child safety seat belt is used (belt payout), if an inlay is used (additional components used in some child safety seats up until a certain age), to secure a proper space for the legs, angle of child safety seat, etc. In an embodiment, interior sensing will be enabled and by combining different methods for sensing, for instance (but not limited to): shape recognizing camera/radar, weight sensors in seat bolsters, force measurement sensors, RFID tags, etc., the vehicle is enabled to detect one or more of a length of the child, a weight of the child, a make and model of the child safety seat, position of adjustable parts in the child safety seat, for example, head rest, belt, leg room, child safety seat angle, etc.

Based on the detected information about the child and the child safety seat, the vehicle will be able to determine the optimal adjustment of the specific child safety seat for the specific child. And through information from adjustable parts of the child safety seat, the system can judge whether the child safety seat is adjusted properly. If not, it can instruct the adult occupants in the vehicle as to how the child safety seat should be adjusted to best fit the child; for instance, raise the headrest by one step, tighten the seat belt, create more leg room, etc. In another embodiment, the adjustments are enabled to be performed automatically by the vehicle control system.

In one aspect, the system guides on the placement of the child safety seat, recognizing the seat, and then to check whether the child safety seat is placed properly. In another aspect, the system suggests adjustments to the child safety seat installation and surrounding areas for legroom, etc., based on a child inside the child seat. Based on the length and the weight of the child, adjustments to the child seat installation are made. The adjustments are based on the way a user drives the vehicle, a place where the driver is planning to drive, a situation under which the driver is driving, a child's behavior (such as very active, not moving much, etc.), age of the child, calmness of the child, clothing of the child. The system determines whether a seat in front of the child safety seat is close and not providing enough legroom for the child in the child safety seat. The system then adjusts the seats in front of the child safety seat automatically or may suggest to the user and guide him on proper adjustment. In an embodiment, the system may suggest a placement of the child safety seat based on other occupants in the vehicle. An Artificial Intelligence (AI) and Machine Learning (ML) models associated with the vehicle can recognize a child that is getting into the child safety seat, learn the parameters and behavior of the child and, based on the parameters and behavior of the child, may determine any adjustments that need to be made to the child safety seat and the installation of the child safety seat.

According to an embodiment of the system, a face detection algorithm is used to detect a child. According to an embodiment of the system, a camera is operable to capture an image and/or video of the child in the child safety seat. According to an embodiment of the system, the image and/or the video is analyzed using a computer vision algorithm comprising pattern recognition techniques to detect a human, facial features of the human for at least one of an eye, a nose, a mouth, and an outline of a face, an ear, a hair.

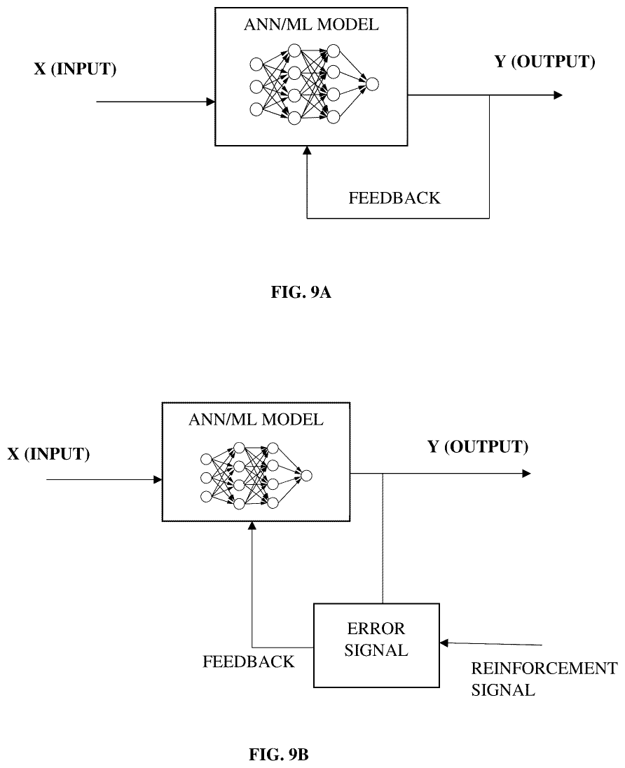

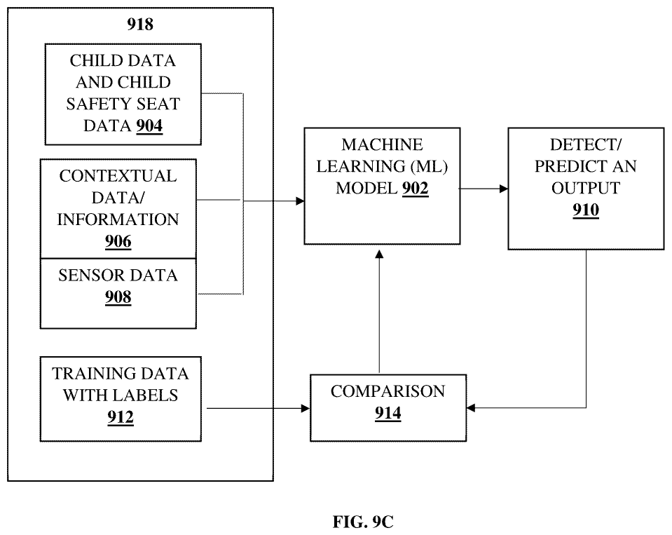

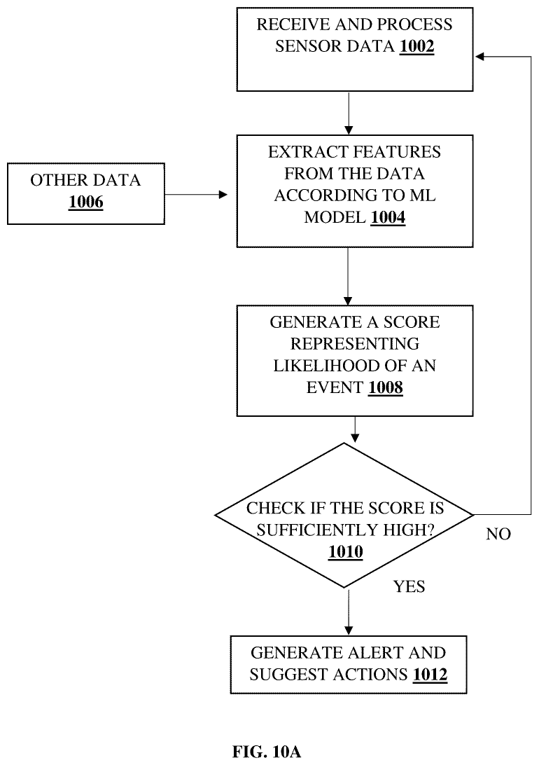

FIG. 9A shows a structure of the neural network/machine learning model with a feedback loop. Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The model may learn from the data. Learning may be supervised learning and/or unsupervised learning and may be based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer may predict or detect an anomaly in the child safety seat installation, child placing and harnessing and adjustment of the child safety seat adjustable parts and provide an alert based on the input data, such as a child image, an image of the child safety seat installed, a driving behavior, a child behavior, a movement of the child safety seat, a path taken, environmental factors (such as snow, rain), a situation under which the driver is driving the vehicle. In an embodiment, the system analyzes child size, weight, age, a fit of the child on the child safety seat, and behavior to output a suitability of the child safety seat for the child.

In an embodiment, ANN's may be a Deep-Neural Network (DNN), which is a multilayer tandem neural network comprising Artificial Neural Networks (ANN), Convolution Neural Networks (CNN) and Recurrent Neural Networks (RNN) that may recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback. The variations of weights in the hidden layer(s) will be adjusted to fit the expected outputs better while training the model. This will allow the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labeled data and concepts, after a while the models' performance may decline while adding new, unlabeled input due to many reasons which include, but not limited to, concept drift, recall precision degradation due to drifting away from true positives, and data drift over time. A feedback loop to the model keeps the AI results accurate and ensures that the model maintains its performance and improvement, even when new unlabeled data is assimilated. A feedback loop refers to the process by which an AI model's predicted output is reused to train new versions of the model.

Initially, when the AI/ML model is trained, a few labeled samples comprising both positive and negative examples of the concepts (e.g., incorrect installations including harness not proper, crooked belt, buckle not properly fixed, chest clip not proper, child legs are not going through the belt system properly, improper child behavior like attempt to unbuckle or attempt to exit the child safety seat, etc.) are used that are meant for the model to learn. Afterward, the model is tested using unlabeled data. By using, for example, deep learning and neural networks, the model may then make predictions on whether the desired concept/s (e.g., crooked belt, loose harnessing, tight harnessing, a height of the child, an adjustable part in the seat, improper child behavior, etc.) are in unlabeled images. Each image is given a probability score where higher scores represent a higher level of confidence in the models' predictions. Where a model gives an image a high probability score, it is auto labeled with the predicted concept. However, in the cases where the model returns a low probability score, this input may be sent to a controller (maybe a human moderator) which verifies and, as necessary, corrects the result. The human moderator may be used only in exceptional cases. The feedback loop feeds labeled data, auto-labeled or controller-verified, back to the model dynamically and is used as training data so that the system may improve its predictions in real-time and dynamically.

FIG. 9B shows a structure of the neural network/machine learning model with reinforcement learning. The network receives feedback from authorized networked environments. Though the system is similar to supervised learning, the feedback obtained in this case is evaluative, not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. The model may be fed with new data for testing, hence by feeding the model with data it has already predicted over, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time.

FIG. 9C shows an example block diagram for child size assessment in a child safety seat using a machine learning model. The machine learning model 902 may take as input any data associated with the child safety seat and a child and learn to identify features within the data that are predictive for installation instructions such as incorrect installations, belt tautness, chest clip position, child's height based on sitting posture, identify the child, child posture, child gesture, child stature, improper child behavior, a fit of the child in child safety seat, historical data of the child in the child seat during various rides, etc. The training data sample may include, for example, the child data and child safety seat data 904, such as the child safety seat size, child safety seat dimensions, child safety seat type, child safety seat model, child safety seat weight, child safety seat length, etc., similar child size, child height, child weight, child behavior, etc. In an embodiment, it relates to systems and methods that identify a type of child safety seat and its features in real time using an on-board camera and/or other sensors. This child safety seat identification and feature information are transmitted to the cloud, where the vehicle identification is coupled with available child safety seat models in the case of a previously identified child safety seat of a similar class and build having similar characteristics. Subsequently, the information is used to compute/estimate/access a child safety seat model, features, and installation instructions and usage limits. The systems and methods of the present disclosure may also provide data analytics information that may be used later to improve child safety seat installation accuracy; child placement and harnessing accuracy; child safety seat suitability; and adjusting the adjustable parts of the child safety seat according to the one or more of child height, weight, and behavior. When a child is in the child safety seat, sensors and cameras of the vehicle and a facial recognition algorithm, or the like, may be used to detect the child's characteristics and behavior. Child behavior may include and may be measured via excessive movements, no movements, a force or pressure exerted on the seat belt etc.

In an embodiment, the training data sample may also include current contextual information 906 relating to the surrounding environment. This may include, for example, location of the vehicle, current weather conditions, temperature, time of day, traffic conditions in the region, driver behavior, child behavior, number of children in the vehicle, etc. The system may also garner contextual information 906 from a device associated with the vehicle. For example, through an application installed on the device, such as Google® maps and location services, the system may know the vehicle details.

Real-time sensor data 908 may include, for example, video, image, audio, infrared, temperature, 3D modeling, and any other suitable types of data that capture the current state in and around the vehicle. In an embodiment, the real-time sensor data may be processed using one or more machine learning models 902 trained and based on similar types of data to predict real-time features of the installation requirements and anomalies/mistakes/improper installations of the child safety seat for a given model and make. The real-time features may include, for example, the child safety seat size, shape, dimensions, etc. Current information about the child safety seat and the child may be used for generating guiding instructions, installation requirements, specific concentration areas based on common installation mistakes, analysis for suitability of the child safety seat, etc. For example, currently detected sensor data and/or previously known information about the child safety seat class and/or installation requirements may be used to detect an improper installation and an unsuitability or a suitability of the child safety seat.

Any of the aforementioned types of data (e.g., child data and child safety seat data 904, contextual information 906, sensor data 908, or any other data) may correlate with the requirements for child safety seat installation and usage limits, and such correlations may be automatically learned by the machine learning model 902. In an embodiment, during training, the machine learning model 902 may process the training data sample (e.g., child data and child safety seat data 904, contextual information 906) and, based on the current parameters of the machine learning model 902, detect or predict an output 910 which may be a requirement of specific tautness for the seat belt of the child safety seat, an alert for improper installation of seat belt, an undesirable behavior of the child in the child safety seat that jeopardizes safety or an unsuitability of the child safety seat for the child. The detection or prediction of installation requirements, usage limits, and suitability of the child safety seat may depend on the training data with labels 912 associated with the training data sample 918. Predicting an improper seat belt tautness refers to predicting a future event based on past and present data and most commonly by analysis, estimation of trends or data patterns. Prediction or predictive analysis employs probability based on the data analyses and processing. Detection of seat belt tautness refers to an onset of the event and the system detecting the same. Predicted events may or may not turn into a reality based on how the turn of events occur. In an embodiment, during training, the detected event at 910 and the training data with labels 912 may be compared at 914. For example, comparison 914 may be based on a loss function that measures a difference between the detected event 910 and the training data with labels 912. Based on the comparison at 914 or the corresponding output of the loss function, a training algorithm may update the parameters of the machine learning model 902, with the objective of minimizing the differences or loss between subsequent predictions or detections of the event 910 and the corresponding labels 912. By iteratively training in this manner, the machine learning model 902 may “learn” from the different training data samples and become better at detecting various collision events at 910 that are similar to the ones represented by the training labels at 912. In an embodiment, the machine learning model 902 is trained using data which is specific to a type of target for which the model is used for detecting various improper installations, improper placement of child and harnessing of the child, improper child seat adjustments. In an embodiment, the machine learning model 902 is trained using data which is general to the child safety seat types and is used for detecting various improper installations, improper placement of child and harnessing of the child, improper child seat adjustments, undesirable behavior of the child, and unsuitability of a child safety seat.

Using the training data, a machine learning model 902 may be trained so that it recognizes features of input data that signify or correlate to certain event types. For example, a trained machine learning model 902 may recognize data features that signify the likelihood of an improper child safety seat installation, an unsuitability of the child safety seat for the child, undesirable behavior of the child, etc. Through training, the machine learning model 902 may learn to identify predictive and non-predictive features and apply the appropriate weights to the features to optimize the machine learning model's 902 predictive accuracy. In embodiments where supervised learning is used and each training data sample 918 has a label 912, the training algorithm may iteratively process each training data sample 918 (including child data and child safety seat data 904, contextual information 906, sensor data 908, or any other data), and generate a prediction of installation requirements 910 based on the model's 902 current parameters. Based on the comparison at 914 results, the training algorithm may adjust the model's 902 parameters/configurations (e.g., weights) accordingly to minimize the differences between the generated prediction of installation requirements 910 and the corresponding labels 912. Any suitable machine learning model and training algorithm may be used, including, e.g., neural networks, decision trees, clustering algorithms, and any other suitable machine learning techniques. Once trained, the machine learning model 902 may take input data associated with a child safety seat and output one or more predictions that indicate installation requirements and alerts for various improper installations.

FIG. 10A shows an example flow chart for detecting an improper installation of a child safety seat and/or placing and harnessing the child in the child safety seat using a machine learning model. The system may receive data associated with sensor output(s) from one or more sensors in the vehicle as shown at 1002. Any type of sensor may be used to gather data pertaining to the vehicle. A sensor output may be, for example, images, videos, audios, LiDAR measures, infrared measures, temperature measures, GPS data, or any other information measured or detected by sensors. In an embodiment, a sensor output may be the result of one or more sensors capturing environmental information associated with the surroundings of the vehicle, which may include traffic at the location, path details, child behavior, child state, driver behavior, etc. For example, sensor output may be associated with a passenger in the vehicle including the driver. In an embodiment, data associated with sensor output may include the raw sensor output (e.g., the images, videos, audios, etc.) and/or data derived from processing the sensor output. As an example, derivative data from image, video or LiDAR data may include data representing identified objects, motion, gestures, facial expressions, posture, eye position, etc. The system may receive any data associated with the sensor output from sensors, including raw sensory output and/or any derivative data. In an embodiment, the system may process the received data and identify any actionable event of interest using a machine learning model, trained using a set of training data. The system may also receive any other data as shown at 1006, wherein other data could be weather data, manual input, a manufacturers' website data relating to child safety seats, other safety instructions by various organizations, etc.

As shown at 1004, the system may extract features from the received data according to a machine learning model. The machine learning model is able to automatically do so based on what it learned during the training process. In an embodiment, appropriate weights that were learned during the training process may be applied to the features. Extracting features include, but are not limited to, installation checks at various areas of interest on the child safety seat, for example, a belt crookedness or straightness, proper buckling, etc.

At step 1008, the machine learning model, based on the features of the received data, may generate a score representing a likelihood or confidence that the received data is associated with a particular event type, e.g., an improper installation where the belt tautness is not correct, where the child seat may be moving after installation, the belt is not routed properly, etc.

As shown at 1010, the system may determine whether the score is sufficiently high relative to a threshold or criteria to warrant certain action. If the score is not sufficiently high, thus indicating a false-positive, the system may return to step 1002 and continue to monitor subsequent incoming data. On the other hand, if the score is sufficiently high, then at step 1012 the system may generate an appropriate alert and/or determine an appropriate action/response. In an embodiment, the system may send alerts to appropriate recipients based on the detected event types. For instance, an alert is generated in the vehicle on the infotainment system saying that the belt of the child safety seat is not properly buckled, or the harness is too loose, and the child may come out of the belt, etc.

In an embodiment, the system may repeat one or more steps of the method of FIG. 10A, where appropriate. In an embodiment, the steps 1002 to 1012 may be performed by the system, any combination of those steps may be performed by any other computing system, for example, a remote network or a cloud network. In an embodiment, where machine learning models are used for making such determination, the system may transmit a trained machine learning model to the computing system in the vehicle to allow event-detection to occur locally. This may be desirable since sensor data may be overly large to transmit to the remote system in a timely fashion.

FIG. 10B shows example steps for determining suitability of a child safety seat based on child's size, weight, age, fit, and behavior, according to an embodiment. The steps for determining suitability of a child safety seat comprise: collect data 1022, preprocess data 1024, extract features and train a ML model 1026, analyze behavior 1028, analyze size and weight 1030, analyze seat fit 1032, consider age 1034, analyze using decision logic 1036, generate alerts and notifications 1038, and log data and provide feedback 1040.

Data Collection unit is operable to collect data 1022 on child's age, height, weight and further collect seat-related data (for e.g., seat size, harness settings). The unit further monitors child behavior during vehicle rides. The data collection unit comprises a sensor module. According to an embodiment of the system, the sensor module further comprises one or more of a seat belt sensor, a position sensor, a pressure sensor, a weight sensor, a motion sensor, an infrared sensor, a lidar sensor, an optical sensor, a moisture sensor, a temperature sensor, a laser sensor, an ultrasonic sensor, a level sensor, and a time-of-flight sensor. According to an embodiment of the system, the radar sensor is a millimeter-wave (mmWave) radar sensor.

According to an embodiment of the system, the image sensor is a camera comprising a computer vision module. According to an embodiment of the system, the sensor module is operable to collect a first real-time data about the child safety seat, wherein the first real-time data comprises one or more of a make, a model, a shape, and a size of the child safety seat. According to an embodiment of the method, the sensor module is operable to collect second real-time data about the child, wherein the second real-time data comprises one or more of a height of the child, a weight of the child, a movement of the child, a posture of the child, a gesture of the child, a health parameter of the child, and a face detection of the child.

According to an embodiment of the method, the image sensor is a camera comprising a computer vision module. According to an embodiment of the method, the camera is operable for capturing a video in real-time and enabling continuous monitoring. According to an embodiment of the method, the video is analyzed for detecting a behavior of the child, wherein the behavior comprises one or more of an attempt to unbuckle a harness of the child safety seat, a restlessness of the child seated in the child safety seat, an attempt to exit the child safety seat, and a legroom available for the child in the child safety seat.

According to an embodiment of the method, the sensor module is operable to collect first real-time data about the child safety seat, wherein the first real-time data comprises one or more of a make, a model, a shape, and a size of the child safety seat. According to an embodiment of the system, the sensor module is operable to collect second real-time data about the child, wherein the second real-time data comprises one or more of a height of the child, a weight of the child, a movement of the child, a posture of the child, a gesture of the child, a health parameter of the child, and a face detection of the child.

The Data Collection unit collects data from various sensors and cameras placed within the vehicle. This data may include video footage, accelerometer data, pressure sensor readings, or any other relevant information. Interior sensing in vehicles relies on various sensors to monitor and detect different aspects of the vehicle's interior and its occupants. Ultrasonic sensors emit high-frequency sound waves and measure their reflection to detect the presence and location of occupants in the vehicle. They can be used to trigger seat belt warnings. Similarly, Infrared (IR) sensors may also be used to detect the presence of occupants by measuring changes in infrared radiation within the vehicle cabin. Seat belt buckle sensors that are integrated into the seatbelt buckle are used to detect whether a seatbelt is fastened or unfastened. Seat occupancy pressure sensors are placed within the vehicle seats and detect changes in pressure to determine if a seat is occupied. Weight sensors integrated into the vehicle's seats can detect the presence and weight of occupants in specific seating positions. Capacitive sensors are used in touch-sensitive buttons, screens, and surfaces within the vehicle's interior. They detect the touch or proximity of a user's hand or finger to detect and/or control various functions. Cameras equipped with 3D depth-sensing technology can detect hand gestures and movements of occupants. These sensors may be used for gesture-based control or may be used for analysis based on gestures. Interior cameras integrated into the rearview mirror, dashboard, in front of seats capturing an occupant, may be used to detect an occupant and monitor them for various purposes including their behavior. Microphones in the vehicle's interior capture voice from occupants, enabling voice-activated control or voice related monitoring activities. Temperature and Humidity Sensors monitor temperature and humidity levels within the cabin to ensure passenger comfort. Cabin air quality sensors such as gas and odor sensors detect the presence of harmful gases or odors within the vehicle, such as carbon monoxide or volatile organic compounds.

A Preprocessing unit is operable to preprocess data 1024 where the unit cleans and normalizes the data and annotates the data with labels, e.g., “within limits” or “behavior concerns”. It processes and cleans the data to remove noise and irrelevant information. It further annotates the data with labels indicating the state of the child (e.g., correctly secured, harness unbuckled, fidgeting, etc.).

A Model Training unit is operable to extract the features from the data and train a machine learning model 1026 by using annotated data. It includes extracting relevant features from the data that could indicate child behavior or safety seat-related issues. Features could include motion patterns, gestures, posture, movements, pressure changes, and/or facial expressions. Annotated data is used to train a machine learning model (e.g., a deep neural network, a decision tree, a random forest, or a support vector machine). The model learns to recognize patterns associated with problematic behavior. According to an embodiment of the method, the machine learning module comprises one or more of an object detection algorithm, a facial recognition algorithm, a pose estimation algorithm, a gesture recognition algorithm, a voice recognition algorithm, a gait recognition algorithm, and an activity recognition algorithm. According to an embodiment of the method, the machine learning module comprises one or more of a You Only Look Once (YOLO) algorithm, a Haar cascade classifier, an OpenPose algorithm, a Convolution Neural Network, a Recurrent Neural Network, Support vector machines.

According to an embodiment of the method, the machine learning module is operable for detecting and categorizing a child behavior from the second real-time data. According to an embodiment of the method, the second characteristic of the child comprises the height of the child, the weight of the child, the age of the child, the behavior of the child, and the fit of the child in the child safety seat. According to an embodiment of the system, the machine learning module is operable for detecting and categorizing the child behavior from the second real-time data.

A Behavior Analysis unit is operable to analyze behavior 1028 of the child during car rides by considering factors such as restlessness, discomfort, and attempts to unbuckle. Such sensor data and/or video footage, in real-time, are input to the trained AI/ML model. The AI/ML model will output predictions regarding the child's behavior and the safety seat's condition. AI/ML model predicts and identifies any concerning behavior or issues related to the child's interaction with the child safety seat. Examples of concerning behavior could include continuous attempts to unbuckle the harness, extended periods of restlessness, or attempts to exit the safety seat while the vehicle is in motion. It may include posture analysis to identify issues with leg and arm positions and whether enough leg room is available for the child to move around with limbs comfortable even while harnessed. According to an embodiment of the system, the machine learning module comprises one or more of an object detection algorithm, a facial recognition algorithm, a pose estimation algorithm, a gesture recognition algorithm, a voice recognition algorithm, a gait recognition algorithm, and an activity recognition algorithm. According to an embodiment of the system, the machine learning module comprises one or more of a You Only Look Once (YOLO) algorithm, a Haar cascade classifier, an OpenPose algorithm, a Convolution Neural Network, a Recurrent Neural Network, and Support Vector Machines.

Size and Weight Analysis unit is operable to analyze size and weight 1030 of the child and evaluate child's height and weight in relation to seat specifications and compare measurements with seat manufacturer's guidelines. According to an embodiment of the method, the specification information of the child safety seat comprises one or more of type of the child safety seat, a positioning of the child safety seat, an orientation of the child safety seat, a feature of the child safety seat, a height limit, a weight limit, an age limit; and wherein the orientation of the child safety seat comprises one of a rear facing child safety seat, a front facing child safety seat, a convertible child safety seat, and an all-in-one child safety seat.

Ultrasonic sensors emit high-frequency sound waves and calculate the distance to an object by measuring the time it takes for the sound waves to bounce back. Placing ultrasonic sensors within the car seat allows for the measurement of the child's height in real-time. Infrared (IR) sensors are another option, as they detect the child's presence and position by analyzing how IR beams are interrupted. Often, a combination of sensors, such as ultrasonic and pressure sensors, are used to improve accuracy.

Load cells or pressure sensors integrated into the car seat can be used in estimating the child's height. These sensors measure weight distribution and pressure exerted by the child, which can then be used to estimate their height and position. Additionally, car seats may be equipped with smart fabrics that contain embedded sensors. These sensors detect pressure and deformation caused by the child's presence, providing valuable data for estimating the child's height.

Cameras placed within the vehicle are used to estimate the child's height. Computer vision algorithms analyze images or video footage of the child in the car seat to determine their posture and size and estimate their height. Multi-sensor fusion is a common approach, where data from multiple sensors, such as ultrasonic sensors, pressure sensors, and cameras, is combined to improve measurement accuracy.

Machine learning algorithms are also increasingly utilized to process data from various sensors and cameras. These algorithms adapt and learn from different seating scenarios and positions, enhancing measurement accuracy over time. These algorithms are operable for ensuring that the child is correctly secured in a car seat that is suitable for their specific characteristics, including height, weight, and age.

The Seat Fit Analysis unit is operable to analyze seat fit 1032 to determine how well the child fits in the seat by using harness positions, snugness, limb positions, etc., to assess the adequacy of harness adjustments.

The Seat fit analysis unit evaluates how well a child fits in a car seat, including assessing harness positions, snugness, and limb positions, by integration of sensors and control systems. Harness position sensors are strategically placed within the child safety seat and are designed to detect the height and placement of the harness straps on the child's shoulders and torso. By measuring the distance between the child's shoulders and the harness straps, these sensors help ensure that the straps are correctly positioned for optimal safety. Additionally, they can assess the tightness of the harness to prevent it from being too loose or too tight. Snugness and tightness sensors, including load cells or pressure sensors, are used in measuring the force exerted by the harness straps on the child's body. These sensors ensure that the harness is snug and secure, providing an appropriate level of restraint without discomfort. Tension sensors can also gauge the tension in the harness straps and offer real-time feedback to the control system regarding the straps' tightness. Limb position sensors may employ infrared sensors and/or cameras, monitor the child's limb positions within the child safety seat. They help identify if the child's limbs are correctly positioned, have enough room for movement, and are not interfered with by the harness or other safety components. All sensor data is collected and integrated into a central control system of the vehicle. This control system analyzes the data to assess whether the child is adequately secured in the seat. It considers factors such as harness height, snugness, and limb positions. In some embodiments, the control system can even make real-time adjustments, such as tightening the harness straps if they are deemed too loose. For continuous monitoring and improvement, the data regarding harness adjustments and child positioning is logged into the system memory.

The Age Consideration unit is operable to consider age 1034 of the child in relation to child safety seat recommendations. Some child safety seats provide age-based guidelines in addition to height and weight.

One of the ways to gather information about the child's age is through user input. Drivers or caregivers can manually input the child's age into the vehicle's system. This input can be used to tailor certain safety features or recommendations based on the child's age, such as adjusting airbag deployment settings or seat belt reminders. Some vehicles are equipped with weight and size sensors integrated into the seats. These sensors can estimate the child's weight and size, and while age isn't directly measured, it may be approximated within a general range. Certain weight and size ranges are often associated with specific age groups, allowing the system to estimate the child's age within a certain range.

Advanced camera systems within the vehicle are also leveraged. While not directly determining age, these systems can capture images or video footage of the child in the car seat. This data can then be used to assess the child's physical development, which might provide an estimate of age within a certain range. Facial detection technology, in particular, can analyze facial features and potentially provide age-related cues. User profiles can further assist in age estimation. In vehicles with personalized user profiles, the child's age can be inferred from the user profile associated with the car seat. Social media data, photographs, birthday information may further be used to estimate the age of the child in the child safety seat.

The Decision Logic unit is operable to analyze using decision logic 1036 to determine if the child has likely outgrown the seat based on size, weight, fit, age, and behavior by considering seat-specific guidelines.

The Decision Logic unit within a child safety seat monitoring system is responsible for making informed determinations about whether a child has likely outgrown their current car seat. It accomplishes this by carefully analyzing several key factors, including size, weight, fit, age, and behavior, all while adhering to seat-specific guidelines provided by the car seat manufacturer.

Size and weight are among the basic considerations for the decision logic. The decision logic unit evaluates the child's physical dimensions and weight in relation to the specifications outlined by the car seat manufacturer. If the child's size or weight exceeds the recommended limits for the specific child safety seat model, the decision logic unit will raise a concern. For instance, if the car seat is designed for children up to 40 pounds, and the child weighs 45 pounds, the decision logic unit will likely flag this as an issue.

Fit and harness analysis is another crucial aspect. The decision logic unit examines how well the child fits within the car seat and the appropriateness of harness adjustments. This assessment includes evaluating the height and placement of the harness straps and the snugness of the harness. If the harness straps are found to be too low, too high, or too loose for the child's size, the decision logic unit may recommend transitioning to a different car seat.

Age is one of the factors in determining seat suitability. Car seats may have age-based guidelines in addition to height and weight limits. The decision logic unit interprets the child's age and compares it to the manufacturer's recommendations. For example, if a particular car seat is intended for children under two years old, and the child is three, the decision logic unit may suggest that it's time to switch to a seat designed for older children.

Behavior analysis is an additional layer of assessment. The decision logic unit considers the child's behavior during car rides, as it can provide insights into comfort and safety. Restlessness, discomfort, or attempts to unbuckle the harness may indicate that the child no longer feels secure in the seat. These behavioral cues are used for decision-making, as they reflect the child's well-being and overall experience during car journeys.

The decision logic unit accounts for seat-specific guidelines provided by the car seat manufacturer. Different car seat models may come with distinct recommendations and requirements, such as extended rear-facing options or differing height and weight limits. The decision logic unit ensures that these seat-specific guidelines are accurately interpreted and applied to the assessment process.

Decision logic may further consider recommending a new child safety seat even when the child is within the height, weight, and age limits of their current seat. The decision to recommend a new child safety seat may be influenced by various factors and circumstances. Child safety seats typically come with an expiration date, which is often printed on the seat itself. Even if a child is within the height, weight, and age limits, if the seat has reached its expiration date, it's recommended to replace it. This is because the materials in the seat can degrade over time and may not provide the same level of protection. If the current child safety seat has been involved in a significant crash, it may no longer provide the same level of protection, even if it appears undamaged. Many manufacturers recommend replacing the seat after a moderate to severe crash, as the structural integrity of the seat could be compromised. If the child safety seat is subject to a recall or if a safety defect is identified, it is crucial to replace it with a safe and compliant seat. Manufacturers or regulatory agencies may issue recalls if a safety issue is identified which are monitored and notified. The vehicle may connect to the manufacturer's website or any public website where such information is updated and accessed to notify the user. Child safety seat regulations and guidelines may change over time. If new guidelines or regulations are introduced that recommend different types or designs of seats for a child's age or size, it may be advisable to replace the seat to ensure compliance with the latest safety standards.

Sometimes, a child may outgrow their current seat not in terms of height, weight, or age but in terms of comfort and fit. If the child seems uncomfortable, restless, or has difficulty fitting properly in the seat, it may be time to consider a larger or more suitable seat.

Changes in the family's lifestyle or vehicle may also warrant a change in the child safety seat. For example, if a family switches to a smaller vehicle or one with specific seating configurations, they may need a different type of child safety seat to accommodate these changes. If a child safety seat is being reused from one child to another within a family or passed down from another source, it's essential to ensure that it meets current safety standards and hasn't expired.

The decision logic unit plays a pivotal role in ensuring child safety during car rides. It evaluates multiple factors, including size, weight, fit, age, behavior, and manufacturer-specific guidelines, to make informed recommendations about the suitability of the child's current car seat. These recommendations are intended to guide caregivers in providing the best possible protection for their child during travel. Implementing decision logic helps to determine whether a safety seat change is necessary based on the detected behavior. The unit further considers factors such as the severity and frequency of detected issues, the age and size of the child, and whether the issues compromise safety.

According to an embodiment of the method, determining the suitability comprises information that indicates at least one of continuing use of the child safety seat, a replacement for the child safety seat, and an upgradation of the child safety seat.

The Alert and Notification unit is operable to generate alerts and notifications 1038 and trigger alerts when the algorithm determines that the child has likely outgrown the seat or displays concerning behavior. It may further suggest actions to caregivers such as recommending a new seat, and/or addressing behavior issues. If the algorithm determines that a safety seat change is necessary, it can trigger alerts or notifications to the vehicle's occupants or caregivers. The alert might suggest inspecting and adjusting the safety seat, educating the child about safe behavior, or considering a different seat model. According to an embodiment of the method, the method is further operable to provide an alert signal upon determining a necessity for at least one of the replacement of the child safety seat and the upgradation of the child safety seat. According to an embodiment of the method, the alert signal comprises one or more of a text message, a visual cue, a sound alert, a tactile cue, and a vibration.

According to an embodiment of the system, the system further provides an alert signal upon determining a necessity for at least one of the replacement of the child safety seat and the upgradation of the child safety seat. According to an embodiment of the system, the alert signal comprises one or more of a text message, a visual cue, a sound alert, a tactile cue, and a vibration. According to an embodiment of the system, the system further recommends a position for the child safety seat and a direction of installation of the child safety seat. According to an embodiment of the system, the system further comprises a display configured to display a message, wherein the message comprises a position of the child safety seat, a direction of the child safety seat, and the suitability of the child safety seat.

The Data Logging and Feedback unit is operable to log data and provide feedback 1040. Logging of data from various sensors and the analysis along with inferences and conclusions are stored in the system memory. Further the system gathers feedback from caregivers via surveys and questionnaires. Continuous data logging and feedback is considered to improve the algorithm's accuracy and reliability over time.

In an embodiment, the system may comprise a cyber security module. In one aspect, a secure communication management (SCM) computer device for providing secure data connections is provided. The SCM computer device includes a processor in communication with memory. The processor is programmed to receive, from a first device, a first data message. The first data message is in a standardized data format. The processor is also programmed to analyze the first data message for potential cyber security threats. If the determination is that the first data message does not contain a cyber security threat, the processor is further programmed to convert the first data message into a first data format associated with the vehicle environment and transmit the converted first data message into a first data format associated with the vehicle environment and transmit the converted first data message to the communication module using a first communication protocol associated with the negotiated protocol.

According to an embodiment, secure authentication for data transmissions comprises, provisioning a hardware-based security engine (HSE) located in the cyber security module, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the cyber security module with other computing system subjects (e.g., communication module) to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

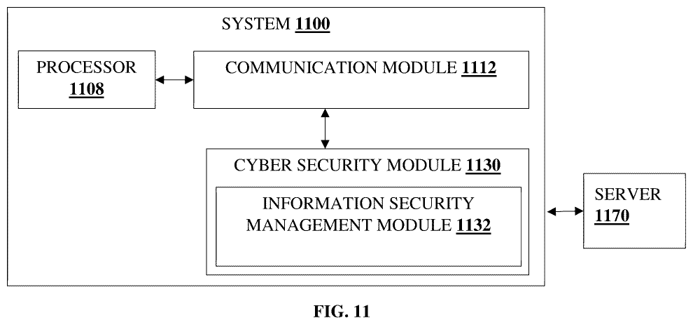

FIG. 11 shows the block diagram of the cyber security module according to an embodiment. In an embodiment, FIG. 11 shows the block diagram of the cyber security module. The communication of data between the system 1100 and the server 1170 through the communication module 1112 is first verified by the information security management module 1132 of the cybersecurity module 1130 before being transmitted from the system to the server or from the server to the system. The information security management module is operable to analyze the data for potential cyber security threats, to encrypt the data when no cyber security threat is detected, and to transmit the data encrypted to the system or the server. System 1100 comprises a processor 1108.

In an embodiment, the cyber security module further comprises an information security management module providing isolation between the system and the server. The information security management module is operable to receive data from the communication module. The information security management module exchanges a security key at the start of the communication between the communication module and the server. The information security management module receives a security key from the server. The information security management module authenticates an identity of the server by verifying the security key. The information security management module analyzes the security key for potential cyber security threats. The information security management module negotiates an encryption key between the communication module and the server. The information security management module receives the encrypted data. The information security management module transmits the encrypted data to the server when no cyber security threat is detected.

In an embodiment, the information security management module is operable to exchange a security key at the start of the communication between the communication module and the server. The information security management module receives a security key from the server. The information security management module authenticates an identity of the server by verifying the security key. The information security management module analyzes the security key for potential cyber security threats. The information security management module negotiates an encryption key between the communication module and the server. The information security management module receives encrypted data. The information security management module decrypts the encrypted data and performs an integrity check of the decrypted data. The information security management module transmits the decrypted data to the communication module when no cyber security threat is detected.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

1-55. (canceled)

56. A system comprising:
a sensor module comprising a radar sensor and an image sensor;
a processor; and
a memory communicably coupled to the processor comprising computer-readable instructions that when executed by the processor cause the processor to:
sense, at least a portion of an interior of a vehicle, via the sensor module;
acquire, information of the interior of the vehicle, via the sensor module;
determine, using the information of the interior of the vehicle via a machine learning module, a first characteristic of a child safety seat positioned in the interior of the vehicle;
retrieve, using the first characteristic of the child safety seat positioned in the interior of the vehicle, a specification information of the child safety seat;
determine, using the information of the interior of the vehicle via the machine learning module, a second characteristic of a child positioned in the child safety seat;
compare, the second characteristic of the child with the specification information of the child safety seat; and
determine and recommend, a suitability of the child safety seat.

57. The system of claim 56, wherein the sensor module further comprises one or more of a seat belt sensor, a position sensor, a pressure sensor, a weight sensor, a motion sensor, an infrared sensor, a lidar sensor, an optical sensor, a moisture sensor, a temperature sensor, a laser sensor, an ultrasonic sensor, a level sensor, and a time-of-flight sensor.

58. The system of claim 56, wherein the radar sensor is a millimeter-wave (mmWave) radar sensor; and wherein the image sensor is a camera comprising a computer vision module.

59. The system of claim 56, wherein the first characteristic of the child safety seat comprises one or more of a make, a model, a shape, and a size of the child safety seat.

60. The system of claim 56, wherein the specification information of the child safety seat comprises one or more of type of the child safety seat, a positioning of the child safety seat, an orientation of the child safety seat, a feature of the child safety seat, a height limit, a weight limit, an age limit; and wherein the orientation of the child safety seat comprises one of a rear facing child safety seat, a front facing child safety seat, a convertible child safety seat, and an all-in-one child safety seat.

61. The system of claim 56, wherein the first characteristic is utilized to retrieve the specification information from data stored in a database stored in one of a local memory and a cloud memory.

62. The system of claim 56, wherein the system further comprises a communication module connecting the vehicle and the child safety seat; and wherein the first characteristic is received via the communication module.

63. The system of claim 56, wherein the second characteristic of the child comprises one or more of a height of the child, a weight of the child, an age of the child, a behavior of the child, and a fit of the child in the child safety seat.

64. The system of claim 56, wherein the system further comprises a display is operable to display a message, wherein the message comprises a position the child safety seat, a direction of the child safety seat, and the suitability of the child safety seat.

65. The system of claim 56, wherein determining the suitability comprises information that indicates at least one of continuing use of the child safety seat, a replacement for the child safety seat, and an upgradation of the child safety seat.

66. The system of claim 56, wherein the system further provides an alert signal upon determining a necessity for at least one of a replacement of the child safety seat and an upgradation of the child safety seat, wherein the alert signal comprises one or more of a text message, a visual cue, a sound alert, a tactile cue, and a vibration.

67. The system of claim 56, wherein the suitability of the child safety seat is determined by comparing a height of the child, a weight of the child, an age of the child, to a height limit, a weight limit, an age limit of the specification information of the child safety seat.

68. A method comprising:
sensing, at least a portion of an interior of a vehicle, via a sensor module, wherein the sensor module comprises a radar sensor and an image sensor;
acquiring, information of the interior of the vehicle, via the sensor module;
determining, using the information of the interior of the vehicle via a machine learning module, a first characteristic of a child safety seat positioned in the interior of the vehicle;
retrieving, using the first characteristic of the child safety seat positioned in the interior of the vehicle, a specification information of the child safety seat;
determining, using the information of the interior of the vehicle via the machine learning module, a second characteristic of a child positioned in the child safety seat;
comparing, the second characteristic of the child with the specification information of the child safety seat; and
determining and recommending, a suitability of the child safety seat.

69. The method of claim 68, wherein the image sensor is a camera comprising computer vision module and is operable for capturing a video in real-time enabling continuous monitoring, wherein the video is analyzed for detecting a behavior of the child, wherein the behavior comprises one or more of an attempt to unbuckle a harness of the child safety seat, a restlessness of the child seated in the child safety seat, an attempt to exit the child safety seat, and a legroom available for the child in the child safety seat.

70. The method of claim 68, wherein the machine learning module is operable for detecting and categorizing a child behavior from one or more of a height of the child, a weight of the child, a movement of the child, a posture of the child, a gesture of the child, a health parameter of the child, and a face detection of the child; and wherein the machine learning module comprises one or more of an object detection algorithm, a facial recognition algorithm, a pose estimation algorithm, a gesture recognition algorithm, a voice recognition algorithm, a gait recognition algorithm, and an activity recognition algorithm.

71. The method of claim 68, wherein the second characteristic of the child comprises a height of the child, a weight of the child, an age of the child, a behavior of the child, and a fit of the child in the child safety seat.

72. A non-transitory computer-readable medium having stored thereon instructions executable by a computer system to perform operations comprising:
sensing, at least a portion of an interior of a vehicle, via a sensor module, wherein the sensor module comprises a radar sensor and an image sensor;
acquiring, information of the interior of the vehicle, via the sensor module;
determining, using the information of the interior of the vehicle via a machine learning module, a first characteristic of a child safety seat positioned in the interior of the vehicle;
retrieving, using the first characteristic of the child safety seat positioned in the interior of the vehicle, a specification information of the child safety seat;
determining, using the information of the interior of the vehicle via the machine learning module, a second characteristic of a child positioned in the child safety seat;
comparing, the second characteristic of the child with the specification information of the child safety seat; and
determining and recommending, a suitability of the child safety seat.

73. The non-transitory computer-readable medium of claim 72, wherein the image sensor comprises a camera comprising computer vision module operable for capturing a video in real-time and enabling continuous monitoring.

74. The non-transitory computer-readable medium of claim 73, wherein the video is analyzed for detecting a behavior of the child, wherein the behavior comprises one or more of an attempt to unbuckle a harness of the child safety seat, a restlessness of the child seated in the child safety seat, an attempt to exit the child safety seat, and a legroom available for the child in the child safety seat.

75. The non-transitory computer-readable medium of claim 72, wherein the computer system is further operable to detect one or more of an expiration date of the child safety seat, a crash history of the child safety seat, and a recall notification for the child safety seat.
